(19) United States
(12) Patent Application Publication
(10) Pub. No.: US 2026/0207468 A1
(43) Pub. Date: Jul. 23, 2026

(54) PROCESS FOR TREATING KERATIN MATERIALS USING AT LEAST ONE OIL BEARING ACETOACETATE FUNCTIONS AND AT LEAST ONE CROSSLINKING AGENT

(71) Applicant: L'OREAL, PARIS (FR)

(72) Inventors: Laurent SABATIE, AULNAY-SOUS-BOIS (FR); Etienne SAVONNET, AULNAY-SOUS-BOIS (FR); Boris STEVANT, AULNAY-SOUS-BOIS (FR)

(21) Appl. No.: 19/142,421

(22) PCT Filed: Dec. 26, 2023
(86) PCT No.: PCT/EP2023/087792

§ 371 Date: Jun. 23, 2025

(30) Foreign Application Priority Data
Dec. 27, 2022 (FR) FR2214549

(51) Int. Cl. A61K 8/92; A61K 8/31; A61K 8/34; A61K 8/41; A61K 8/45; A61K 8/58; A61K 8/898; A61K 8/899; A61Q 1/02; A61Q 1/06; A61Q 1/08; A61Q 1/10

(52) U.S. Cl. CPC A61K 8/922; A61K 8/31; A61K 8/34; A61K 8/41; A61K 8/45; A61K 8/585; A61K 8/898; A61K 8/899; A61Q 1/025; A61Q 1/06; A61Q 1/08; A61Q 1/10; A61K 2800/43; A61K 2800/884

(57) ABSTRACT

Process for treating keratin materials using at least one oil bearing acetoacetate functions and at least one crosslinking agent

The present invention relates to a process for treating keratin materials, notably the skin and/or the lips and/or the eyelashes and/or the eyebrows, comprising the application to said keratin materials, notably the skin and/or the lips and/or the eyelashes and/or the eyebrows, in one or more successive steps, of at least i) one or more compounds of formula (I), and also the optical or geometrical isomers thereof and/or the solvates thereof, such as hydrates, or a composition containing same:

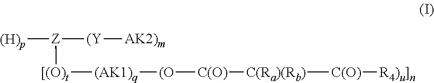

in which formula (I) Z, AK1, AK2, Y, R₄, Rₐ, R_{b}, p, m, n, t, q and u are as defined in the description, ii) at least one crosslinking agent.

### TECHNICAL FIELD

The present invention relates more particularly to the cosmetic field, and notably to that of caring for and/or making up keratin materials, notably the skin and/or the lips and/or the eyelashes and/or the eyebrows.

The aim is thus notably to propose novel treatment processes, notably cosmetic treatment processes, comprising the application to keratin materials, notably the skin and/or the lips and/or the eyelashes and/or the eyebrows, of an oil bearing acetoacetate functions and of at least one crosslinking agent, which are most particularly advantageous in terms of their technical performance, notably in terms of the resistance of the deposits they make it possible to obtain, and the sensorial experience they give the user.

### PRIOR ART

Cosmetic products conventionally require the use of one or more film-forming polymers in order to obtain a quality deposit of these products on keratin materials, and in particular to satisfy the expectations detailed below.

Thus, in the field of making up the skin and/or the lips, it is most particularly expected that the deposit formed does not transfer when in contact with fingers or clothing. It should also have good staying power on contact with water, notably rain or during showering or even perspiration, and also with sebum, or even in contact with fatty food substances, notably food oils when this deposit is formed on the lips. Moreover, this deposit must be comfortable, or even glossy.

For this purpose, dispersions of polymer particles of nanometric size are used in makeup products, such as mascaras, eyeliners, eyeshadows or lipsticks, and in hair application products, and more particularly in their organic and notably oily phases, as film-forming agent.

However, the compositions of the prior art have unsatisfactory staying power over time.

Compositions are known, notably film-forming cosmetic compositions, comprising at least one fatty phase and at least one copolymer obtained by polymerization of ethylenic monomers, some of which bear particular groups (see, for example, WO 2022/136104). Such compositions nevertheless require the presence of a fatty phase, which may reduce the field to certain applications. Furthermore, these compositions do not necessarily meet the need for naturalness expressed by certain consumers.

It is also known practice from patent EP 1 392 222 to use a cosmetic composition for caring for and/or treating keratin materials, comprising a supramolecular polymer including a polymer backbone and at least two groups that are capable of forming at least three hydrogen bonds. However, with this composition alternative, the obtained performance with respect to staying power over time or with respect to water resistance remains insufficient.

Compositions and processes for forming deposits on keratin substrates having good staying power over time, using compounds bearing acetoacetate functions and functionalized active agents for being covalently bonded to the keratin materials are also known from WO 2014/099108.

Moreover, WO 06/042169 describes tissue adhesives, in medical and veterinary applications, formed by the reaction of polyhydroxy compounds derived with acetoacetate groups and/or polyamine compounds derived with acetoacetamide groups with an amino-functional crosslinking compound.

The synthesis of acetoacetate diester triglycerides is also described in WO 2016/164870. However, in general, the treatments and processes described above do not make it possible to obtain deposits, on keratin materials, in particular on the skin and/or the lips, which satisfy all the abovementioned requirements, namely very good resistance to water and/or to fatty substances, notably for the lips, which are moreover comfortable to wear for the users, which make it possible to adjust the gloss of the deposits or which do not produce deposits that are visually unsightly, transparent and/or invisible if not coloured.

Moreover, there is considerable consumer demand for compositions with a reduced environmental impact.

### DISCLOSURE OF THE INVENTION

There is thus still a need for a treatment process, notably a cosmetic treatment process, intended for application to the keratin materials, notably the skin and/or the lips and/or the eyelashes and/or the eyebrows, which makes it possible to obtain a deposit that is film-forming, non-tacky, which transfers little, if at all, and which is glossy, comfortable and persistent.

There is also still a need for a treatment process, notably a cosmetic treatment process, which allows deposits to be obtained with a very good staying power over time, that are resistant to water and fatty substances, in particular sebum.

There is also still a need for a treatment process, notably a cosmetic treatment process, which affords deposits that have shiny and/or invisibility qualities.

The present invention is specifically directed toward meeting all or some of these needs.

### SUMMARY OF THE INVENTION

These problems are solved by performing a process for treating, notably a cosmetic process for treating, keratin materials, notably the skin and/or the lips and/or the eyelashes and/or the eyebrows, comprising the application to said keratin materials, notably the skin and/or the lips and/or the eyelashes and/or the eyebrows, in one or more successive steps, of at least:
i) one or more compounds of formula (I), and also the optical or geometrical isomers thereof, and/or solvates thereof, such as hydrates, or a composition containing same:

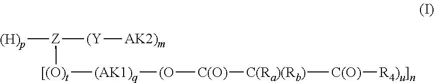

in which formula (I):
Z represents a linear or branched, saturated or unsaturated, conjugated or non-conjugated, acyclic or cyclic, aromatic or non-aromatic multivalent C₂ to C₄₀, in particular C₃ to C₃₆, hydrocarbon-based radical, Z possibly being interrupted with one or more groups or heteroatoms chosen from —O—, —N(Rₐ)—, —S—, —C(O)—, (hetero)cycles, and combinations thereof;
AK1 represents a divalent saturated or unsaturated, linear or branched C₁-C₁₂, preferably C₁-C₆, more preferentially saturated C₁-C₆, hydrocarbon-based chain;
AK2 represents a linear or branched, saturated or unsaturated, C₁-C₄₀ monovalent hydrocarbon-based chain, optionally substituted with one or more identical or different radicals chosen from —OR, —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), and combinations thereof, in particular —O—C(O)—, —C(O)—O— and/or -(hetero)cycle-O-(hetero)cycle-;
AK3 represents a divalent linear or branched, saturated or unsaturated C₁-C₁₂, preferably C₁-C₆, more preferentially saturated C₁-C₆, even more preferentially C₁-C₄, hydrocarbon-based chain;
R represents a hydrogen atom, a linear or branched, saturated or unsaturated C₁-C₆, preferably C₁-C₄, alkyl radical, in particular a methyl radical, or a radical —C(O)—C(Rₐ)(R_{b})—C(O)—R₄;
X represents —O—, —S—, —O—C(O)— or —C(O)—O—;
Y represents —O—C(O)— or —C(O)—O—;
R₄ represents a linear or branched, saturated or unsaturated C₁-C₆ monovalent hydrocarbon-based radical, preferably a (C₁-C₄)alkyl group, in particular methyl or tert-butyl, more preferentially methyl;
Rₐ and R_{b}, which may be identical or different, represent a hydrogen atom or a (C₁-C₄)alkyl group, preferably a hydrogen atom;
p denotes an integer equal to 0 or 1;
m denotes an integer equal to 0, 1, 2, 3 or 4; it being understood that when m is greater than 1, then the radicals —Y-AK2 are identical or different;
n denotes an integer equal to 0, 1, 2, 3 or 4; it being understood that when n is greater than 1, then the radicals —(O)ₜ-(AK1)_{q}—(O—C(O)—C(Rₐ)(R_{b})—C(O)—R₄)ᵤ are identical or different;
t denotes an integer equal to 0 or 1;
q denotes an integer equal to 0 or 1;
u denotes an integer equal to 1, 2 or 3;
v denotes an integer equal to 1, 2 or 3;
   it being understood that if q is equal to 0 then t is equal to 0, that (m+n) is greater than or equal to 1, and that the compounds of formula (I) contain at least two groups —C(O)—C(Rₐ)(R_{b})—C(O)—R₄; and
ii) at least one crosslinking agent.

According to a preferred embodiment, the treatment process according to the invention also comprises the application of at least iii) a cosmetic active agent, to the keratin materials, notably the skin and/or the lips and/or the eyelashes and/or the eyebrows.

According to a preferred embodiment, the process according to the invention comprises the application i) of at least one compound as defined previously, the optical or geometrical isomers thereof, and/or the solvates thereof, such as hydrates, or of a composition containing same, the application ii) of at least one crosslinking agent and the application iii) of at least one cosmetic active agent, to the keratin materials, notably the skin and/or the lips and/or the eyelashes and/or the eyebrows.

When it is present, the cosmetic active agent iii) may be applied at the same time as i) and/or as ii).

According to a particular embodiment, the keratin materials, notably the skin and/or the lips and/or the eyelashes and/or the eyebrows, may be damp or wet before application of i) and/or of ii), and possibly iii). By way of example, steps i), ii) and optionally iii), may be performed on damp skin.

According to one embodiment, the process comprises the application of i), ii) and optionally iii) to damp or wet keratin materials, notably to damp or wet skin and/or lips and/or eyelashes and/or eyebrows.

According to another embodiment, the process comprises the application of i), ii) and optionally iii) to dry keratin materials, notably dry skin and/or lips and/or eyelashes and/or eyebrows.

The inventors thus found, surprisingly, that the application to keratin materials, notably the skin and/or the lips and/or the eyelashes and/or the eyebrows, of the ingredients i), ii), and optionally iii), makes it possible to obtain deposits on keratin materials, notably the skin, which have good resistance to external attacking factors, for instance water, oils, notably food oils, sweat and/or sebum, and which are thus endowed with very good staying power over time.

Advantageously, the deposits obtained via the processes according to the invention are also very comfortable. They are not tacky and do not transfer.

Applying ingredients i), ii), and possibly iii) to keratin materials, notably to the skin and/or the lips and/or the eyelashes and/or the eyebrows also affords advantageously shiny deposits.

### Definitions

For the purposes of the present invention, throughout the description, including the claims, and unless otherwise indicated:
The term “cosmetic active agent” means an organic or organosilicon compound or a mineral compound which can be incorporated into a cosmetic composition to give an effect on keratin materials, whether this effect is immediate or provided by repeated applications. As examples of cosmetic active agents, mention may be made of coloured or uncoloured, fluorescent or non-fluorescent compounds such as optical brighteners, or UVA and/or UVB screening agents, care active agents such as anti-ageing active agents or active agents intended for providing a benefit to keratin materials, notably to the skin, such as active agents having an action on the barrier function, deodorant active agents, antiperspirant active agents, desquamating active agents, antioxidant active agents, moisturizing active agents, sebum-regulating active agents, active agents intended for combating the effects of pollution, antimicrobial or bactericidal active agents, fragrances and dyestuffs such as direct dyes or pigments, preferably pigments.

Preferentially, the cosmetic active agents are chosen from a) dyestuffs chosen from pigments, direct dyes, and mixtures thereof, b) active agents for caring for keratin materials, preferably the skin, c) UV-screening agents, and d) mixtures thereof.
For the purposes of the present invention, the term “fatty substance” means an organic compound that is insoluble in water at ordinary temperature (25° C.) and at atmospheric pressure (760 mmHg) (solubility of less than 5%, preferably less than 1% and even more preferentially less than 0.1%); in addition, the fatty substances are soluble in organic solvents under the same temperature and pressure conditions, for instance in halogenated solvents such as chloroform or dichloromethane, lower alcohols such as ethanol or aromatic solvents such as benzene or toluene.
The term “(hetero)aryl” means aryl or heteroaryl groups.
The term “(hetero)cycloalkyl” means cycloalkyl or heterocycloalkyl groups.
The “aryl” or “heteroaryl” radicals or the aryl or heteroaryl part of a radical may be substituted with at least one substituent borne by a carbon atom, chosen from:
   a C₁-C₆ and preferably C₁-C₄ (poly)(hydroxy)alkyl radical;
   a halogen atom such as chlorine, fluorine or bromine;
   a hydroxyl group;
   a C₁-C₂ alkoxy radical; a C₂-C₄ (poly)hydroxyalkoxy radical;
   an amino radical;
   an amino radical substituted with one or two identical or different C₁-C₆, and preferably C₁-C₄, alkyl radicals;
   an acylamino radical (—N(R)—C(O)—R′) in which the radical R is a hydrogen atom;
   a C₁-C₄ alkyl radical and the radical R′ is a C₁-C₄ alkyl radical; a carbamoyl radical ((R)₂N—C(O)—) in which the radicals R, which may be identical or different, represent a hydrogen atom or a C₁-C₄ alkyl radical;
   an alkylsulfonylamino radical (R′—S(O)₂—N(R)—) in which the radical R represents a hydrogen atom or a C₁-C₄ alkyl radical and the radical R′ represents a C₁-C₄ alkyl radical, or a phenyl radical;
   an aminosulfonyl radical ((R)₂N—S(O)₂—) in which the radicals R, which may be identical or different, represent a hydrogen atom or a C₁-C₄ alkyl radical;
   a carboxylic radical in acid form or salified (preferably with an alkali metal or a substituted or unsubstituted ammonium) form;
   a cyano group (CN);
   a polyhalo(C₁-C₄)alkyl group, preferentially trifluoromethyl (CF₃).
The cyclic or heterocyclic part of a non-aromatic radical may be substituted with at least one substituent borne by a carbon atom, chosen from the following groups:
   hydroxyl,
   C₁-C₄ alkoxy, C₂-C₄ (poly)hydroxyalkoxy;
   alkylcarbonylamino ((R—C(O)—NR′—), in which the radical R′ is a hydrogen atom or a C₁-C₄ alkyl radical and the radical R is a C₁-C₂ alkyl radical, amino substituted with one or two identical or different C₁-C₄ alkyl groups;
   alkylcarbonyloxy((R—C(O)—O—), in which the radical R is a C₁-C₄ alkyl radical, amino substituted with one or two identical or different C₁-C₄ alkyl groups;
   alkoxycarbonyl ((R—O—C(O)—) in which the radical R is a C₁-C₄ alkyl radical, amino substituted with one or two identical or different C₁-C₄ alkyl groups;
A cyclic or heterocyclic radical, or a non-aromatic part of an aryl or heteroaryl radical, may also be substituted with one or more oxo groups.
A hydrocarbon-based chain is unsaturated when it includes one or more double bonds and/or one or more triple bonds.
An “aryl” radical represents a monocyclic or fused or non-fused polycyclic hydrocarbon-based group comprising from 6 to 14 carbon atoms, and at least one ring of which is aromatic; preferentially, the aryl radical is a phenyl, biphenyl, naphthyl, indenyl, anthracenyl or tetrahydronaphthyl.
A “heteroaryl” radical represents a monocyclic or fused or non-fused polycyclic, 5- to 14-membered group, comprising from 1 to 6 heteroatoms chosen from nitrogen, oxygen, sulfur and selenium atoms, and at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridyl, tetrazolyl, dihydrothiazolyl, imidazopyridyl, imidazolyl, indolyl, isoquinolyl, naphthoimidazolyl, naphthooxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridyl, thiazoylimidazolyl, thiopyrylyl, triazolyl and xanthylyl.
A “cyclic” or “cycloalkyl” radical is a monocyclic or fused or non-fused polycyclic, non-aromatic cyclic hydrocarbon-based radical containing from 5 to 14 carbon atoms, which may include one or more unsaturations; the cycloalkyl is preferably a cyclohexyl group.
A “heterocyclic” or “heterocycloalkyl” radical is a monocyclic or fused or non-fused polycyclic 3- to 9-membered non-aromatic cyclic radical, including from 1 to 4 heteroatoms chosen from nitrogen, oxygen, sulfur and selenium atoms; preferably, the heterocycloalkyl is chosen from epoxide, piperazinyl, piperidyl, morpholinyl and dithiolane.
An “alkyl” radical is a linear or branched, in particular C₁-C₆ and preferably C₁-C₄ saturated hydrocarbon-based radical.
An “alkoxy” radical is an alkyl-oxy radical for which the alkyl radical is a linear or branched C₁-C₆, and preferentially C₁-C₄, hydrocarbon-based radical.
A “(poly)(hydroxy)alkyl” radical denotes a C₁-C₆ and preferably C₁-C₄ alkyl radical, optionally substituted with one or more hydroxyl radicals, preferably substituted with from 1 to 4 hydroxyl groups, more particularly between 1 and 3.
A “sugar” radical is a monosaccharide or disaccharide radical. Sugar radicals that may be mentioned include: sucrose, glucose, galactose, ribose, fucose, maltose, fructose, mannose, arabinose, xylose, lactose.
The term “monosaccharide” refers to a mono-oside sugar comprising at least 5 carbon atoms of formula Cₓ(H₂O)ₓ with x an integer greater than or equal to 5, preferably x is greater than or equal to 6, in particular x is between 5 and 7 inclusive, preferably x is equal to 6; they may be of D or L configuration, and of alpha or beta anomer, and also the salts thereof and the solvates thereof such as hydrates.
The term “disaccharide” refers to a di-oside sugar which is a compound constituted of two saccharides bonded together via O-oside bonds, said compounds being constituted of two monosaccharide units (also known as mono-osides) as defined previously, said monosaccharide units comprising at least 5 carbon atoms, preferably 6; in particular, the mono-oside units are linked together via a 1,4 or 1,6 bond as α (alpha) or β (beta) anomer, it being possible for each oside unit to be of L or D configuration, and also the salts thereof and the solvates thereof such as the hydrates of said monosaccharides; more particularly, they are polymers formed from two saccharides (or monosaccharides) having the general formula: —[Cₓ(H₂O)_{y})]₂— or —[(CH₂O)ₓ]₂—, with x being an integer greater than or equal to 5, preferably x is greater than or equal to 6, in particular x is between 5 and 7 inclusive, preferably x is equal to 6, and y is an integer representing x−1.
The term “polysaccharide” refers to a polysaccharide sugar which is a polymer constituted of several saccharides bonded together via O-oside bonds, said polymers being constituted of monosaccharide units (also known as mono-osides) as defined previously, said monosaccharide units comprising at least 5 carbon atoms, preferably 6; in particular, the monosaccharide units are linked together via a 1,4 or 1,6 bond as α (alpha) or β (beta) anomer, it being possible for each oside unit to be of L or D configuration, and also the salts thereof and the solvates thereof such as the hydrates of said monosaccharides; more particularly, they are polymers formed from a certain number of saccharides (or monosaccharides) having the general formula: —[Cₓ(H₂O)_{y})]_{w}— or —[(CH₂O)ₓ]_{w}—, where x is an integer greater than or equal to 5, preferably x is greater than or equal to 6, in particular x is between 5 and 7 inclusive and preferably x is equal to 6, and y is an integer which represents x−1, and w is an integer greater than or equal to 2, particularly between 3 and 3000 inclusive, more particularly between 5 and 2500, preferentially between 10 and 2300, particularly between 15 and 1000 inclusive, more particularly between 20 and 500, preferentially between 25 and 200.
An “anionic counterion” is an anion or an anionic group associated with the cationic charge; more particularly, the anionic counterion is chosen from: i) halides such as chloride or bromide; ii) nitrates; iii) sulfonates, including C₁-C₆ alkylsulfonates: Alk-S(O)₂O⁻ such as methanesulfonate or mesylate and ethanesulfonate; iv) arylsulfonates: Ar—S(O)₂O⁻ such as benzenesulfonate and toluenesulfonate or tosylate; v) citrate; vi) succinate; vii) tartrate; viii) lactate; ix) alkyl sulfates: Alk-O—S(O)O⁻ such as methyl sulfate and ethyl sulfate; x) aryl sulfates: Ar—O—S(O)O⁻ such as benzene sulfate and toluene sulfate; xi)alkoxy sulfates: Alk-O—S(O)₂O⁻ such as methoxy sulfate and ethoxy sulfate; xii) aryloxy sulfates: Ar—O—S(O)₂O⁻; xiii) phosphate; xiv) acetate; xv) triflate; and xvi) borates such as tetrafluoroborate.
The “solvates” represent hydrates and also the combination with linear or branched C₁-C₄ alcohols such as ethanol, isopropanol or n-propanol.
The term “UV-A screening agent” means a chromophore derived from a compound which screens out (or absorbs) UV-A ultraviolet rays at a wavelength of between 320 and 400 nm. A distinction may be made between short UV-A screening agents (which absorb rays at a wavelength of between 320 and 340 nm) and long UV-A screening agents (which absorb rays at a wavelength of between 340 and 400 nm).
The term “UV-B screening agent” means a chromophore derived from a compound which screens out (or absorbs) UV-B ultraviolet rays at a wavelength of between 280 and 320 nm.
The term “chromophore” means a radical derived from a colourless or coloured compound that is capable of absorbing UV and/or visible radiation at a wavelength λ_{abs} of between 250 and 800 nm. Preferably, the chromophore is coloured, i.e. it absorbs wavelengths in the visible range, i.e. preferably between 400 and 800 nm. Preferably, the chromophores appear coloured to the eye, particularly between 400 and 700 nm (Ullmann's Encyclopedia, 2005, Wiley-VcH, Verlag “Dyes, General Survey”, § 2.1 Basic Principle of Color);
The term “anhydrous composition” means that said composition contains an amount of less than 5% by weight of water, preferentially less than 3% by weight of water, better still less than 1% by weight of water, relative to the total weight of the composition in question; even more preferentially, the composition under consideration is free of water.
The terms “colouring agent” and “dyestuff” are equivalent.
The expression “including a” should be understood as being synonymous with “including at least one”, unless otherwise mentioned.
The expressions “between . . . and . . . ”, “comprises from . . . to . . . ”, “formed from . . . to . . . ” and “ranging from . . . to . . . ” should be understood as being inclusive of the limits, unless otherwise specified.

It is understood that the treatment processes according to the invention, and also the compositions used, are non-therapeutic.

### DETAILED DESCRIPTION

### Treatment Process

The first subject of the invention is a treatment process in one or more steps using, on keratin materials, in particular the skin and/or the lips and/or the eyelashes and/or the eyebrows, notably the skin:
i) at least one or more compounds of formula (I), as defined previously and hereinbelow, or a composition containing same;
ii) at least one crosslinking agent, in particular as defined hereinbelow; and
iii) optionally at least one cosmetic active agent, in particular as defined hereinbelow.

According to a particular embodiment of the invention, the treatment process according to the invention involves the simultaneous application of the ingredients i), ii), and optionally iii).

According to another particular embodiment of the invention, the treatment process according to the invention comprises at least two steps in which the ingredients i), ii) and optionally iii) are applied in separate and successive steps to the keratin materials, notably the skin and/or the lips and/or the eyelashes and/or the eyebrows, it being understood that the cosmetic active agent(s) iii) may be present together with the ingredient(s) i) and/or with the ingredient(s) ii).

According to a particular embodiment, the ingredient(s) i) are applied to the keratin materials, notably the skin and/or the lips and/or the eyelashes and/or the eyebrows, and the ingredient(s) ii) are then applied to said keratin materials, notably to the skin and/or the lips and/or the eyelashes and/or the eyebrows, it being understood that ingredient iii), when it is present, may be applied with i) and/or with ii).

According to another particular embodiment, the ingredient(s) ii) are applied to the keratin materials, notably the skin and/or the lips and/or the eyelashes and/or the eyebrows, and the ingredient(s) i) are then applied to said keratin materials, notably to the skin and/or the lips and/or the eyelashes and/or the eyebrows, it being understood that ingredient iii), when it is present, may be applied with i) and/or with ii).

According to a particular embodiment of the invention, the process for treating, notably cosmetically treating, keratin materials, notably the skin and/or the lips and/or the eyelashes and/or the eyebrows, is a cosmetic treatment process for caring for and/or making up keratin materials, notably the skin, the lips, the eyelashes and/or eyebrows.

According to an embodiment of the invention, the process for treating keratin materials, notably the skin and/or the lips and/or the eyelashes and/or the eyebrows, according to the invention using the ingredients i), ii), and optionally iii), is a skincare process.

According to an embodiment of the invention, the process for treating keratin materials, notably the skin and/or the lips and/or the eyelashes and/or the eyebrows, is a process for making up keratin materials, notably the skin, the lips, the eyelashes and/or the eyebrows.

According to this embodiment, the process comprises at least one cosmetic active agent chosen from dyestuffs, preferably pigments.

When the treatment process involves several steps including a step of using one or more compounds of formula (I), (Ia), (Ib), (Ic), (Id), (Ie), (IIc), (IId) and/or (IIe) without crosslinking agent, the compound(s) of formula (I), (Ia), (Ib), (Ic), (Id), (Ie), (IIc), (IId) and/or (IIe) may be used on their own (pure), or in a composition “C1” as described hereinbelow.

According to one embodiment of the invention, the process according to the invention is a process for treating, notably for cosmetically treating, keratin materials, notably the skin and/or the lips and/or the eyelashes and/or the eyebrows, in particular for caring for and/or making up keratin materials, notably the skin, the lips, the eyelashes and/or eyebrows, comprising the application to said keratin materials, notably the skin and/or the lips and/or the eyelashes and/or the eyebrows, of at least:
a composition, termed “C1”, comprising i) at least one compound of formula (I), or (Ia), (Ib), (Ic), (Id) and/or (Ie), as defined previously and hereinbelow, and optionally iii) at least one cosmetic active agent, in particular as defined previously and hereinbelow; preferably, composition “C1” does not comprise iii) a crosslinking agent;
a composition, termed “C2”, comprising i) at least one compound of formula (I), or (Ia), (Ib), (Ic), (Id) and/or (Ie), as defined previously and hereinbelow, ii) at least one crosslinking agent, notably as defined hereinbelow, and optionally iii) at least one cosmetic active agent, in particular as defined previously and hereinbelow; preferably, composition “C2” does not comprise iv) a cosmetic active agent;
a composition, termed “C3”, comprising i) at least one compound of formula (I), or (Ia), (Ib), (Ic), (Id) and/or (Ie), as defined previously and hereinbelow, ii) at least one crosslinking agent, in particular as defined hereinbelow, and iii) at least one cosmetic active agent, in particular as defined previously and hereinbelow;
a composition, termed “C4”, comprising ii) at least one crosslinking agent, in particular as defined hereinbelow, and optionally iii) at least one cosmetic active agent, in particular as defined previously and hereinbelow; preferably, composition “C4” does not comprise i) at least one compound of formula (I), or (Ia), (Ib), (Ic), (Id) and/or (Ie) as defined previously and hereinbelow; and/or
a composition, termed “C5”, comprising iii) at least one cosmetic active agent, in particular as defined previously and hereinbelow; preferably, composition “C5” does not comprise i) at least one compound of formula (I), or (Ia), (Ib), (Ic), (Id) and/or (Ie) as defined previously and hereinbelow, and does not comprise ii) at least one crosslinking agent; it being understood that the process uses together or separately i) at least one compound of formula (I), or (Ia), (Ib), (Ic), (Id) and/or (Ie) as defined previously and hereinbelow and ii) at least one crosslinking agent, in particular as defined hereinbelow, and that the compositions “C1”, “C2”, “C3”, “C4” and “C5” may be anhydrous, aqueous such as aqueous-alcoholic and/or comprise one or more fatty substances iv), in particular as described hereinbelow.

According to a particular embodiment, the process according to the invention is a process for treating, notably for cosmetically treating, keratin materials, notably the skin and/or the lips and/or the eyelashes and/or the eyebrows, in particular for caring for and/or making up keratin materials, notably the skin, the lips, the eyelashes and/or eyebrows, comprising the application to said keratin materials, notably the skin and/or the lips and/or the eyelashes and/or the eyebrows, of at least:
i) at least one compound of formula (I), or (Ia), (Ib), (Ic), (Id) and/or (Ie), as defined previously and hereinbelow, and optionally iii) at least one cosmetic active agent, in particular as defined previously and hereinbelow;
a composition, termed “C2”, comprising i) at least one compound of formula (I), or (Ia), (Ib), (Ic), (Id) and/or (Ie), as defined previously and hereinbelow, ii) at least one crosslinking agent, notably as defined hereinbelow, and optionally iii) at least one cosmetic active agent, in particular as defined previously and hereinbelow; preferably, composition “C2” does not comprise iv) a cosmetic active agent;
a composition, termed “C3”, comprising i) at least one compound of formula (I), or (Ia), (Ib), (Ic), (Id) and/or (Ie), as defined previously and hereinbelow, ii) at least one crosslinking agent, in particular as defined hereinbelow, and iii) at least one cosmetic active agent, in particular as defined previously and hereinbelow;
a composition, termed “C4”, comprising ii) at least one crosslinking agent, in particular as defined hereinbelow, and optionally iii) at least one cosmetic active agent, in particular as defined previously and hereinbelow; preferably, composition “C4” does not comprise i) at least one compound of formula (I), or (Ia), (Ib), (Ic), (Id) and/or (Ie) as defined previously and hereinbelow; and/or
a composition, termed “C5”, comprising iii) at least one cosmetic active agent, in particular as defined previously and hereinbelow; preferably, composition “C5” does not comprise i) at least one compound of formula (I), or (Ia), (Ib), (Ic), (Id) and/or (Ie) as defined previously and hereinbelow, and does not comprise ii) at least one crosslinking agent; it being understood that the process uses together or separately i) at least one compound of formula (I), or (Ia), (Ib), (Ic), (Id) and/or (Ie) as defined previously and hereinbelow and ii) at least one crosslinking agent, in particular as defined hereinbelow, and that the compositions “C2”, “C3”, “C4” and “C5” may be anhydrous, aqueous such as aqueous-alcoholic and/or comprise one or more fatty substances iv), in particular as described hereinbelow.

According to one aspect of the invention, the process for treating keratin materials, notably the skin and/or the lips and/or the eyelashes and/or the eyebrows, in particular for caring for and/or making up keratin materials, notably the skin, the lips, the eyelashes and/or eyebrows, uses a composition termed “C3”, comprising i) at least one compound of formula (I), or (Ia), (Ib), (Ic), (Id), (Ie), (Iic), (Iid) and/or (Iie), as defined previously and hereinbelow, ii) at least one crosslinking agent, in particular as defined previously and hereinbelow, and iii) at least one cosmetic active agent, in particular as defined hereinbelow, in particular chosen from a) colouring agents, such as pigments, direct dyes, and mixtures thereof, b) active agents for caring for keratin materials, preferably the skin, c) UV-screening agents, and d) mixtures thereof, and notably at least one colouring agent, more particularly at least one pigment.

According to another aspect, the process for treating keratin materials, notably the skin and/or the lips and/or the eyelashes and/or the eyebrows according to the invention, in particular for caring for and/or making up keratin materials, notably the skin, the lips, the eyelashes and/or eyebrows, uses a composition termed “C1” and a composition termed “C4”, said composition “C1” comprising i) at least one compound of formula (I), or (Ia), (Ib), (Ic), (Id), (Ie), (Iic), (Iid) and/or (Iie), as defined previously and hereinbelow, and optionally iii) at least one cosmetic active agent, in particular as defined previously and hereinbelow, and said composition “C4” comprising ii) at least one crosslinking agent, in particular as defined hereinbelow, and optionally iii) at least one cosmetic active agent, in particular as defined previously and hereinbelow.

According to another aspect, the process for treating keratin materials, notably the skin and/or the lips and/or the eyelashes and/or the eyebrows, according to the invention, in particular for caring for and/or making up keratin materials, notably the skin, the lips, the eyelashes and/or eyebrows, uses i) at least one compound of formula (I), or (Ia), (Ib), (Ic), (Id), (Ie), (Iic), (Iid) and/or (Iie), as defined previously and hereinbelow, and a composition termed “C4”, said composition “C4” comprising ii) at least one crosslinking agent, in particular as defined hereinbelow, and optionally iii) at least one cosmetic active agent, in particular as defined previously and hereinbelow.

According to another aspect, the process for treating keratin materials, notably the skin and/or the lips and/or the eyelashes and/or the eyebrows, according to the invention, in particular for caring for and/or making up keratin materials, notably the skin, the lips, the eyelashes and/or eyebrows, uses a composition termed “C1”, or i) at least one compound of formula (I) or (Ia), (Ib), (Ic), (Id), (Ie), (Iic), (Iid) and/or (Iie) as defined previously and hereinbelow, and a composition termed “C4” and a composition termed “C5”, said composition “C1” comprising i) at least one compound of formula (I), or (Ia), (Ib), (Ic), (Id), (Ie), (Iic), (Iid) and/or (Iie), as defined previously and hereinbelow, and optionally iii) at least one cosmetic active agent, in particular as defined previously and hereinbelow, said composition “C4” comprising ii) at least one crosslinking agent, in particular as defined hereinbelow, and optionally iii) at least one cosmetic active agent, in particular as defined previously and hereinbelow, and said composition “C5” comprising iii) at least one cosmetic active agent, in particular as defined previously and hereinbelow.

According to another aspect, the process for treating keratin materials, notably the skin and/or the lips and/or the eyelashes and/or the eyebrows, according to the invention, in particular for caring for and/or making up keratin materials, notably the skin, the lips, the eyelashes and/or eyebrows, uses a composition termed “C2” and a composition termed “C5”, said composition “C2” comprising i) at least one compound of formula (I), or (Ia), (Ib), (Ic), (Id), (Ie), (Iic), (Iid) and/or (Iie), as defined previously and hereinbelow, ii) at least one crosslinking agent, in particular as defined hereinbelow, and optionally iii) at least one cosmetic active agent, in particular as defined previously and hereinbelow, and said composition “C5” comprising iii) at least one cosmetic active agent, in particular as defined previously and hereinbelow.

According to a variant of the process of the invention, the ingredient(s) i) and optionally iii) are applied together, i.e. simultaneously, to the keratin materials, notably the skin and/or the lips and/or the eyelashes and/or the eyebrows, during a first step, and then, during a subsequent step, the ingredient(s) ii), and optionally iii), are applied to said keratin materials, notably the skin and/or the lips and/or the eyelashes and/or the eyebrows.

According to a particular embodiment, the process according to the invention comprises two successive steps in which two different compositions, preferably composition “C1” and then composition “C4”, are applied to the keratin materials, notably the skin and/or the lips and/or the eyelashes and/or the eyebrows.

According to a particular embodiment of the process, during the first step a composition “C1” or i) at least one compound of formula (I), or (Ia), (Ib), (Ic), (Id), (Ie), (Iic), (Iid) and/or (Iie) as defined previously and hereinbelow, is applied to keratin materials, notably the skin and/or the lips and/or the eyelashes and/or the eyebrows, and a composition “C4” is then applied to said keratin materials, notably the skin and/or the lips and/or the eyelashes and/or the eyebrows.

According to yet another variant of the process of the invention, ingredient(s) ii) and optionally iii) are applied to the keratin materials, notably the skin and/or the lips and/or the eyelashes and/or the eyebrows, and then ingredient(s) i), and optionally iii) at least one cosmetic active agent, in particular as defined previously, are applied to said keratin materials, notably the skin and/or the lips and/or the eyelashes and/or the eyebrows. In particular, composition “C4” is applied to the keratin materials, notably the skin and/or the lips and/or the eyelashes and/or the eyebrows, and composition “C1” or i) at least one compound of formula (I), or (Ia), (Ib), (Ic), (Id), (Ie), (Iic), (Iid) and/or (Iie) as defined previously and hereinbelow is then applied.

According to another variant of the process according to the invention, i) at least one compound of formula (I), or (Ia), (Ib), (Ic), (Id), (Ie), (Iic), (Iid) and/or (Iie) as defined previously and hereinbelow or a composition “C1” containing i) and optionally iii) is applied, and sequentially a composition “C4” containing ii), and optionally iii), preferably i) or composition “C1” being applied before composition “C4”.

Compositions “C1”, “C2”, “C3”, “C4” and/or “C5” of the process according to the invention, which comprise at least one fatty substance iv), notably at least one oil, may be in the form of a direct or inverse emulsion.

Compositions “C1”, “C2”, “C3”, “C4” and/or “C5” of the process according to the invention may thus be applied directly as such to the keratin materials, notably the skin and/or the lips and/or the eyelashes and/or the eyebrows, or may even be formed directly on the surface of the keratin materials, notably the skin and/or the lips and/or the eyelashes and/or the eyebrows.

According to the invention, three application methods known as the “one-gesture application mode”, the “two-gesture application mode” and the “three-gesture application mode” are thus distinguished.

According to one embodiment of the process of the invention, the process is performed in one gesture by applying composition “C2” or “C3”, as defined previously, to the keratin materials, notably the skin and/or the lips and/or the eyelashes and/or the eyebrows.

Thus, according to one embodiment, the treatment process according to the invention comprises a single step of applying composition “C2” or composition “C3” to the keratin materials, notably the skin and/or the lips and/or the eyelashes and/or the eyebrows.

The term “one-gesture application mode” means the direct application to the target keratin materials, notably the target skin and/or lips and/or eyelashes and/or eyebrows, of a single composition in accordance with the invention, namely composition “C2” or composition “C3”.

After application of composition “C2” or of composition “C3”, a persistent, non-tacky deposit is advantageously obtained. The deposit obtained is also resistant to food oils, water, sebum and/or friction.

According to another embodiment of the process of the invention, the process is performed in two gestures.

The term “two-gesture application mode” means the successive application, to keratin materials, notably the skin and/or the lips and/or the eyelashes and/or the eyebrows, of two different compositions, for example “C1” and “C4”, or “C2” and “C4”, or “C3” and “C4”, preferably “C1” and then “C4”, “C2” and then “C4”, or “C3” and then “C4”.

The term “two-gesture application mode” also means the sequential application, to keratin materials, notably the skin and/or the lips and/or the eyelashes and/or the eyebrows, of i) at least one compound of formula (I), or (Ia), (Ib), (Ic), (Id), (Ie), (Iic), (Iid) and/or (Iie) as defined previously and hereinbelow, and of a composition “C4”, preferably i) and then “C4”. According to another embodiment, the process of the invention is performed in three gestures.

The term “three-gesture application mode” means the sequential application of three different compositions chosen from “C1”, “C2”, “C4” and “C5”. According to this application mode, for example, according to one embodiment, successive application is performed on the keratin materials, notably the skin and/or the lips and/or the eyelashes and/or the eyebrows, α) of a composition “C1” or “C2”, then β) of a composition “C4”, then γ) of a composition “C5”, preferably “C1” then “C4” or, respectively, “C5”.

The term “three-gesture application mode” also means the sequential application of i) at least one compound of formula (I), or (Ia), (Ib), (Ic), (Id), (Ie), (Iic), (Iid) and/or (Iie) as defined previously and hereinbelow, of a composition “C4” and of a composition “C5”, i) preferably being applied before “C4”.

According to another embodiment, the sequential application is performed on the keratin materials, notably the skin and/or the lips and/or the eyelashes and/or the eyebrows, α) of a composition, for example “C4”, and β) of a composition “C1” or i) of at least one compound of formula (I), or (Ia), (Ib), (Ic), (Id), (Ie), (Iic), (Iid) and/or (Iie) as defined previously and hereinbelow, or of a composition “C2”, and also γ) of a composition “C5”, preferably composition “C4” is applied before composition “C1” or i) or “C2” or “C5”.

In the two- or three-gesture application modes, the composition applied first, for example “C1” or “C2”, preferably “C1”, is conventionally referred to as the “base coat”, and the composition(s) superposed thereon, for example “C4”, are generally referred to as the “top coat”.

In the two- or three-gesture application modes using i) at least one compound of formula (I), or (Ia), (Ib), (Ic), (Id), (Ie), (Iic), (Iid) and/or (Iie) as defined previously and hereinbelow, and where this or these compounds i) are applied first, this application of i) is also conventionally referred to as the “base coat”, and the composition(s) superposed thereon, for example “C4”, are generally referred to as the “top coat”.

After application of the various compositions “C1” or i) and “C2” to “C5”, a persistent, non-tacky deposit is advantageously obtained. The deposit obtained is also resistant to food oils and water.

According to a particular embodiment, the compositions “C1” or i) and “C2” to “C5” are applied to dry keratin materials, notably dry skin and/or lips and/or eyelashes and/or eyebrows.

According to a particular embodiment, the compositions “C1” or i) and “C2” to “C5” are applied to damp or wet keratin materials, notably damp or wet skin and/or lips and/or eyelashes and/or eyebrows, i.e. to keratin materials containing water on the surface, notably the skin and/or the lips and/or the eyelashes and/or the eyebrows containing water on the surface.

According to one aspect, the present invention relates to a process, notably a cosmetic process, for treating keratin materials, notably the skin and/or the lips and/or the eyelashes and/or the eyebrows, in particular for caring for and/or making up keratin materials, notably the skin, the lips, the eyelashes and/or eyebrows, comprising a step of applying to said keratin materials, notably to said skin or said lips or said eyelashes or said eyebrows, a composition “C3”, notably containing at least one dyestuff, in particular as defined previously, and more particularly at least one pigment.

According to another of its aspects, the present invention relates to a cosmetic treatment process for caring for and/or making up keratin materials, notably the skin, the lips, the eyelashes and/or eyebrows, comprising the successive application of at least:
i) at least one compound of formula (I), or (Ia), (Ib), (Ic), (Id) and/or (Ie), as defined previously and hereinbelow, or of a composition termed “C1” comprising same and optionally comprising iii) at least one cosmetic active agent, in particular as defined previously and hereinbelow; and then
a composition, termed “C4”, comprising ii) at least one crosslinking agent, in particular as defined hereinbelow, and optionally iii) at least one cosmetic active agent, in particular as defined previously and hereinbelow;
   at least one of the compositions “C1” and/or “C4” containing at least one dyestuff, in particular as defined previously and hereinbelow, preferably at least one pigment, and more preferentially composition “C1” comprises at least one pigment.

According to another of its aspects, the present invention relates to a cosmetic treatment process for keratin materials, notably for caring for and/or making up keratin materials, notably the skin, the lips, the eyelashes and/or eyebrows, comprising the sequential application of at least:
i) at least one compound of formula (I), or (Ia), (Ib), (Ic), (Id), (Ie), (Iic), (Iid) and/or (Iie), as defined previously and hereinbelow, or a composition “C1” as defined previously; and
a composition “C4” as defined previously; and
a composition “C5” as defined previously; and
   it being understood that compositions “C1” and/or “C4” and/or “C5” contain at least one dyestuff in particular as defined hereinbelow, preferably at least one pigment. Preferably, composition “C5” comprises at least one pigment.
   Compounds i): Oils Functionalized with Acetoacetate Functions

As indicated above, the treatment process according to the invention involves applying to keratin materials, notably the skin and/or the lips and/or the eyelashes and/or the eyebrows, at least one or more compounds of formula (I), or a composition containing same.

Thus, the treatment process according to the invention comprises the application to keratin materials, notably the skin and/or the lips and/or the eyelashes and/or the eyebrows, of at least one or more compounds of formula (I) and also the optical and geometrical isomers thereof, and/or the solvates thereof, such as the hydrates, or a composition containing same:

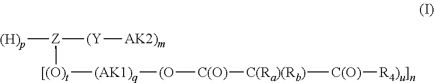

in which formula (I):
Z represents a linear or branched, saturated or unsaturated, conjugated or non-conjugated, acyclic or cyclic, aromatic or non-aromatic multivalent C₂ to C₄₀, in particular C₃ to C₃₆, hydrocarbon-based radical, Z possibly being interrupted with one or more groups or heteroatoms chosen from —O—, —N(Rₐ)—, —S—, —C(O)—, (hetero)cycle(s), and/or combinations thereof;
AK1 represents a divalent saturated or unsaturated, linear or branched C₁-C₁₂, preferably C₁-C₆, more preferentially saturated C₁-C₆, hydrocarbon-based chain;
AK2 represents a linear or branched, saturated or unsaturated, C₁-C₄₀ monovalent hydrocarbon-based chain, optionally substituted with one or more identical or different radicals chosen from —OR, —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), and combinations thereof, in particular O—(CO)—, —(CO)—O— or -(hetero)cycle-O-(hetero)cycle-;
AK3 represents a divalent linear or branched, saturated or unsaturated C₁-C₁₂, preferably C₁-C₆, more preferentially saturated C₁-C₆, even more preferentially C₁-C₄, hydrocarbon-based chain;
R represents a hydrogen atom, a linear or branched, saturated or unsaturated C₁-C₆, preferably C₁-C₄, alkyl radical, in particular a methyl radical, or a radical —C(O)—C(Rₐ)(R_{b})—C(O)—R₄;
X represents —O—, —S—, —O—C(O)— or —C(O)—O—;
Y represents —O—C(O)— or —C(O)—O—;
R₄ represents a linear or branched, saturated or unsaturated C₁-C₆ monovalent hydrocarbon-based radical, preferably a (C₁-C₄)alkyl group, in particular methyl or tert-butyl, more preferentially methyl;
Rₐ and R_{b}, which may be identical or different, represent a hydrogen atom or a (C₁-C₄)alkyl group, preferably a hydrogen atom;
p denotes an integer equal to 0 or 1;
m denotes an integer equal to 0, 1, 2, 3 or 4; it being understood that when m is greater than 1, then the radicals —Y-AK2 are identical or different;
n denotes an integer equal to 0, 1, 2, 3 or 4; it being understood that when n is greater than 1, then the radicals —(O)ₜ-(AK1)_{q}—(O—C(O)—C(Rₐ)(R_{b})—C(O)—R₄)ᵤ are identical or different;
t denotes an integer equal to 0 or 1;
q denotes an integer equal to 0 or 1;
u denotes an integer equal to 1, 2 or 3;
v denotes an integer equal to 1, 2 or 3;
   it being understood that if q is equal to 0 then t is equal to 0, that (m+n) is greater than or equal to 1, and that the compounds of formula (I) contain at least two groups —C(O)—C(Rₐ)(R_{b})—C(O)—R₄.

According to a preferred embodiment, the compound(s) of formula (I), and also the optical or geometrical isomers thereof and/or the solvates thereof, such as hydrates, are such that Z denotes a radical chosen from:
a) divalent radicals, preferably C₂-C₃₀ radicals, optionally interrupted with one or more heteroatoms or groups chosen from —O—, —N(Rₐ)—, —S—, —C(O)— and/or (hetero)cycle(s), in particular radicals —CH₂—(CH₂)_{f}, in which f denotes an integer between 1 and 10, preferably between 1 and 5, or such as the radicals —(CH₂)_{g}-(heterocycle)-O—(CH₂)ₕ-(heterocycle)-(CH₂)ⱼ—, in which h denotes 0, 1 or 2, preferably 0 or 1, and g and j, independently of each other, denote an integer between 1 and 3, preferably 1;
b) trivalent radicals, preferably C₃-C₆, in particular —CH₂—CH—CH₂—; and
c) tetravalent radicals, preferably C₃-C₁₂, in particular —CH₂—C—CH₂—CH₂—CH₂— or —CH₂—C—CH₂— or the radicals —(CH₂)ₐ—CH—CH—(CH₂)_{b}—, in which a and b independently denote an integer equal to 1, 2, 3, 4, 5, 6, 7, 8, 9 or 10; preferably, a is greater than b.

According to a preferred embodiment, a (hetero)cycle denotes a heterocyclic divalent radical (i.e. a heterocycle), preferably a heterocycloalkyl radical, more particularly a divalent 3-, 5- or 6-membered heterocycloalkyl radical such as a divalent epoxide radical, a divalent tetrahydrofuran radical or a divalent tetrahydropyran radical, said (hetero)cycle being optionally substituted with one or more radicals chosen from —OR with R as defined previously, and in particular —OH or —O—C(O)—C(Rₐ)(R_{b})—C(O)—R₄.

According to a preferred embodiment, AK2 represents a linear or branched, saturated or unsaturated, C₁₂-C₂₈ monovalent hydrocarbon-based chain, optionally substituted with one or more identical or different radicals chosen from —OR, —X-AK3-(O—C(O)—C(Rₐ)(R_{b})—C(O)—R₄)ᵥ, and/or said chain being optionally interrupted with —O—C(O)— or —C(O)—O—.

According to a particular embodiment, the compound(s) of formula (I), or (Ia), (Ib), (Ic), (Id), (Ie), (Iic), (Iid) or (Iie), as defined hereinabove and hereinbelow, are present in a composition in a content ranging from 0.1% to 99% by weight, preferably from 1% to 98% by weight, more preferentially from 5% to 96% by weight, better still from 10% to 90% by weight, even better still from 20% to 88% by weight, relative to the total weight of the composition under consideration.

According to a particular embodiment, when the treatment process according to the invention comprises several steps, the compound(s) of formula (I), or (Ia), (Ib), (Ic), (Id), (Ie), (Tic), (Iid) or (Iie), as defined hereinabove and hereinbelow, may be used pure (alone). According to a first embodiment variant, the compound(s) of formula (I), and also the optical or geometrical isomers and/or solvates thereof, such as hydrates, are such that:
Z represents the trivalent radical —CH₂—CH—CH₂—;
p is equal to 0;
n is equal to 0;
m is equal to 3;
AK2 represents a linear or branched, saturated or unsaturated, C₁₂-C₂₄ monovalent hydrocarbon-based chain, optionally substituted with one or more identical or different radicals chosen from —OR, —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, epoxides, and combinations thereof, in particular O—(CO)— or (CO)—O—;
Y, AK3, v, R, X, R₄, Rₐ and R_{b} being as defined previously for formula (I);
it being understood that the compounds contain at least two radicals —C(O)—C(Rₐ)(R_{b})—C(O)—R₄.

According to a preferred embodiment, AK2 represents a linear or branched, saturated or unsaturated, C₁₂-C₂₄ monovalent hydrocarbon-based chain, optionally substituted with one or more identical or different radicals chosen from —OR, —X-AK3-(O—C(O)—C(Rₐ)(R_{b})—C(O)—R₄)ᵥ, and/or said chain being optionally interrupted with one or more heteroatoms or groups chosen from —O—, —S—, —C(O)—, and combinations thereof, in particular —O—C(O)— or —C(O)—O—.

According to this embodiment variant, the compound(s) of formula (I) are in particular chosen from compounds (1), (2), (3), (4), (7), (8), (9) and (11) hereinbelow, and also the optical or geometrical isomers and/or solvates thereof, such as hydrates:

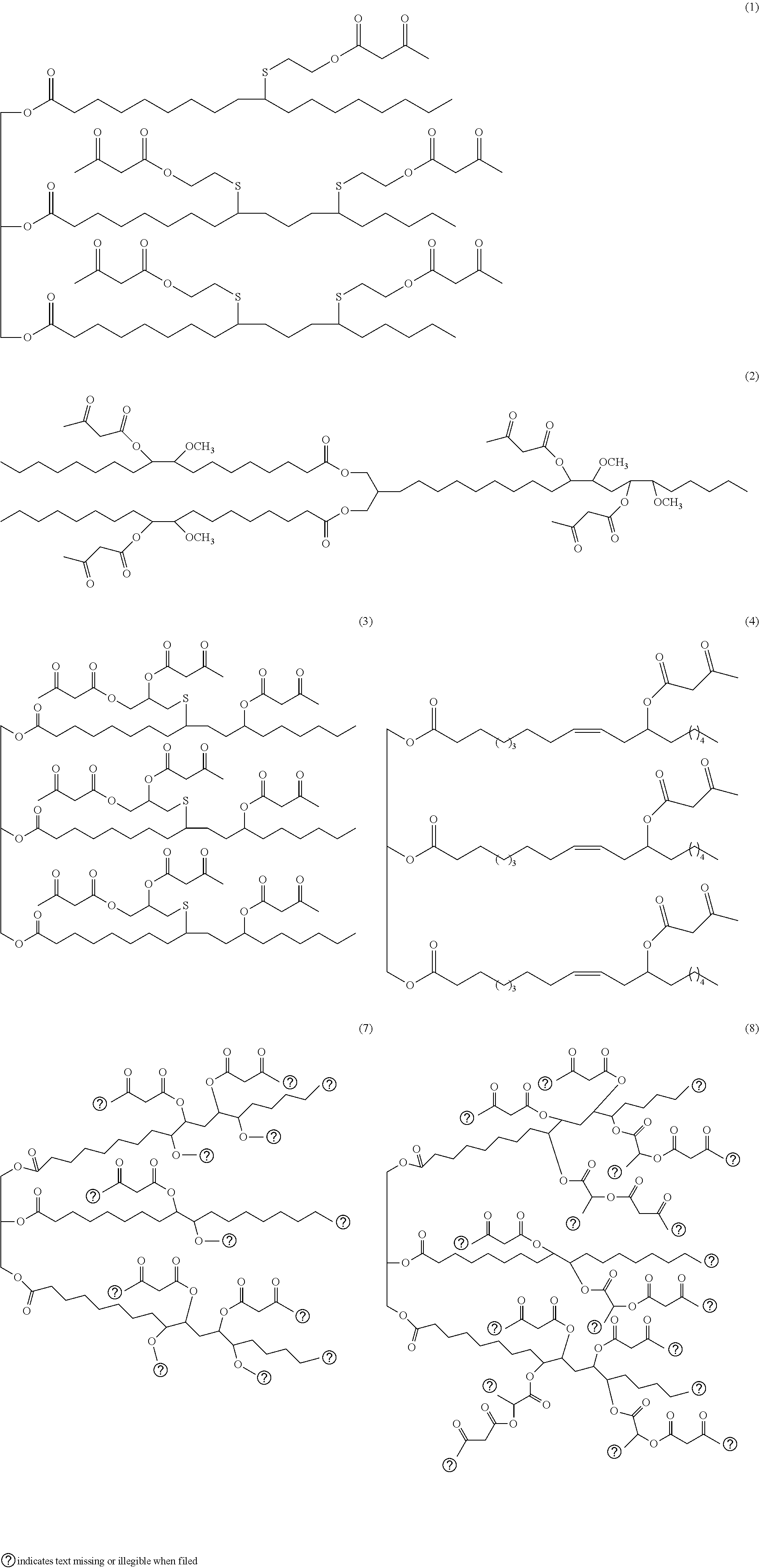

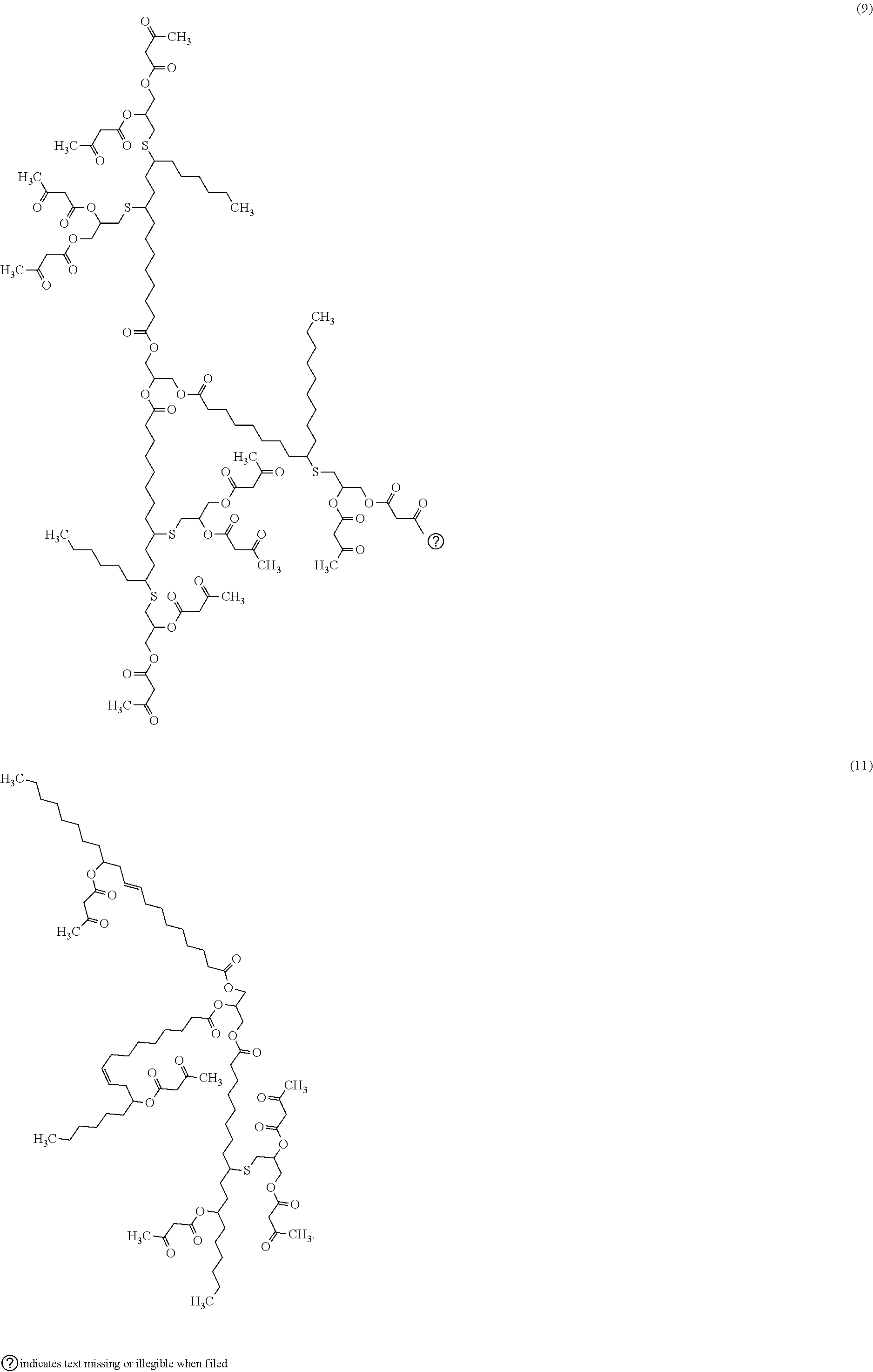 [SMILES: CCCCCCCCCC(CCCCCCCC(=O)OCC(COC(=O)CCCCCCCC(CCC(CCCCCC)SCC(COC(=O)CC(C)=O)OC(=O)CC(C)=O)SCC(COC(=O)CC(C)=O)OC(=O)CC(C)=O)OC(=O)CCCCCCCC(CCC(CCCCCC)SCC(COC(=O)CC(C)=O)OC(=O)CC(C)=O)SCC(COC(=O)CC(C)=O)OC(=O)CC(C)=O)SCC(COC(=O)CC(C)=O)OC(=O)CC(C)=O ; CCCCCCCCC(C/C=C/CCCCCCCC(=O)OCC(COC(=O)CCCCCCCC(CCC(CCCCCC)OC(=O)CC(C)=O)SCC(COC(=O)CC(C)=O)OC(=O)CC(C)=O)OC(=O)CCCCCCC/C=C\CC(CCCCCC)OC(=O)CC(C)=O)OC(=O)CC(C)=O]

According to a second embodiment variant, the compound(s) of formula (I), and also the optical or geometrical isomers and/or solvates thereof, such as hydrates, are such that:
p is equal to 0;
Z represents a linear or branched, saturated or unsaturated, preferably saturated, acyclic, multivalent C₄ to C₂₄, particularly C₆ to C₂₀, more particularly C₁₄ to C₂₀, in particular C₁₇, hydrocarbon-based radical, Z also being optionally substituted with one or more radicals —[(O)ₜ-(AK1)_{q}—(O—C(O)—C(Rₐ)(R_{b})—C(O)—R₄)ᵤ]ₙ;
n denotes an integer equal to 0, 1, 2, 3 or 4; it being understood that when n is greater than 1, then the radicals —(O)ₜ-(AK1)_{q}—(O—C(O)—C(Rₐ)(R_{b})—C(O)—R₄)ᵤ are identical or different; preferably, n is non-zero;
m is equal to 1;
AK2 represents a linear or branched, saturated or unsaturated, preferably saturated, C₃-C₈, in particular C₄ or C₆, monovalent hydrocarbon-based chain, substituted with one or more identical or different radicals chosen from —OR, —X-AK3-(O—C(O)—C(Rₐ)(R_{b})—C(O)—R₄)ᵥ, preferably substituted with one or more radicals —OR; and/or said chain being optionally interrupted with one or more heteroatoms or groups chosen from —O—, —S—, —C(O)—, (hetero)cycle(s), and combinations thereof, in particular —O—C(O)—, —C(O)—O—, -(hetero)cycle-O-(hetero)cycle-, and preferably not being interrupted;
Y, AK1, AK3, t, q, u, v, R, X, R₄, Rₐ and R_{b} being as defined previously for formula (I);
   it being understood that said compound contains at least two radicals —C(O)—C(Rₐ)(R_{b})—C(O)—R₄.

According to this embodiment variant, the compound(s) of formula (I) are in particular chosen from compounds (5) and (6) hereinbelow, and also the optical or geometrical isomers and/or solvates thereof, such as hydrates:

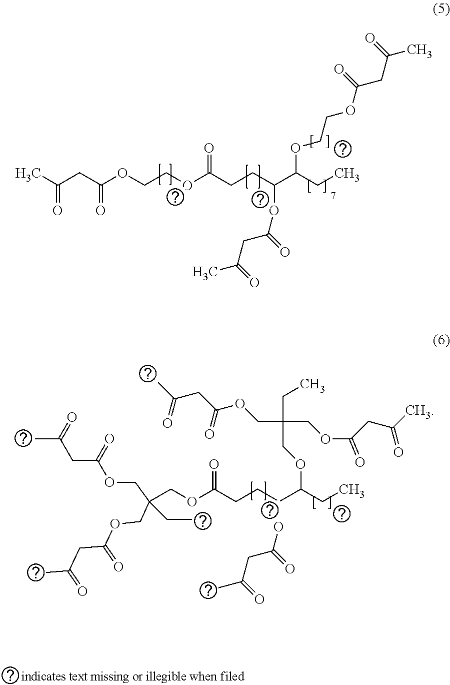

According to a third embodiment variant, the compound(s) of formula (I), and also the optical or geometrical isomers and/or solvates thereof, such as hydrates, are such that:
p is equal to 0;
n+m is equal to 4, m being non-zero;
Z represents an unsubstituted and uninterrupted, linear or branched, saturated or unsaturated, preferably saturated and acyclic, C₂ to C₂₄, particularly C₃ to C₁₀, in particular C₃, tetravalent hydrocarbon-based radical;
AK2 independently represent a linear or branched, saturated or unsaturated, preferably saturated, C₁₂-C₂₄, preferably C₁₆-C₂₀, in particular C₁₈, monovalent hydrocarbon-based chain, optionally substituted with one or more identical or different radicals chosen from —OR and —X-AK3-(O—C(O)—C(Rₐ)(R_{b})—C(O)—R₄)ᵥ, preferably substituted with one or more radicals —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), and combinations thereof, in particular —O—C(O)—, —C(O)—O—, -(hetero)cycle-O-(hetero)cycle-, and preferably not being interrupted;
AK3 represents a linear or branched, saturated or unsaturated C₁-C₁₂, preferably C₁-C₆, more preferentially saturated C₁-C₆, even more preferentially C₂-C₄, better still C₃, divalent hydrocarbon-based chain;
v is as defined previously for formula (I); preferably v is equal to 2;
X is as defined previously for formula (I); preferably, X denotes a sulfur atom;
Y, AK1, u, t, q, R, R₄, Rₐ and R_{b} being as defined previously for formula (I); it being understood that said compound contains at least two radicals —C(O)—C(Rₐ)(R_{b})—C(O)—R₄.

According to a preferred embodiment, m is equal to 3, n is equal to 1 and u is equal to 1.

According to a preferred embodiment, t is equal to 0.

According to a preferred embodiment, q is equal to 0.

According to a preferred embodiment, Y denotes —C(O)—O.

According to a preferred embodiment, none of the radicals AK2 are interrupted.

According to a preferred embodiment, the radicals AK2 are saturated.

According to a preferred embodiment, the radicals AK2 are not substituted with —OR.

According to a preferred embodiment, AK3 represents a divalent linear or branched, saturated or unsaturated C₁-C₆, more preferentially saturated C₁-C₆, even more preferentially saturated C₁-C₄, better saturated C₃ hydrocarbon-based chain.

According to this embodiment variant, the compound(s) of formula (I) are in particular chosen from the compounds (10) hereinbelow, and also the optical or geometrical isomers thereof and/or the solvates thereof, such as hydrates:

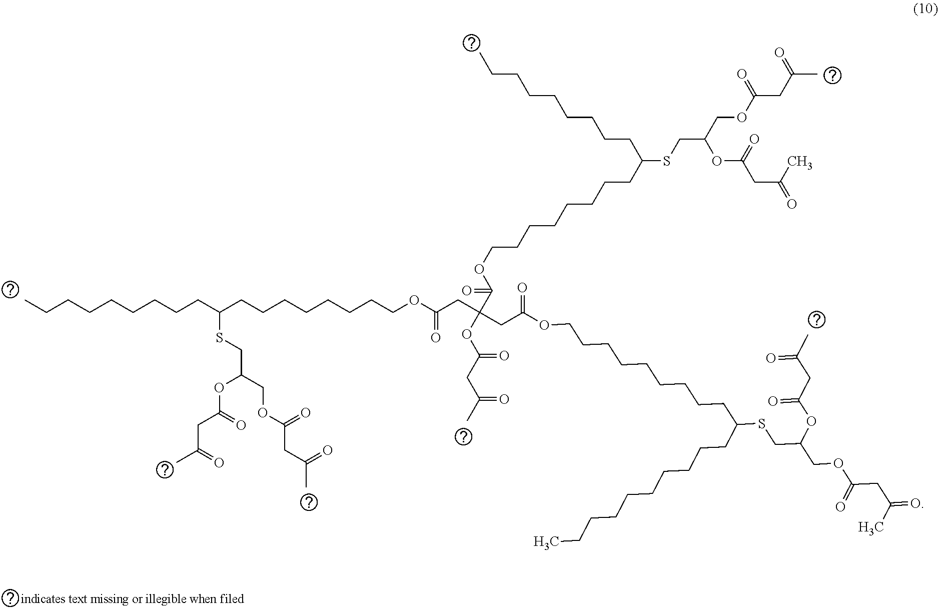 [SMILES: CCCCCCCCCCC(CCCCCCCCCOC(=O)CC(CC(=O)OCCCCCCCCC(CCCCCCCCC)SCC(COC(=O)CC(C)=O)OC(=O)CC(C)=O)(OC(=O)CC(C)=O)C(=O)OCCCCCCCCC(CCCCCCCCC)SCC(COC(=O)CC(C)=O)OC(=O)CC(C)=O)SCC(COC(=O)CC(C)=O)OC(=O)CC(C)=O]

According to a fourth embodiment variant, the compound(s) of formula (I), and also the optical or geometrical isomers and/or solvates thereof, such as hydrates, are such that:
p is equal to 1;
Z represents a linear or branched, saturated or unsaturated, preferably saturated and acyclic, multivalent C₁₄ to C₂₄, particularly C₁₆ to C₂₀, more particularly C₁₇, hydrocarbon-based radical, Z also being optionally substituted with one or more radicals —[(O)ₜ-(AK1)_{q}—(O—C(O)—C(Rₐ)(R_{b})—C(O)—R₄)ᵤ]ₙ, preferably being unsubstituted;
AK2 represents a linear or branched, saturated or unsaturated, preferably saturated, C₁₄-C₃₀, in particular C₂₀-C₂₉, monovalent hydrocarbon-based chain, substituted with one or more identical or different radicals chosen from —OR, —X-AK3-(O—C(O)—C(Rₐ)(R_{b})—C(O)—R₄)ᵥ, preferably substituted with one or more radicals —OR; and/or said chain being optionally interrupted with one or more heteroatoms or groups chosen from —O—, —S—, —C(O)—, (hetero)cycle(s), and combinations thereof, in particular —O—C(O)—, —C(O)—O—, -(hetero)cycle-O-(hetero)cycle-, and preferably being interrupted with —O—C(O)— or —C(O)—O—;
X is as defined previously for formula (I); preferably, X denotes a sulfur atom;
Y, AK1, AK3, n, m, v, t, q, u, R, R₄, Rₐ and R_{b} being as defined previously for formula (I);
   it being understood that said compound contains at least two radicals —C(O)—C(Rₐ)(R_{b})—C(O)—R₄.

According to a preferred embodiment, n is equal to 0 and m is equal to 1.

According to a preferred embodiment, Z is a divalent radical.

According to a preferred embodiment, if Z is an unsaturated chain, AK2 is an unsaturated chain, and if Z is a saturated chain, AK2 is a saturated chain.

According to this embodiment variant, the compound(s) of formula (I), and also the optical or geometrical isomers thereof and/or the solvates thereof, such as hydrates, are in particular of formula (Iic):

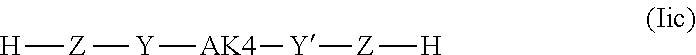

in which formula (Iic):
Y is as defined previously for formula (I);
Z represents a linear or branched, saturated or unsaturated, preferably saturated, C₁₄ to C₂₄, in particular C₁₆ to C₂₀, preferably C₁₇, divalent hydrocarbon-based radical;
Y′ represents —C(O)—O— or —O—C(O)— it being understood that if Y represents —O—C(O)—, Y′ represents —C(O)—O—, and if Y represents —C(O)—O—, Y′ represents —O—C(O)—;
AK4 denotes a saturated divalent hydrocarbon-based radical of 5 or 6 carbon atoms, substituted with at least two radicals —OR with R being as defined previously in formula (I);
   it being understood that the compounds of formula (Iic) contain at least two radicals —C(O)—C(Rₐ)(R_{b})—C(O)—R₄, and preferably four radicals —C(O)—C(Rₐ)(R_{b})—C(O)—R₄.

According to this embodiment variant, the compound(s) of formula (I) are in particular chosen from the compounds (12) hereinbelow, and also the optical or geometrical isomers thereof and/or the solvates thereof, such as hydrates:

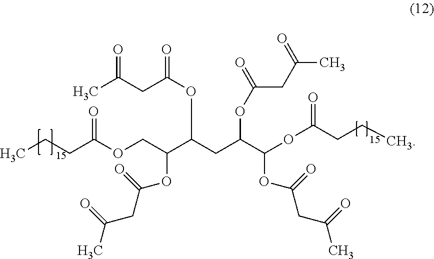

According to a fifth embodiment variant, the compound(s) of formula (I), and also the optical or geometrical isomers and/or solvates thereof, such as hydrates, are such that:
p is equal to 0;
Z represents a linear or branched, saturated or unsaturated, preferably saturated and acyclic, C₂ to C₂₄, in particular C₃ to C₁₀, preferably C₄ or C₅, tetravalent hydrocarbon-based radical, Z also being substituted with one or more radicals —[(O)ₜ-(AK1)_{q}—(O—C(O)—C(Rₐ)(R_{b})—C(O)—R₄)ᵤ]ₙ, n preferably being equal to 1;
n+m is equal to 4, and preferably m is equal to 3;
AK2 independently represents a linear or branched, saturated or unsaturated, preferably saturated, C₁₄ to C₃₀, preferably C₁₆ to C₂₄, preferably C₁₇, hydrocarbon-based chain, said chain being optionally substituted with one or more identical or different radicals chosen from —OR, —X-AK3-(O—C(O)—C(Rₐ)(R_{b})—C(O)—R₄)ᵥ, preferably substituted with one or more radicals —OR; and/or said chain being optionally interrupted with one or more heteroatoms or groups chosen from —O—, —S—, —C(O)—, (hetero)cycle(s), and combinations thereof, in particular —O—C(O)—, —C(O)—O—, -(hetero)cycle-O-(hetero)cycle-, and preferably not being interrupted;
X is as defined previously for formula (I); preferably, X denotes a sulfur atom;
Y, AK1, AK3, u, t, q, v, n, m R, R₄, Rₐ and R_{b} being as defined previously for formula (I);
   it being understood that said compound contains at least two radicals —C(O)—C(Rₐ)(R_{b})—C(O)—R₄.

According to a preferred embodiment, t is equal to 0 and u is equal to 1.

According to a preferred embodiment, all the radicals AK2 are identical, and preferably all the radicals Y are identical.

According to a preferred embodiment, Y denotes —O—C(O)—.

According to a particular embodiment, the compounds according to this variant are of general formula (Iid), and also the optical or geometrical isomers thereof and/or the solvates thereof such as hydrates:

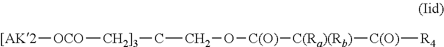

in which formula (Iid) AK′2 represents a linear or branched, preferably linear, saturated or unsaturated, preferably saturated, C₁₄-C₃₀, preferably C₁₆-C₂₄, preferably C₁₇, monovalent hydrocarbon-based chain, said chain being substituted with one or more identical or different radicals chosen from —OR, —S-AK3-(O—C(O)—C(Rₐ)(R_{b})—C(O)—R₄)ᵥ, preferably substituted with one or more radicals —OR; AK3, v, R, R₄, Rₐ and R_{b} being as defined previously for formula (I), it being understood that said compounds contain at least two radicals C(O)—C(Rₐ)(R_{b})—C(O)—R₄.

According to a sixth embodiment variant, the compound(s) of formula (I), and also the optical or geometrical isomers and/or solvates thereof, such as hydrates, are such that:
p is equal to 0;
m is non-zero;
Z represents a linear or branched, saturated or unsaturated, conjugated or non-conjugated, preferably non-conjugated, acyclic or cyclic, aromatic or non-aromatic, preferably non-aromatic, divalent C₂ to C₂₄, in particular C₃ to C₁₂, hydrocarbon-based radical, said radical Z also being interrupted with one or more heteroatoms or groups chosen from —O—, —N(Rₐ)—, —S—, —C(O)—, 5- or 6-membered heterocycloalkyl(s), and combinations thereof;
AK2 represents a linear or branched, saturated or unsaturated, preferably saturated, C₁₄-C₃₀, preferably C₁₄-C₂₄, preferably C₁₇, monovalent hydrocarbon-based chain, said chain being optionally substituted with one or more identical or different radicals chosen from —OR and —X-AK3-(O—C(O)—C(Rₐ)(R_{b})—C(O)—R₄)ᵥ, preferably unsubstituted; and/or said chain being optionally interrupted with one or more heteroatoms or groups chosen from —O—, —S—, —C(O)—, (hetero)cycles, and combinations thereof, in particular —O—C(O)—, —C(O)—O— or -(hetero)cycle-O-(hetero)cycle-, preferably optionally interrupted with —O—C(O)— or —C(O)—O—, and more preferentially not being interrupted;
X is as defined previously for formula (I); preferably, X denotes a sulfur atom;
Y, AK1, AK3, v, t, q, u, R, R₄, Rₐ and R_{b} being as defined previously for formula (I);
   it being understood that said compound contains at least two radicals —C(O)—C(Rₐ)(R_{b})—C(O)—R₄.

According to a preferred embodiment, according to this variant, Z denotes a divalent radical —(CH₂)_{g}-(heterocycle)-O—(CH₂)ₕ-(heterocycle)-(CH₂)ⱼ— in which h is equal to 0, 1 or 2, preferably is equal to 0 or 1, g and j independently denote an integer between 1 and 3, preferably 1, (heterocycle) denotes a divalent 5- or 6-membered heterocycloalkyl radical, in particular a divalent tetrahydrofuran radical or a divalent tetrahydropyran radical, said heterocycle being optionally substituted with one or more radicals chosen from —OR, with R as defined previously for formula (I), and notably —OH or —O—C(O)—C(Rₐ)(R_{b})—C(O)—R₄.

According to a particular embodiment, the compounds according to this variant are of general formula (lie), and also the optical or geometrical isomers thereof and/or the solvates thereof such as hydrates:

AK″2-C(O)—O—(CH₂)_{g}-(heterocycle)-O—(CH₂)ₕ-(heterocycle)-(CH₂)ⱼ—O—CO-AK″2 (Iie)

in which formula (Iie):
h is equal to 0, 1 or 2 and preferably is equal to 0;
g and j independently denote an integer between 1 and 3, preferably 1;
(heterocycle) denotes a divalent 5- or 6-membered heterocycloalkyl radical substituted with one or more identical or different radicals chosen from —OR, with R as defined previously for formula (I), and notably —OH or —O—C(O)—C(Rₐ)(R_{b})—C(O)—R₄;
AK″2 represents a linear or branched, saturated or unsaturated, preferably saturated, C₁₄-C₃₀, preferably C₁₄-C₂₄, preferably C₁₇, monovalent hydrocarbon-based chain, said chain being optionally substituted with one or more identical or different radicals chosen from —OH, —O—C(O)—C(Rₐ)(R_{b})—C(O)—R₄ and —S-AK3-(O—C(O)—C(Rₐ)(R_{b})—C(O)—R₄)ᵥ, preferably unsubstituted; and/or said chain being optionally interrupted with one or more heteroatoms or groups chosen from —O—, —S—, —C(O)—, (hetero)cycle(s), and combinations thereof, in particular —O—C(O)—, —C(O)—O— or -(hetero)cycle-O-(hetero)cycle-, preferably optionally interrupted with —O—C(O)— or —C(O)—O—, and more preferentially not being interrupted;
   it being understood that said compound contains at least two radicals —C(O)—C(Rₐ)(R_{b})—C(O)—R₄.

According to this embodiment variant, the compounds of formula (I) or (Iie) are in particular chosen from the compounds (14) hereinbelow, and also the optical and geometrical isomers thereof and/or the solvates thereof, such as hydrates:

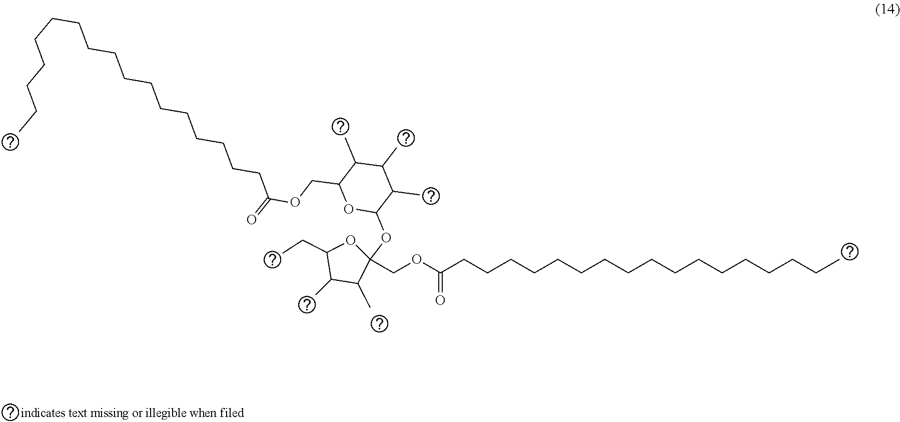 [SMILES: CCCCCCCCCCCCCCCCCC(=O)OCC1OC(OC2(COC(=O)CCCCCCCCCCCCCCCCC)OC(CC)C(C)C2C)C(C)C(C)C1C]

with W being a hydrogen atom or —C(O)—C(Rₐ)(R_{b})—C(O)—R₄, and said compound containing at least two groups —C(O)—C(Rₐ)(R_{b})—C(O)—R₄.

According to a seventh embodiment variant, the compound(s) of formula (I), and also the optical or geometrical isomers and/or solvates thereof, such as hydrates, are such that:
p is equal to 0;
Z represents a linear or branched, saturated or unsaturated, preferably saturated and acyclic, C2 to C24, in particular C3 to C10, preferably C4 or C5, tetravalent hydrocarbon-based radical, Z also being substituted with one or more radicals —[(O)t-(AK1)q-(O—C(O)—C(Ra)(Rb)—C(O)—R4)u]n;
n+m is equal to 6, and preferably m is equal to 2;
AK2 represents a linear or branched, saturated or unsaturated, preferably saturated, C₃-C₈, in particular C₄, C₅ or C₆, monovalent hydrocarbon-based chain;
Y, AK1, AK3, t, q, u, v, R, X, R₄, Rₐ and R_{b} being as defined previously for formula (I);
   it being understood that said compound contains at least two radicals —C(O)—C(Rₐ)(R_{b})—C(O)—R₄.

According to this embodiment variant, the compounds of formula (I) are in particular chosen from the compound (15) hereinbelow, and also the optical and geometrical isomers thereof and/or the solvates thereof, such as hydrates:

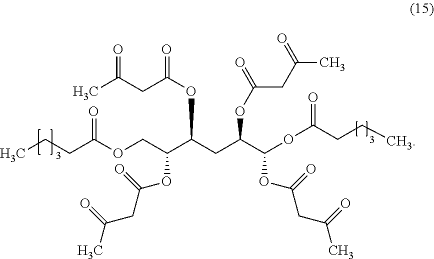

According to a preferred embodiment, the compound(s) of formula (I) are chosen from compounds (1) to (15) below, and also the optical or geometrical isomers thereof, and/or the solvates thereof, such as hydrates:

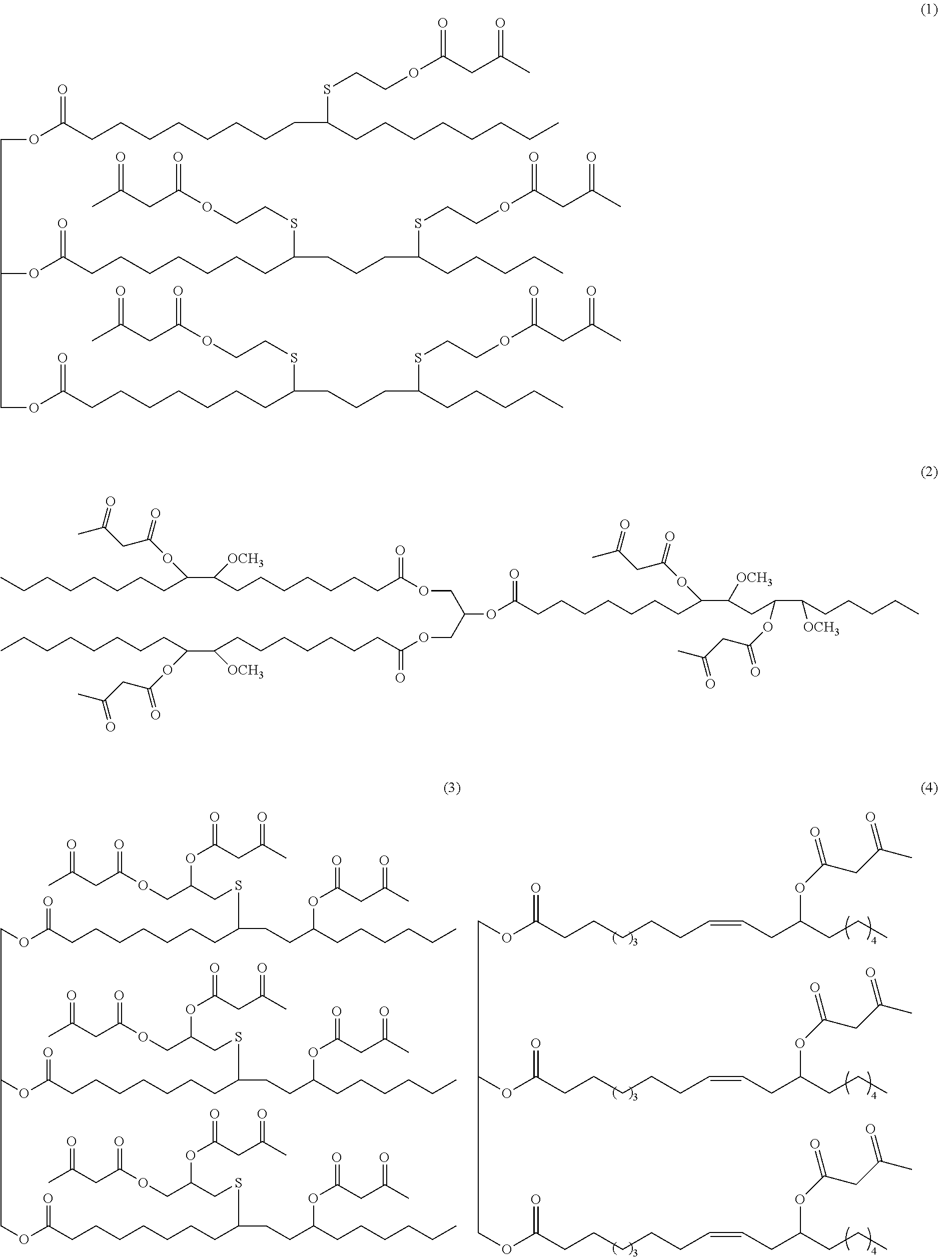

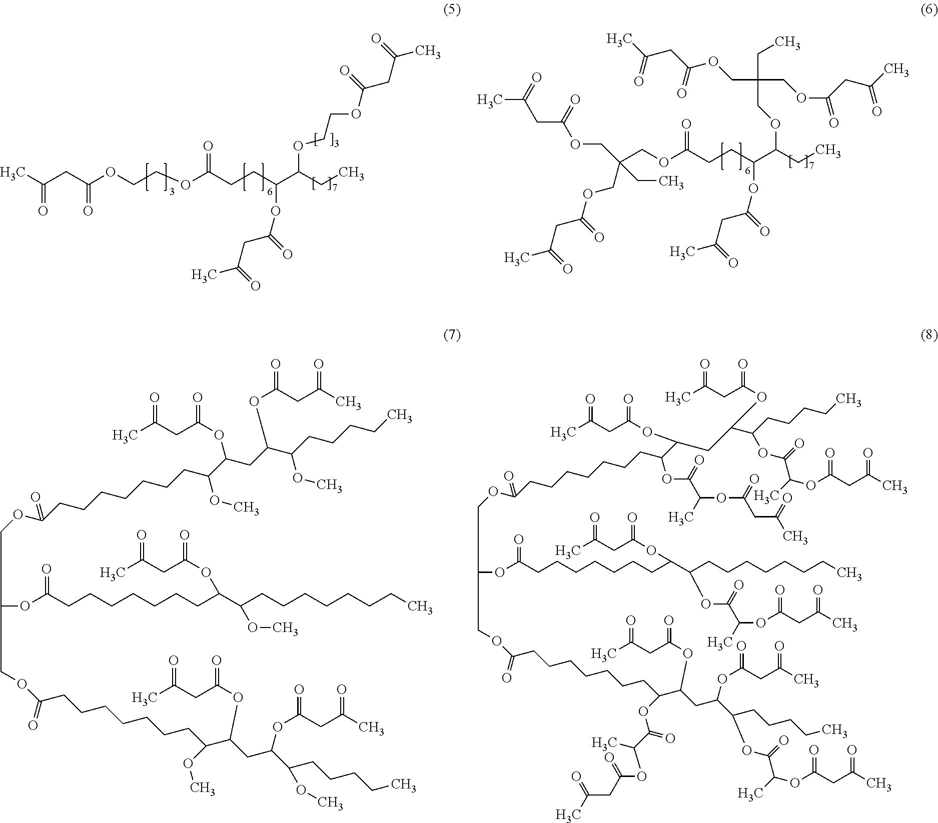

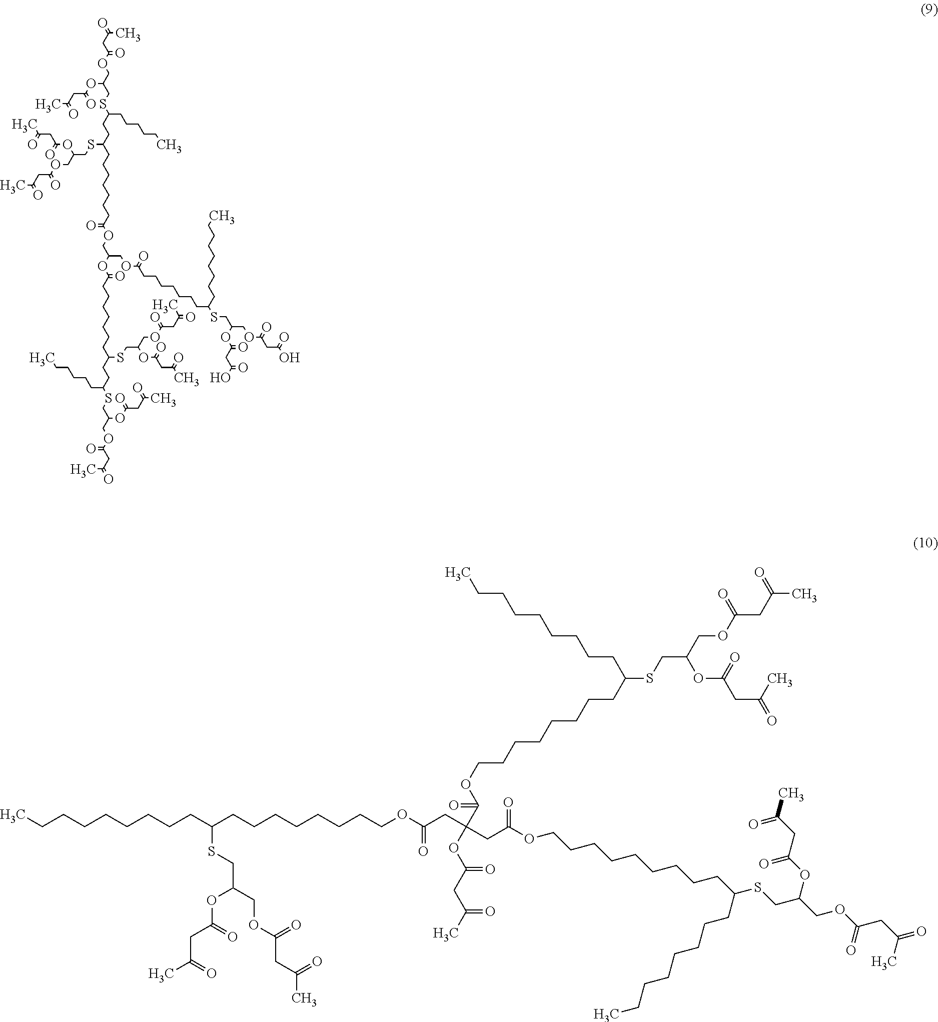

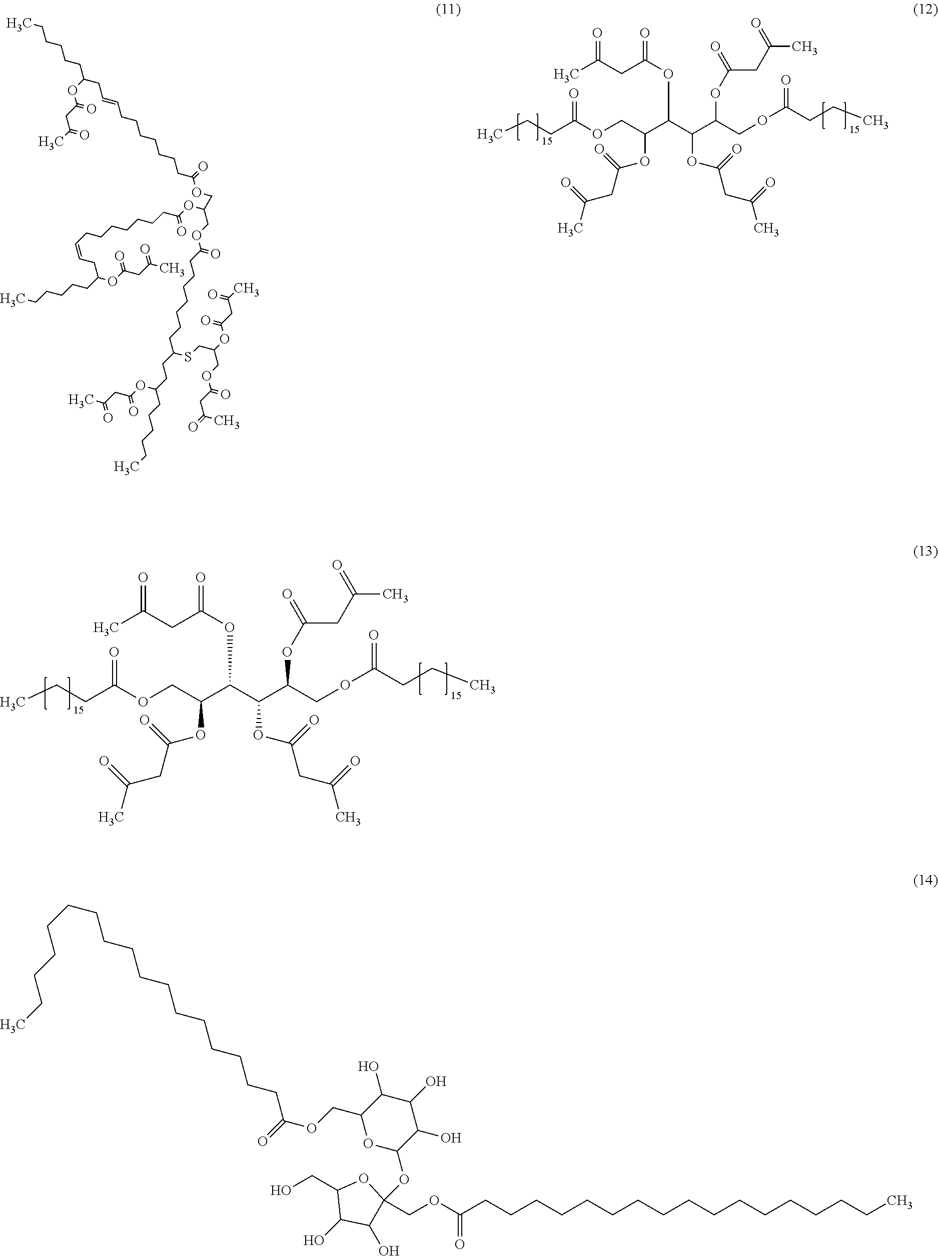

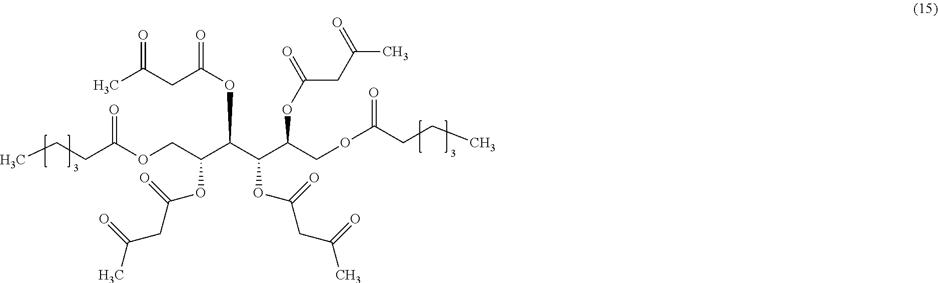

with W being a hydrogen atom or —C(O)—C(Rₐ)(R_{b})—C(O)—R₄, and said compound containing at least two groups —C(O)—C(Rₐ)(R_{b})—C(O)—R₄.

According to a preferred embodiment, the compound(s) of formula (I) are chosen from compounds (1) to (14) above, and also the optical or geometrical isomers thereof, and/or the solvates thereof, such as hydrates.

According to a preferred embodiment, the compound(s) of formula (I) are in particular chosen from compounds (1), (3), (4), (8), (9), (10), (11) and (15) hereinbelow, and also the optical or geometrical isomers thereof and/or the solvates thereof, such as hydrates:

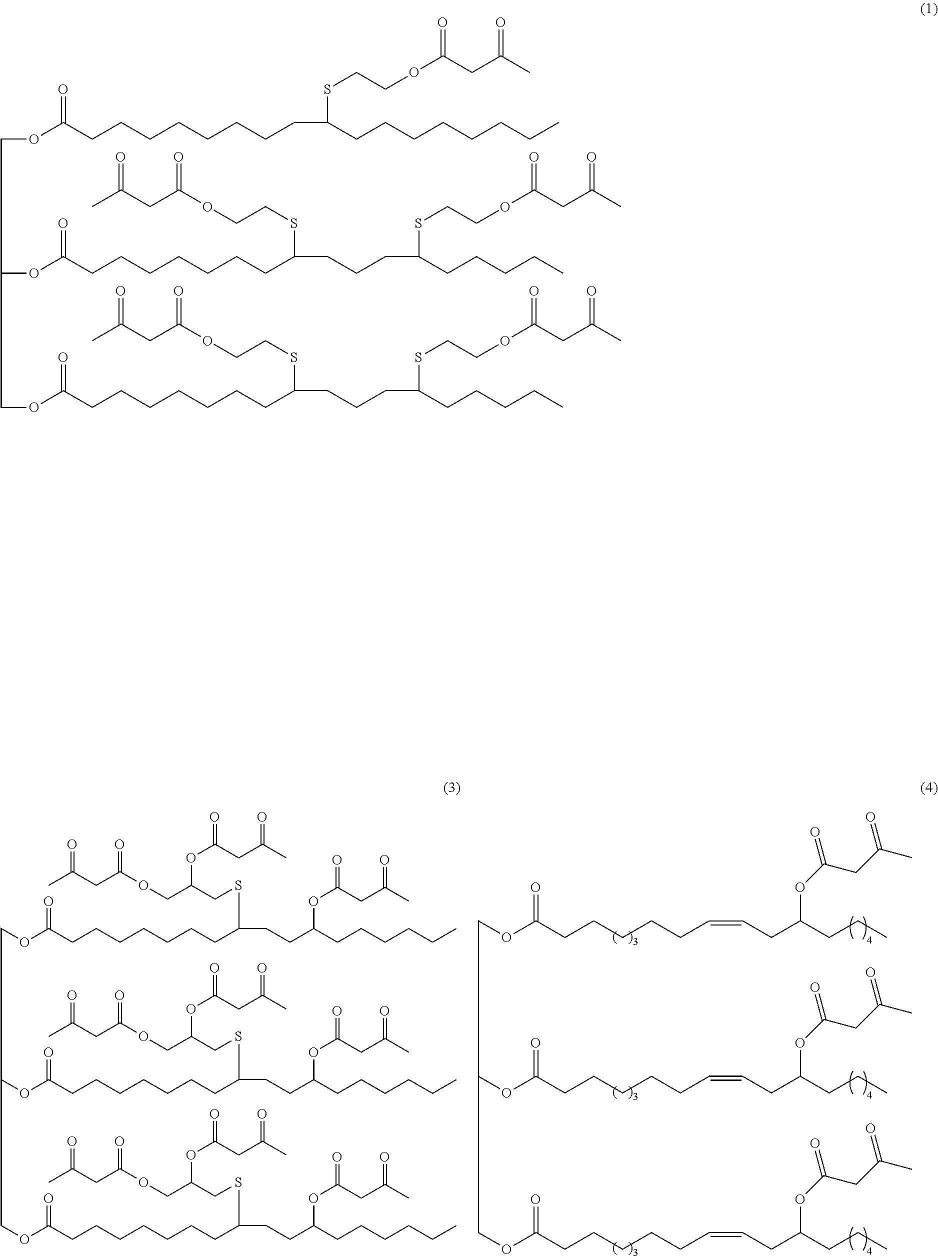

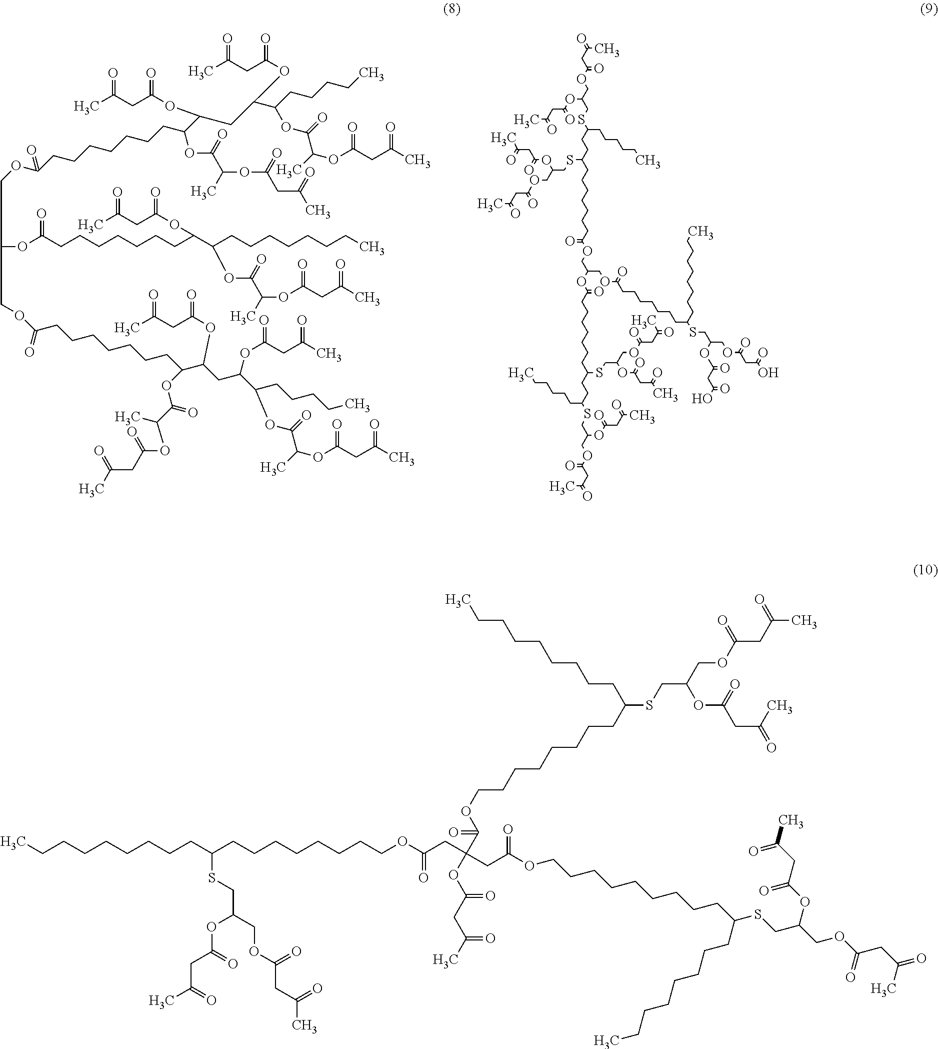

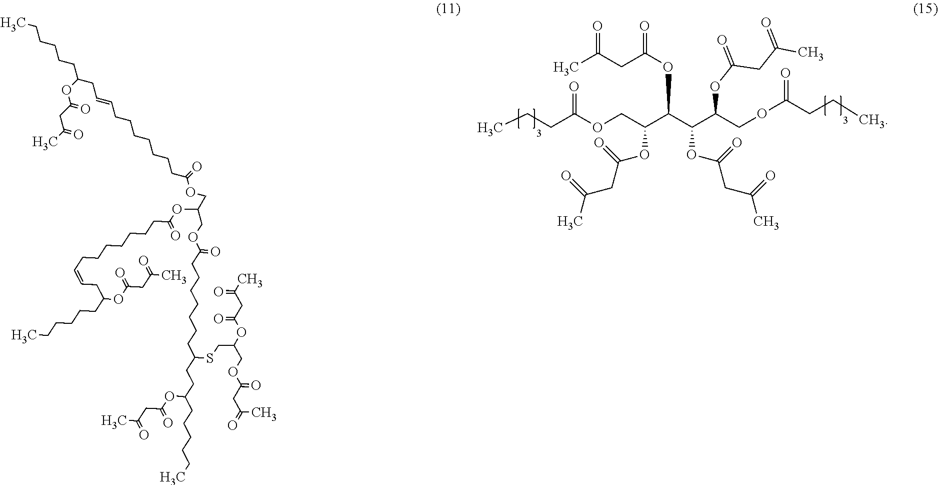

According to a preferred embodiment, the compound(s) of formula (I) are in particular chosen from compounds (1), (3), (4), (8), (9), (10) and (11) above, and also the optical or geometrical isomers thereof and/or the solvates thereof, such as hydrates.

According to a particular embodiment, the treatment process according to the invention comprises the application to keratin materials, notably the skin and/or the lips and/or the eyelashes and/or the eyebrows, of at least one compound of formula (I), and also the optical or geometrical isomers thereof and/or the solvates thereof, such as hydrates, as defined hereinabove, or a composition containing same, with the exception of the compounds:

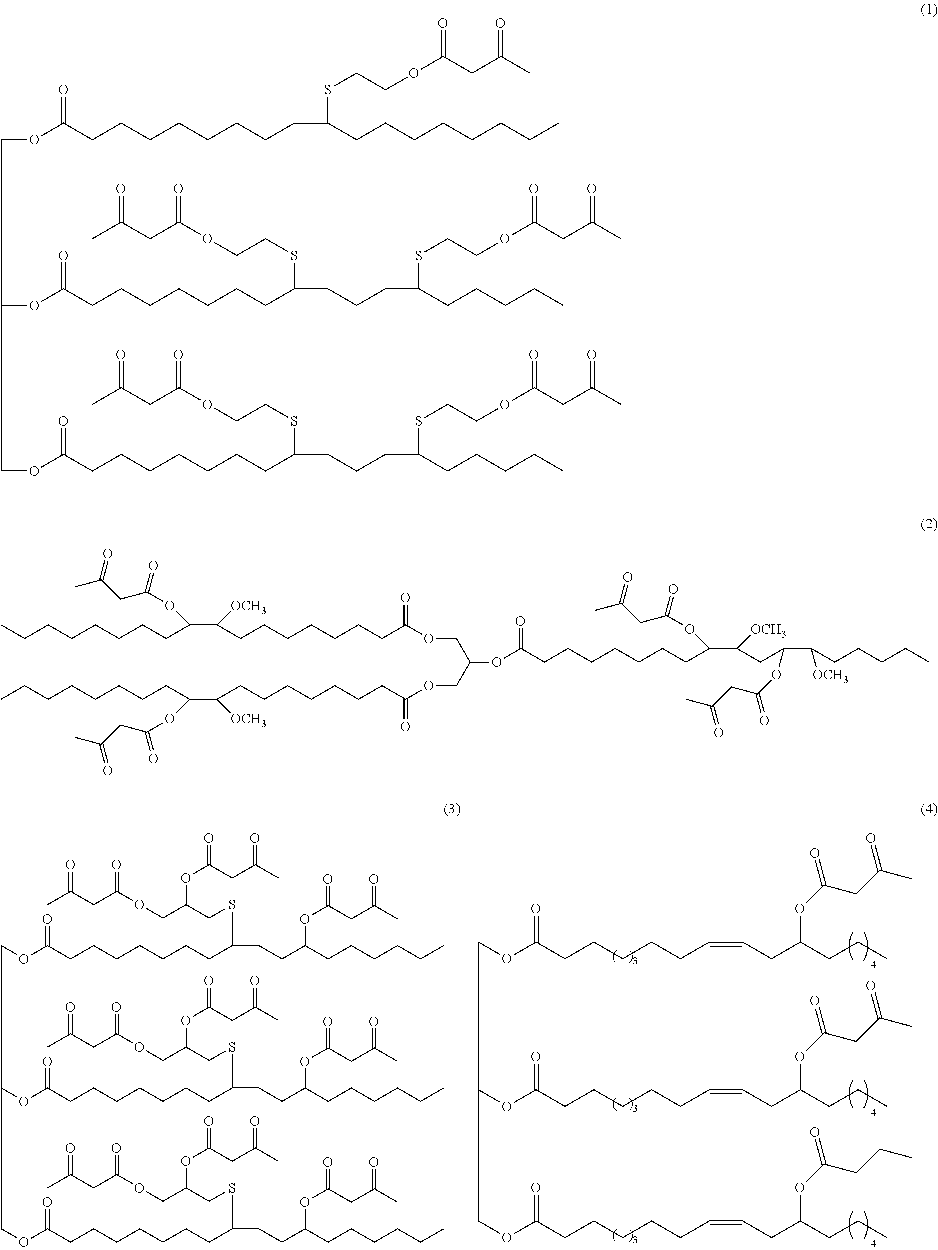

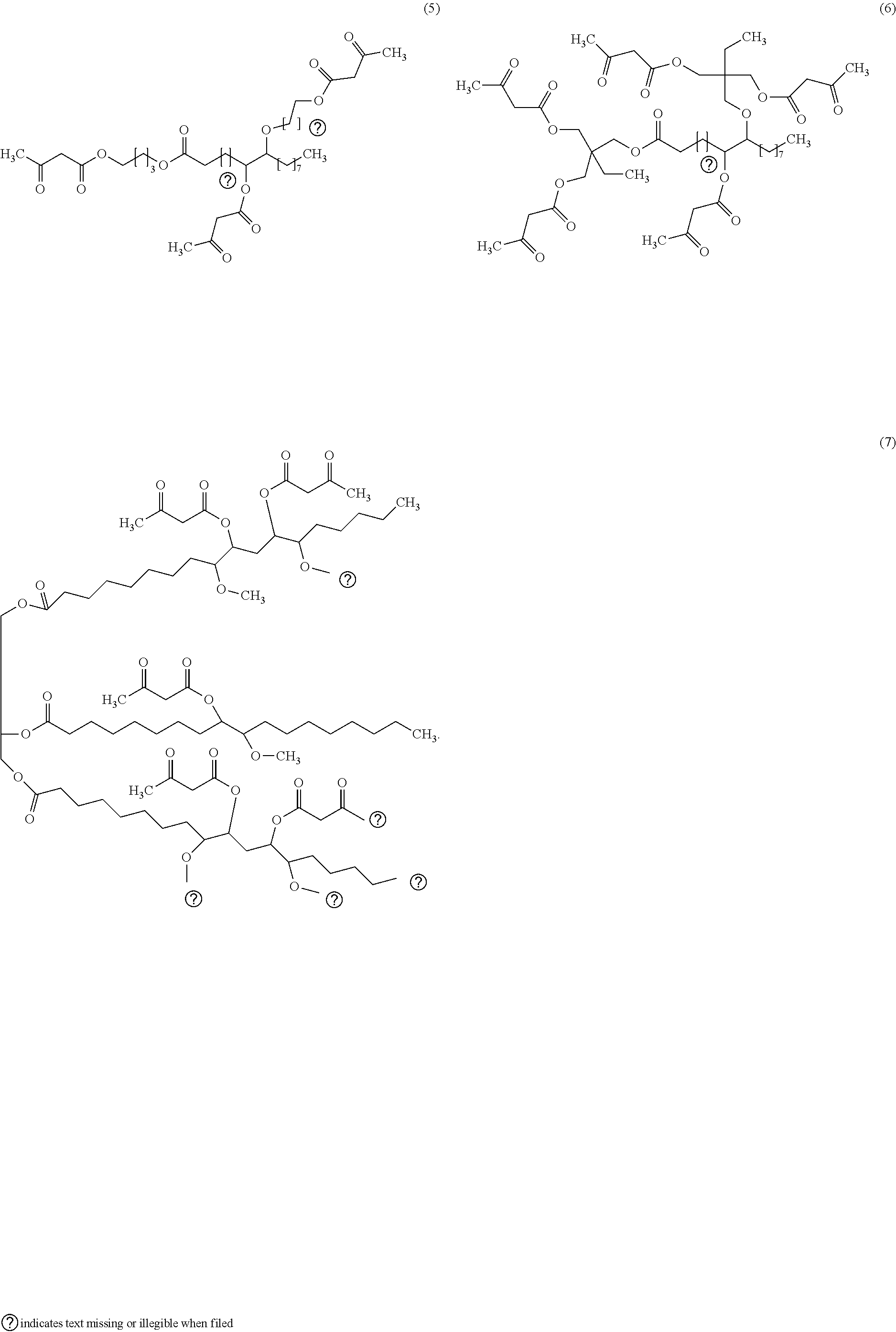

According to a particular embodiment, the treatment process according to the invention comprises the application to keratin materials, notably the skin and/or the lips and/or the eyelashes and/or the eyebrows, of at least one compound of formula (I), and also the optical or geometrical isomers thereof and/or the solvates thereof, such as the hydrates, or a composition containing same, chosen from compounds (8), (9), (10), (11), (12), (13) and (14) below, and also the optical or geometrical isomers thereof and/or the solvates thereof, such as the hydrates:

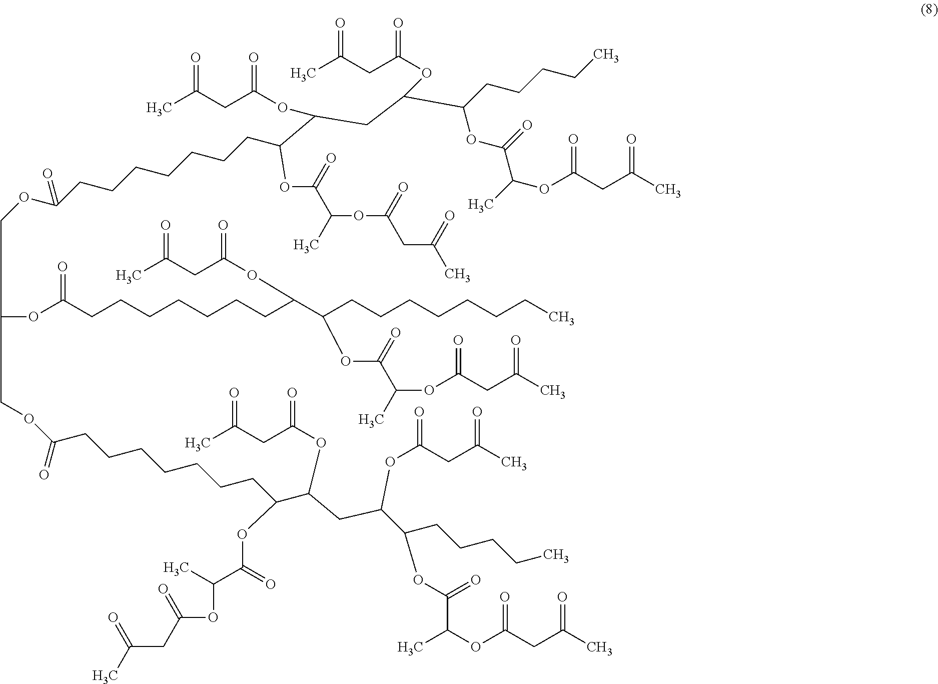 [SMILES: CCCCCCCCC(OC(=O)C(C)OC(=O)CC(C)=O)C(CCCCCCCC(=O)OC(COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)OC(=O)CC(C)=O]

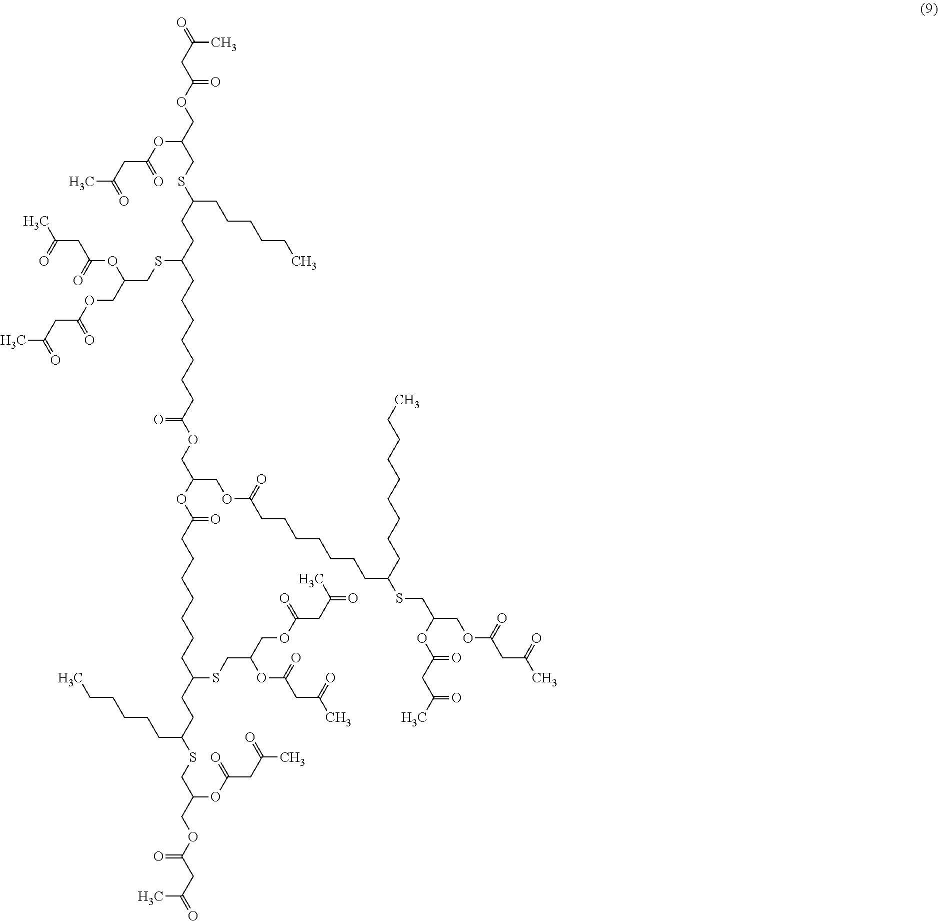 [SMILES: CCCCCCCCCC(CCCCCCCC(=O)OCC(COC(=O)CCCCCCCC(CCC(CCCCCC)SCC(COC(=O)CC(C)=O)OC(=O)CC(C)=O)SCC(COC(=O)CC(C)=O)OC(=O)CC(C)=O)OC(=O)CCCCCCCC(CCC(CCCCCC)SCC(COC(=O)CC(C)=O)OC(=O)CC(C)=O)SCC(COC(=O)CC(C)=O)OC(=O)CC(C)=O)SCC(COC(=O)CC(C)=O)OC(=O)CC(C)=O]

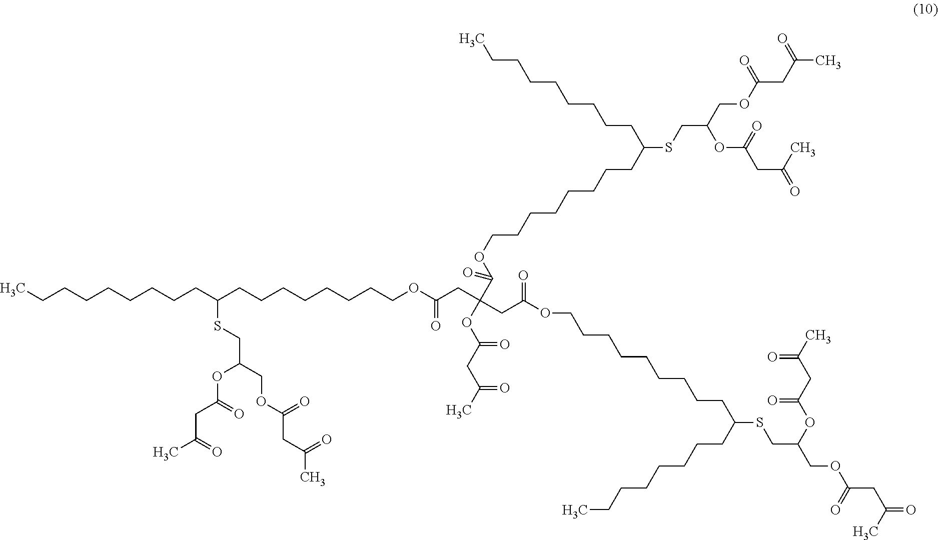 [SMILES: CCCCCCCCCC(CCCCCCCCOC(=O)CC(CC(=O)OCCCCCCCCCC(CCCCCCCC)SCC(COC(=O)CC(C)=O)OC(=O)CC(C)=O)(OC(=O)CC(C)=O)C(=O)OCCCCCCCCC(CCCCCCCCC)SCC(COC(=O)CC(C)=O)OC(=O)CC(C)=O)SCC(COC(=O)CC(C)=O)OC(=O)CC(C)=O]

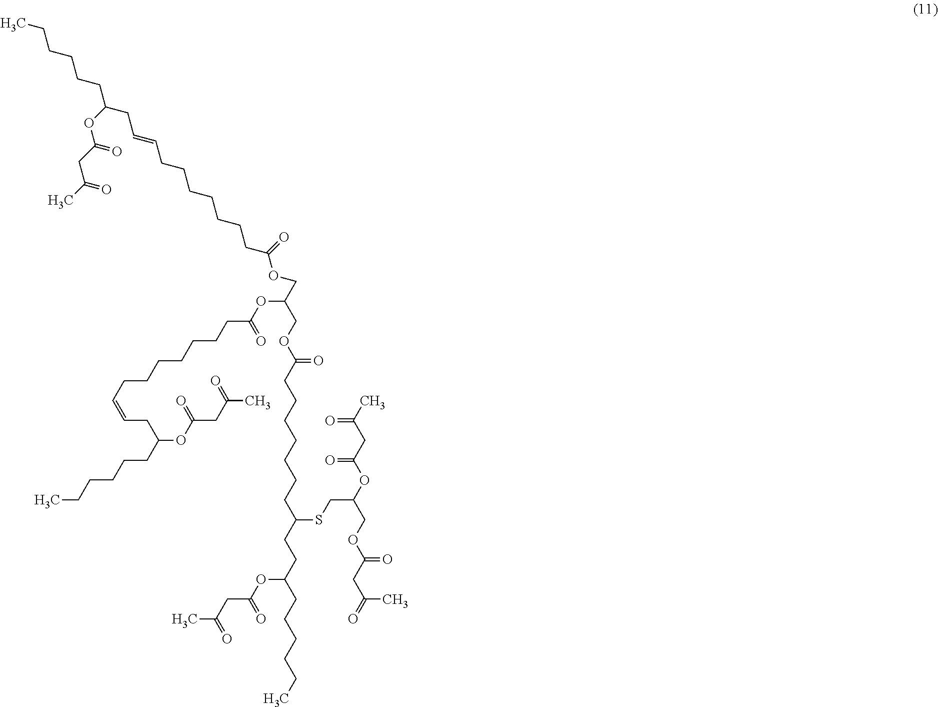 [SMILES: CCCCCCC(C/C=C\CCCCCCCC(=O)OC(COC(=O)CCCCCCC/C=C/CC(CCCCCC)OC(=O)CC(C)=O)COC(=O)CCCCCCCC(CCC(CCCCCC)OC(=O)CC(C)=O)SCC(COC(=O)CC(C)=O)OC(=O)CC(C)=O)OC(=O)CC(C)=O]

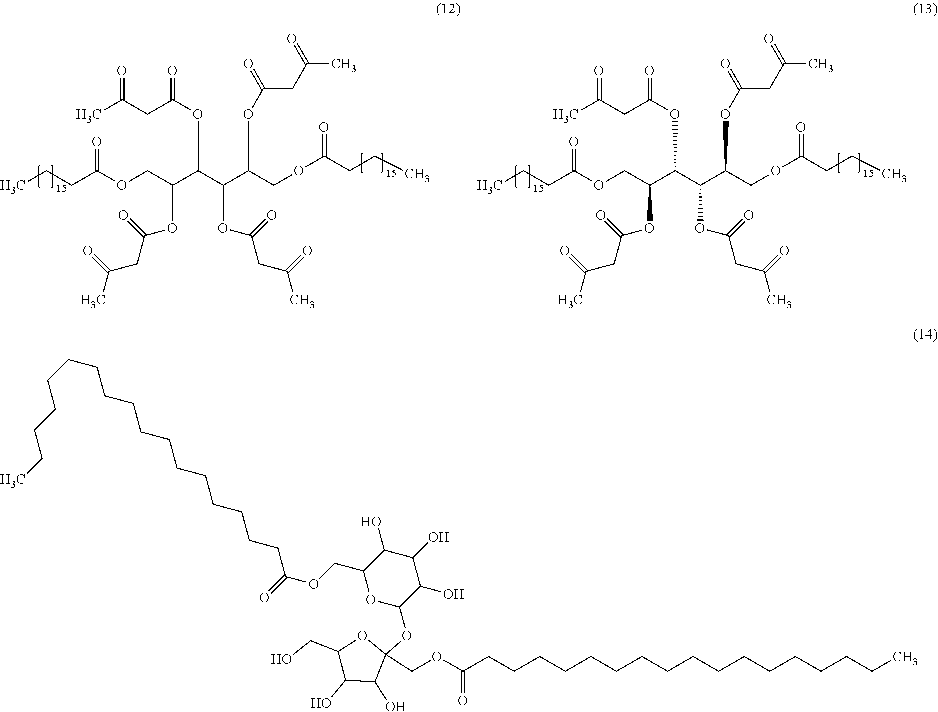

with W being a hydrogen atom or —C(O)—C(Rₐ)(R_{b})—C(O)—R₄, and said compound containing at least two groups —C(O)—C(Rₐ)(R_{b})—C(O)—R₄.

According to a particular embodiment, the treatment process according to the invention comprises the application to keratin materials, notably to the skin and/or the lips and/or the eyelashes and/or the eyebrows, of at least one compound of formula (I), and also the optical or geometrical isomers thereof and/or the solvates thereof, such as hydrates, or a composition containing same, chosen from the following compounds of formula (Ia), and also the optical or geometrical isomers thereof and/or the solvates thereof, such as hydrates:

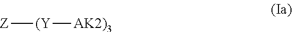

in which formula (Ia):
Z represents —CH₂—CH—CH₂—;
AK2 independently denotes a linear or branched, saturated or unsaturated, C₁₂-C₂₄ monovalent hydrocarbon-based chain, optionally substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, epoxides, and combinations thereof, in particular O—C(O)— and —C(O)—O—; —Y, AK3, v, R, X, R₄, Rₐ and R_{b} being as defined previously in formula (I); preferably, Y denotes —O—C(O);
   it being understood that said compound contains at least two radicals —C(O)—C(Rₐ)(R_{b})—C(O)—R₄;
   in particular with the exception of the compounds (1), (2), (3), (4) and (7) as defined previously, and also the optical or geometrical isomers thereof and/or the solvates thereof, such as hydrates.

According to a particular embodiment, the compound(s) of formula (Ia) are in particular chosen from compounds (8), (9) and (11), as defined previously, and also the optical or geometrical isomers thereof and/or the solvates thereof, such as hydrates.

According to a particular embodiment, the compound(s) of formula (Ia), and also the optical and geometrical isomers thereof and/or the solvates thereof, such as hydrates, are compounds for which:
a) at least one of the three radicals AK2 denotes a branched, saturated or unsaturated C₁₂-C₂₄ monovalent hydrocarbon-based chain optionally substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), in particular epoxides, and combinations thereof, in particular —O—C(O)— or —C(O)—O—; AK3, v, R, X, R₄, Rₐ and R_{b} being as defined previously; it being understood that said compound contains at least two radicals C(O)—C(Rₐ)(R_{b})—C(O)—R₄; preferably Y represents —O—C(O); preferably X represents S; preferably the three chains AK2 contain from 15 to 21 carbon atoms, more preferably 17 carbon atoms; and preferably none of the three radicals AK2 is interrupted; or
b) the three radicals AK2 independently represent a linear, saturated, C₁₂-C₂₄ hydrocarbon-based chain substituted with at least one radical chosen from —OH and OR′, and optionally substituted with one or more identical or different radicals chosen from —O—C(O)—C(Rₐ)(R_{b})—C(O)—R₄ and —X′-AK3-(O—C(O)—C(Rₐ)(R_{b})—C(O)—R₄)ᵥ; R′ denoting a C₂-C₆ alkyl radical, in particular ethyl; AK3, v, R, R₄, Rₐ and R_{b} being as defined previously, preferably, Y denotes —O—C(O)—; and X′ represents —O— or —C(O)—O—; it being understood that said compound contains at least two radicals —C(O)—C(Rₐ)(R_{b})—C(O)—R₄; preferably, the three chains AK2 comprise from 15 to 21 carbon atoms, more preferably 17 carbon atoms; and preferably none of the three radicals AK2 is interrupted; or
c) the radicals AK2 independently represent a linear, saturated, C₁₂-C₂₄ monovalent hydrocarbon-based chain, optionally substituted with one or more identical or different radicals chosen from —OR and —X-AK3-(O—C(O)—C(Rₐ)(R_{b})—C(O)—R₄)ᵥ; said chain being interrupted with one or more heteroatoms or groups chosen from —O— and/or —S— and/or —C(O)—, (hetero)cycle(s), in particular one or more epoxides, and combinations thereof, preferably —O—C(O)—, —C(O)—O— and -(hetero)cycle-O-(hetero)cycle-; AK3, v, R, X, R₄, Rₐ and R_{b} being as defined previously; and it being understood that said compound contains at least two radicals C(O)—C(Rₐ)(R_{b})—C(O)—R₄; preferably Y represents —O—C(O)—; preferably, X represents S; preferably, the three chains AK2 comprise from 15 to 21 carbon atoms, more preferentially 17 carbon atoms; or
d) the three radicals AK2 are such that at least one of the three radicals AK2 represents a linear or branched, unsaturated C₁₂-C₂₄ monovalent hydrocarbon-based chain, optionally substituted with one or more identical or different radicals chosen from —OR, said chain optionally being interrupted with one or more heteroatoms or groups chosen from —O—, —S—, —C(O)—, (hetero)cycle(s), and combinations thereof, in particular —O—C(O)—, —C(O)—O— or (hetero)cycle-O-(hetero)cycle; and at least one of the three radicals AK2 represents a linear or branched, saturated C₁₂-C₂₄ monovalent hydrocarbon-based chain, substituted with one or more identical or different radicals chosen from —S-AK3-(O—C(O)—C(Rₐ)(R_{b})—C(O)—R₄)ᵥ and optionally substituted with one or more identical or different radicals chosen from —OR; AK3, v, R, R₄, Rₐ and R_{b} being as defined previously; preferably, Y denotes —O—C(O)—; it being understood that said compound contains at least two radicals —C(O)—C(Rₐ)(R_{b})—C(O)—R₄; preferably, Y represents —O—C(O)—; preferably, X represents S; preferably, the three chains AK2 comprise from 15 to 21 carbon atoms, more preferentially 17 carbon atoms; preferably, none of the three radicals AK2 is interrupted; or
e) the three radicals AK2 are such that AK2 represents a saturated linear monovalent C₁₂-C₂₄ hydrocarbon-based chain substituted with one or more identical or different radicals chosen from —S-AK3-(O—C(O)—C(Rₐ)(R_{b})—C(O)—R₄)ᵥ; said chain being optionally interrupted with one or more heteroatoms or groups chosen from —O—, —S—, —C(O)—, (hetero)cycle(s), and combinations thereof, such as —O—C(O)—, —C(O)—O—, (hetero)cycle-O-(hetero)cycle; AK3 represents a divalent, linear or branched, saturated or unsaturated C₃-C₁₂, preferably C₃-C₆, more preferentially saturated C₃-C₆, in particular C₃ hydrocarbon-based chain; v, R₄, Rₐ and R_{b} being as defined previously; preferably, Y denotes —O—C(O)—; it being understood that said compound contains at least two radicals —C(O)—C(Rₐ)(R_{b})—C(O)—R₄; preferably, Y represents —O—C(O)—; preferably, v is equal to 2; preferably, the three chains AK2 comprise from 15 to 21 carbon atoms, more preferentially 17 carbon atoms; preferably, none of the three radicals AK2 is interrupted; preferably, the radicals AK2 are saturated; preferably, AK3 denotes a trivalent radical —CH₂—CH—CH₂—;
   or
f) the three radicals AK2 represent a linear, saturated, C₁₂-C₂₄ monovalent hydrocarbon-based chain substituted with one or more identical or different radicals —O—C(O)-AK3-(O—C(O)—C(Rₐ)(R_{b})—C(O)—R₄)ᵥ, said chain also being optionally substituted with one or more identical or different radicals chosen from —OR and —S-AK3-(O—C(O)—C(Rₐ)(R_{b})—C(O)—R₄)ᵥ and/or said chain being optionally interrupted with one or more heteroatoms or groups chosen from —O—, —S—, —C(O)—, (hetero)cycle(s) and combinations thereof, in particular —O—C(O)—, —C(O)—O— or -(hetero)cycle-O-(hetero)cycle-; AK3, v, R₄, Rₐ and R_{b} being as defined previously; preferably, Y denotes —O—C(O)—; it being understood that said compound contains at least two radicals —C(O)—C(Rₐ)(R_{b})—C(O)—R₄; preferably, Y represents —O—C(O)—; preferably, v is equal to 1 or 2, more preferentially equal to 1; preferably, the three chains AK2 comprise from 15 to 21 carbon atoms, more preferentially 17 carbon atoms; preferably, none of the three radicals AK2 is interrupted; preferably, the radicals AK2 are saturated; preferably, the radicals AK2 are substituted with at least one radical —OR, preferably at least one radical —O—C(O)—C(ₐ)(R_{b})—C(O)—R₄; preferably, AK2 is not substituted with —S-AK3-(O—C(O)—C(Rₐ)(R_{b})—C(O)—R₄)ᵥ; preferably, AK3 represents a linear or branched, saturated or unsaturated C₁-C₆, more preferentially saturated C₁-C₆, even more preferentially C₂, hydrocarbon-based chain.

According to a particular embodiment, the treatment process according to the invention comprises the application to keratin materials, notably the skin and/or the lips and/or the eyelashes and/or the eyebrows, of at least one compound of formula (I), and also the optical or geometrical isomers thereof and/or the solvates thereof, such as the hydrates, or a composition containing same, chosen from the following compounds of formula (Ib), and also the optical or geometrical isomers thereof and/or the solvates thereof, such as the hydrates:

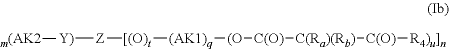

in which formula (Ib):
n+m is equal to 4, m being non-zero; m and n being as defined previously for the compounds of formula (I);
Z represents an unsubstituted and uninterrupted, linear or branched, saturated or unsaturated, preferably saturated and acyclic, C₂ to C₂₄, in particular C₃ to C₁₀ preferably C₃, tetravalent hydrocarbon-based radical;
AK2 independently represent a linear or branched, saturated or unsaturated, preferably saturated, C₁₂-C₂₄, preferably C₁₆-C₂₀, preferably C₁₈, monovalent hydrocarbon-based chain, optionally substituted with one or more identical or different radicals chosen from —OR and —X-AK3-(O—C(O)—C(Rₐ)(R_{b})—C(O)—R₄)ᵥ, preferably substituted with one or more radicals —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), and combinations thereof, in particular —O—C(O)—, —C(O)—O— or -(hetero)cycle-O-(hetero)cycle-, and preferably not being interrupted;
AK3 represents a linear or branched, saturated or unsaturated C₁-C₁₂, preferably C₁-C₆, more preferentially saturated C₁-C₆, even more preferentially C₂-C₄, preferably C₃, divalent hydrocarbon-based chain;
v is as defined previously; preferably, v is equal to 2;
X is as defined previously; preferably, X denotes a sulfur atom;
Y, AK1, u, t, q, R, R₄, Rₐ and R_{b} being as defined previously;
   it being understood that said compound contains at least two radicals —C(O)—C(Rₐ)(R_{b})—C(O)—R₄.

According to a preferred embodiment, m is equal to 3, n is equal to 1 and u is equal to 1.

According to a preferred embodiment, t is equal to 0 and q is equal to 0.

According to a preferred embodiment, Y denotes —C(O)—O—.

According to a preferred embodiment, none of the radicals AK2 are interrupted. Preferably, the radicals AK2 are saturated. Preferably, the radicals AK2 are not substituted with —OR.

According to a preferred embodiment, AK3 represents a linear or branched, saturated or unsaturated C₁-C₆, more preferentially saturated C₁-C₆, even more preferentially C₁-C₄, better still C₃, hydrocarbon-based chain.

According to this embodiment variant, the compounds of formula (I) or (Ib) are in particular chosen from the compounds (10) as defined previously, and also the optical or geometrical isomers thereof and/or the solvates thereof, such as hydrates.

According to a particular embodiment, the treatment process according to the invention comprises the application to keratin materials, notably the skin and/or the lips and/or the eyelashes and/or the eyebrows, of at least one compound of formula (I), and also the optical or geometrical isomers thereof and/or the solvates thereof, such as the hydrates, or a composition containing same, chosen from the following compounds of formula (Ic), and also the optical or geometrical isomers thereof and/or the solvates thereof, such as the hydrates:

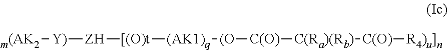

in which formula (Ic):
Z represents a linear or branched, saturated or unsaturated, preferably saturated and acyclic, multivalent C₁₄ to C₂₄, particularly C₁₆ to C₂₀, such as C₁₇, hydrocarbon-based radical, Z also being optionally substituted with one or more radicals —[(O)ₜ-(AK1)_{q}—(O—C(O)—C(Rₐ)(R_{b})—C(O)—R₄)ᵤ]ₙ, preferably unsubstituted;
AK2 represents a linear or branched, saturated or unsaturated, preferably saturated, C₁₄-C₃₀, preferably C₂₀-C₂₉, monovalent hydrocarbon-based chain, substituted with one or more identical or different radicals chosen from —OR and —X-AK3-(O—C(O)—C(Rₐ)(R_{b})—C(O)—R₄)ᵥ, preferably substituted with one or more radicals —OR; and/or said chain being optionally interrupted with one or more heteroatoms or groups chosen from —O—, —S—, —C(O)—, (hetero)cycle(s), and combinations thereof, in particular —O—C(O)—, —C(O)—O—, -(hetero)cycle-O-(hetero)cycle-, and preferably interrupted with —O—C(O)— or —C(O)—O—;
X is as defined previously, and X preferably represents a sulfur atom;
Y, AK1, AK3, n, m, v, t, q, u, R, R₄, Rₐ and R_{b} being as defined previously;
it being understood that said compound contains at least two radicals —C(O)—C(Rₐ)(R_{b})—C(O)—R₄.

According to a preferred embodiment, n is equal to 0 and m is equal to 1.

According to a preferred embodiment, Z is a divalent radical.

According to a preferred embodiment, if Z is an unsaturated chain, AK2 is an unsaturated chain, and if Z is a saturated chain, AK2 is a saturated chain.

According to this embodiment variant, the compound(s) of formula (Ic), and also the optical or geometrical isomers thereof and/or the solvates thereof, such as hydrates, are in particular of formula (Iic) as defined previously.

According to this embodiment variant, the compound(s) of formula (Iic) are in particular chosen from the compounds (12) as defined previously, and also the optical or geometrical isomers thereof and/or the solvates thereof, such as hydrates.

According to a particular embodiment, the treatment process according to the invention comprises the application to keratin materials, notably the skin and/or the lips and/or the eyelashes and/or the eyebrows, of at least one compound of formula (I) and also the optical and geometrical isomers thereof, and/or the solvates thereof, such as the hydrates, or a composition containing same, chosen from the following compounds of formula (Id), and also the optical or geometrical isomers thereof and/or the solvates thereof, such as the hydrates:

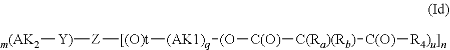

in which formula (Id):
n+m is equal to 4, and preferably m is equal to 3;
Z represents a linear or branched, saturated or unsaturated, preferably saturated or acyclic, C₂ to C₂₄, in particular C₃ to C₁₀, in particular C₄ or C₅, tetravalent hydrocarbon-based radical, Z also being substituted with one or more radicals —[(O)ₜ-(AK1)_{q}—(O—C(O)—C(Rₐ)(R_{b})—C(O)—R₄)ᵤ]ₙ, n preferably being equal to 1;
AK2 independently represents a linear or branched, saturated or unsaturated, preferably saturated, C₁₄-C₃₀, preferably C₁₆-C₂₄, preferably C₁₇, monovalent hydrocarbon-based chain, said chain being optionally substituted with one or more identical or different radicals chosen from —OR and —X-AK3-(O—C(O)—C(Rₐ)(R_{b})—C(O)—R₄)ᵥ, preferably substituted with one or more radicals —OR; and/or said chain being optionally interrupted with one or more heteroatoms or groups chosen from —O—, —S—, —C(O)—, (hetero)cycle(s), and combinations thereof, in particular —O—C(O)—, —C(O)—O— or -(hetero)cycle-O-(hetero)cycle-, and preferably not being interrupted; X is as defined previously and preferably X denotes a sulfur atom; Y, AK1, AK3, u, v, R, R₄, Rₐ and R_{b} being as defined previously,
   it being understood that these compounds contain at least two radicals —C(O)—C(Rₐ)(R_{b})—C(O)—R₄.

According to a preferred embodiment, t is equal to 0 and u is equal to 1.

According to a preferred embodiment, all the radicals AK2 are identical. Preferably, all the radicals Y are identical.

According to a preferred embodiment, Y denotes —O—C(O)—.

According to this embodiment variant, the compounds of formula (Id), and also the optical or geometrical isomers thereof and/or the solvates thereof, such as hydrates, are in particular of formula (Iid) as defined previously.

According to a preferred embodiment, the treatment process according to the invention comprises the application to keratin materials, notably the skin and/or the lips and/or the eyelashes and/or the eyebrows, of at least one compound of formula (I) and also the optical and geometrical isomers thereof, and/or the solvates thereof, such as the hydrates, or a composition containing same, chosen from the following compounds of formula (Ie), and also the optical or geometrical isomers thereof and/or the solvates thereof, such as the hydrates:

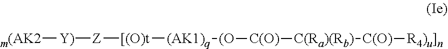

in which formula (Ie):
n+m is equal to 2, m being non-zero, and m preferably being equal to 2; n is as defined previously for the compounds of formula (I); preferably, n is zero;
Z represents a linear or branched, saturated or unsaturated, conjugated or non-conjugated, preferably non-conjugated, acyclic or cyclic, aromatic or non-aromatic, preferably non-aromatic, C₂ to C₂₄, in particular C₃ to C₁₂, divalent hydrocarbon-based radical, said radical Z also being interrupted with one or more heteroatoms or groups chosen from —O—, —N(Rₐ)—, —S—, —C(O)—, (hetero)cycle(s), and combinations thereof; preferably, Z denotes a divalent radical —(CH₂)_{g}-(heterocycle)-O—(CH₂)ₕ-(heterocycle)-(CH₂)ⱼ— in which h denotes 0, 1 or 2, preferably 0 or 1, g and j independently denote an integer between 1 and 3, preferably 1; (heterocycle) denotes a divalent heterocyclic radical (i.e. a heterocycle), preferably a heterocycloalkyl radical, more particularly a divalent 5- or 6-membered heterocycloalkyl radical, in particular a divalent tetrahydrofuran radical or a divalent tetrahydropyran radical, said heterocycle being optionally substituted with one or more radicals chosen from —OR with R as defined previously, and notably —OH or —O—C(O)—C(Rₐ)(R_{b})—C(O)—R₄;
AK2 represents a linear or branched, saturated or unsaturated, preferably saturated, C₁₄-C₃₀, preferably C₁₄-C₂₄, preferably C₁₇, monovalent hydrocarbon-based chain, said chain being optionally substituted with one or more identical or different radicals chosen from —OR and —X-AK3-(O—C(O)—C(Rₐ)(R_{b})—C(O)—R₄)ᵥ, preferably unsubstituted; and/or said chain being optionally interrupted with one or more heteroatoms or groups chosen from —O—, —S—, —C(O)—, (hetero)cycles, and combinations thereof, in particular —O—C(O)—, —C(O)—O— or -(hetero)cycle-O-(hetero)cycle-, preferably optionally interrupted with —O—C(O)— or —C(O)—O—, and more preferentially not being interrupted;
X is as defined previously; preferably, X denotes a sulfur atom;
Y, AK1, AK3, v, t, q, u, R, R₄, Rₐ and R_{b} being as defined previously;
it being understood that said compound contains at least two radicals —C(O)—C(Rₐ)(R_{b})—C(O)—R₄.

According to this embodiment variant, the compounds of formula (Ie), and also the optical or geometrical isomers thereof and/or the solvates thereof, such as hydrates, are in particular of formula (Iie) as defined previously.

Another subject of the invention is the compounds of formula (I) as described previously, and also the salts, isomers and/or solvates thereof, such as hydrates, different from compounds (1) to (7) as described previously.

Another subject of the invention is the compounds of formulae (Ia), (Ib), (Ic), (Id), and (Ie) as described previously, and also the salts, isomers and/or solvates thereof such as the hydrates, more particularly the compounds of formulae (Iic), (Iid), and (lie) as described previously.

In particular, another subject of the invention is compounds (8), (9), (10), (11), (12), (13), and (14) as described previously, and also the salts, isomers and solvates thereof.

### Preparation of the Compounds of Formula (I)

In particular, the compound(s) of formula (I), or (Ia), (Ib), (Ic), (Id), (Ie), (Iic), (Iid) or (Iie), may be prepared via the following sequence of reactions:

### First Step

In a first step, a commercial polyol, which is preferably natural, of formula (H)ₚ—Z(OH)_{m′}, is reacted with m equivalents of fatty derivatives R′CO-AK′₂ with m′ greater than or equal to m, and m being as defined previously, under conditions known to those skilled in the art, to form an ester bond Y according to the scheme below:

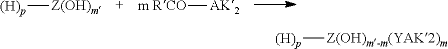

in which scheme:
Y represents —O—C(O)—;
R′ denoting in particular —OH or —Cl; preferably, R′ denotes —OH;
AK′2 represents a linear or branched, unsaturated, C₁-C₄₀, preferably C₁₄-C₂₀, hydrocarbon-based chain, said chain being optionally interrupted with one or more heteroatoms or groups chosen from —O—, —S—, —C(O)—, (hetero)cycle(s), and combinations thereof, in particular —O—C(O)—, —C(O)—O— or -(hetero)cycle-O-(hetero)cycle-, preferably interrupted with —O— or —S—, more preferentially not being interrupted.

According to a preferred embodiment, m is equal to m′.

The polyols (H)ₚ—Z(OH)_{m′} may be cyclic or acyclic.

By way of example of cyclic polyols (H)ₚ—Z(OH)_{m′}, mention may be made of maltose, lactose, raffinose, sucrose, trehalose, melibiulose, melibiose, mannobiose, kojibiose, nigerose, isomaltose, rutinose, rutinulose, xylobiose or sorbitan.

As examples of acyclic polyols (H)ₚ—Z(OH)_{m′}, mention may be made of ethylene glycol, propylene glycol, butylene glycol, pentylene glycol, hexylene glycol, butanediol, glycerol, pentaerythritol, dipentaerythritol, trimethylolethane, mannitol or trimethylolpropane.

According to a preferred embodiment, the polyol (H)ₚ—Z(OH)_{m′} is chosen from glycerol, mannitol, sucrose, ethylene glycol, sorbitan or trimethylolpropane, and more preferentially the polyol (H)ₚ—Z(OH)_{m′} denotes glycerol.

The compounds R′CO-AK′₂ are preferably unsaturated fatty acids containing from 15 to 36 carbon atoms, preferably from 15 to 24 carbon atoms. They may be mono- or polyunsaturated.

The monounsaturated fatty acids are in particular chosen from palmitoleic acid, oleic acid, 9-eicosenoic acid, 11-eicosenoic acid, erucic acid and nervonic acid.

The polyunsaturated fatty acids are notably chosen from linoleic acid, α-linolenic acid, γ-linolenic acid, dihomo-γ-linolenic acid, arachidonic acid, eicosapentaenoic acid, docosahexaenoic acid, ruminic acid and hydroxyl monounsaturated fatty acids notably chosen from 10-hydroxy-cis-12-octadecenoic acid, 10-hydroxy-trans-11-octadecenoic acid, 12-hydroxy-cis-9-octadecenoic acid, 13-hydroxy-cis-9-octadecenoic acid, ricinoleic acid, isoricinoleic acid, and polyunsaturated hydroxyl fatty acids notably chosen from densipolic acid, auricolic acid, dimorphecolic acid.

More preferentially, the unsaturated fatty acid is chosen from ricinoleic acid, oleic acid, palmitoleic acid, erucic acid and mixtures thereof.

### Second Step

In a second step, all or some of the unsaturation(s) of the radical(s) AK′2 are converted a) via an epoxidation reaction into epoxide to yield the derivatives (III), such as the starting compound a1 illustrated in the scheme described below, or b) via a thiol-ene reaction with a (poly)hydroxylated thiol or a thiol bearing acid function(s) to yield the derivatives (IV), for instance compound b3 illustrated in the scheme described below.

The thiol-ene reaction b) may be performed using (poly)hydroxylated thiols such as linear hydroxylated thiols of formula HS—(CH₂)ₖ—OH with k being an integer from 1 to 12, preferably from 1 to 6, such as 2-mercaptoethanol, 3-mercapto-1-propanol, 6-mercapto-1-hexanol, 4-mercapto-1-butanol, or such as branched hydroxylated thiols preferably including from 1 to 6 carbon atoms, such as 3-mercapto-3-methylbutanol, 3-mercapto-2-butanol, or such as polyhydroxylated thiols, preferably including from 1 to 6 carbon atoms, such as 3-mercapto-1,2-propanediol HS—CH(OH)—CH₂OH.

The compounds (IV) obtained are thus substituted with a radical —S-AK3-(OH)ᵥ with v being equal to 1 if the thiol is hydroxylated and v being greater than 1, such as 2, if the thiol is polyhydroxylated, AK3 and v being as defined previously for the compounds of formula (I).

### Third Step

In a third step, all or some of the epoxide groups of the compounds of formula (III) are opened with nucleophilic compounds, in particular i) alcohols preferably of C₁-C₄ such as methanol or ethanol, or even a polyol to give compounds (IIIi), such as compound b2 illustrated in the scheme hereinbelow, ii) carboxylic acids, preferably of C₁-C₆, such as lactic acid, pyruvic acid, benzoic acid, glycolic acid or 2,2-bishydroxymethylpropionic acid, more preferentially carboxylic acids substituted with at least one hydroxyl radical such as lactic acid, to yield the compounds (IIIii), such as compound b1 illustrated in the scheme hereinbelow.

According to a preferred embodiment, the carboxylic acid ii) is chosen from lactic acid, glycolic acid or 2,2-bishydroxymethylpropionic acid (epoxy-acid reaction) and the reaction is performed in the presence of a catalyst such as imidazole, 1-methylimidazole, N,N-dimethylbenzylamine or caffeine.

According to a preferred embodiment, the alcohol i) is chosen from ethylene glycol, propylene glycol, butylene glycol, pentylene glycol, hexylene glycol, butanediol, glycerol, pentaerythritol, dipentaerythritol, trimethylolethane and trimethylolpropane, more preferentially butylene glycol or trimethylolethane.

### Fourth Step

In a fourth step, all or some of the alcohol functions-OH of the compounds (IIIi) or (IIIii) and all or some of the alcohol functions which may possibly still be present originating from the polyols (H)ₚ—Z(OH)_{m′} used in the first step are converted into radicals —O—C(O)—C(Rₐ)(R_{b})—C(O)—R₄, for example via a (trans) esterification reaction with an ester Gp-C(O)—C(Rₐ)(R_{b})—C(O)—R₄, in which Gp preferably denotes a hydroxyl radical or a (C₁-C₄)alkoxy radical, such as methoxy, ethoxy, isobutoxy or t-butoxy.

According to a preferred embodiment, Gp-C(O)—C(Rₐ)(R_{b})—C(O)—R₄ denotes an ester chosen from ethyl acetoacetate, ethyl 2-methylacetoacetate and tert-butyl acetoacetate, preferably tert-butyl acetoacetate.

The compounds of structures (Ia), (Ic), such as the compounds of structure (Iic), and the compounds of structures (Id) and (Ie) may be obtained according to this process.

By way of example, step 4 may be performed according to the protocol described in Trevino, A. S., and Trumbo, D. L. (2002). Acetoacetylated castor oil in coating applications. Progress in Organic Coatings, 44 (1), 49-54:

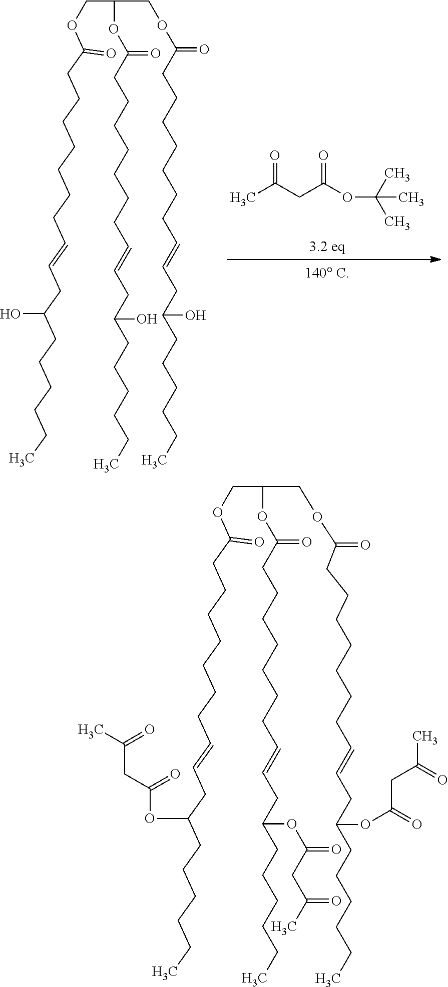 [SMILES: CCCCCCC(O)C/C=C/CCCCCCCC(=O)OCC(COC(=O)CCCCCCC/C=C/CC(O)CCCCCC)OC(=O)CCCCCCC/C=C/CC(O)CCCCCC ; CC(=O)CC(=O)OC(C)(C)C ; CCCCCCC(C/C=C/CCCCCCCC(=O)OCC(COC(=O)CCCCCCC/C=C/CC(CCCCCC)OC(=O)CC(C)=O)OC(=O)CCCCCCC/C=C/CC(CCCCCC)OC(=O)CC(C)=O)OC(=O)CC(C)=O]

In certain cases, the compounds (H)ₚ—Z(OH)_{m′-m} (YAK′2) m resulting from the first step are commercial; in particular, certain compounds for which m′ is equal to m, p is equal to 0, Z preferably denoting —CH₂—CH—CH₂—, are commercial. When p is equal to 0 and m′ is equal to m, the compounds are esters of optionally hydroxylated unsaturated fatty acids, in particular triglycerides of castor oil, soybean oil, canola oil or linseed oil. The synthesis of compounds (Ia) to (Ie) then begins from the second step described above.

For example, starting with an unsaturated triglyceride, a thiol-ene reaction is performed with a (poly)hydroxyl thiol such as SH—(CH₂)ₓOH with x being an integer preferably from 1 to 4, and then a transesterification reaction (as described in Desroches, M., Caillol, S., Lapinte, V., Auvergne, R. and Boutevin, B. (2011). Synthesis of biobased polyols by thiol-ene coupling from vegetable oils. Macromolecules, 44 (8), 2489-2500:

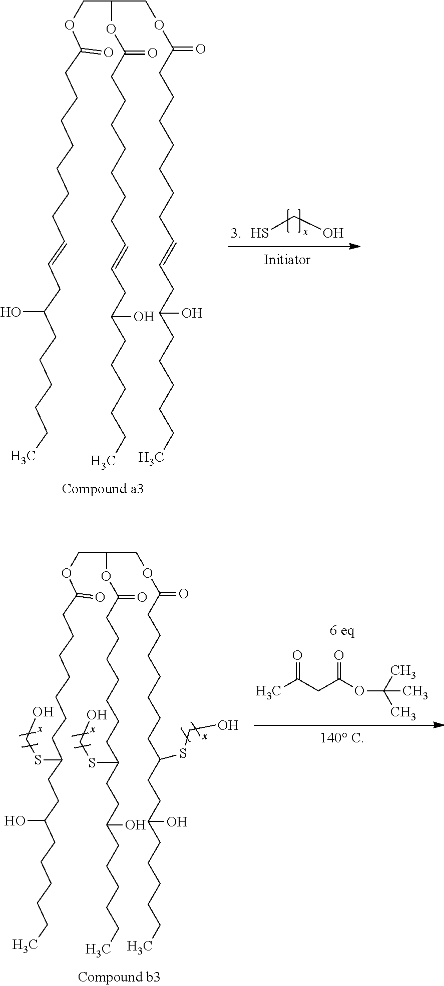

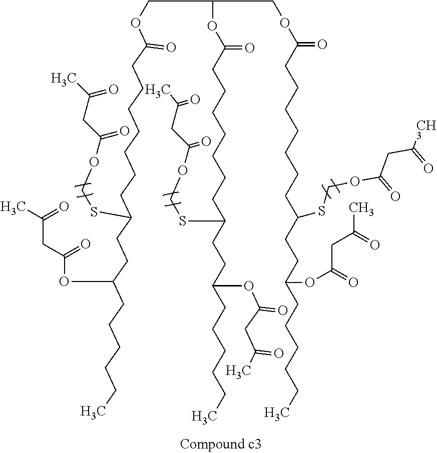

For example, starting with an unsaturated triglyceride, a total or partial epoxidation reaction is performed, followed by opening of the epoxide(s) formed, which generates alcohol functions and (trans) esterification. A commercial epoxidized triglyceride, such as the products sold by Arkema under the trade names Vikoflex 7170 or Vikoflex 7190, may also be used as starting material.

By way of example, the reaction scheme below may be used, in which all the epoxides are opened with lactic acid and each hydroxyl is then esterified with t-Bu-OCOCH₂CH₃, according to the scheme below:

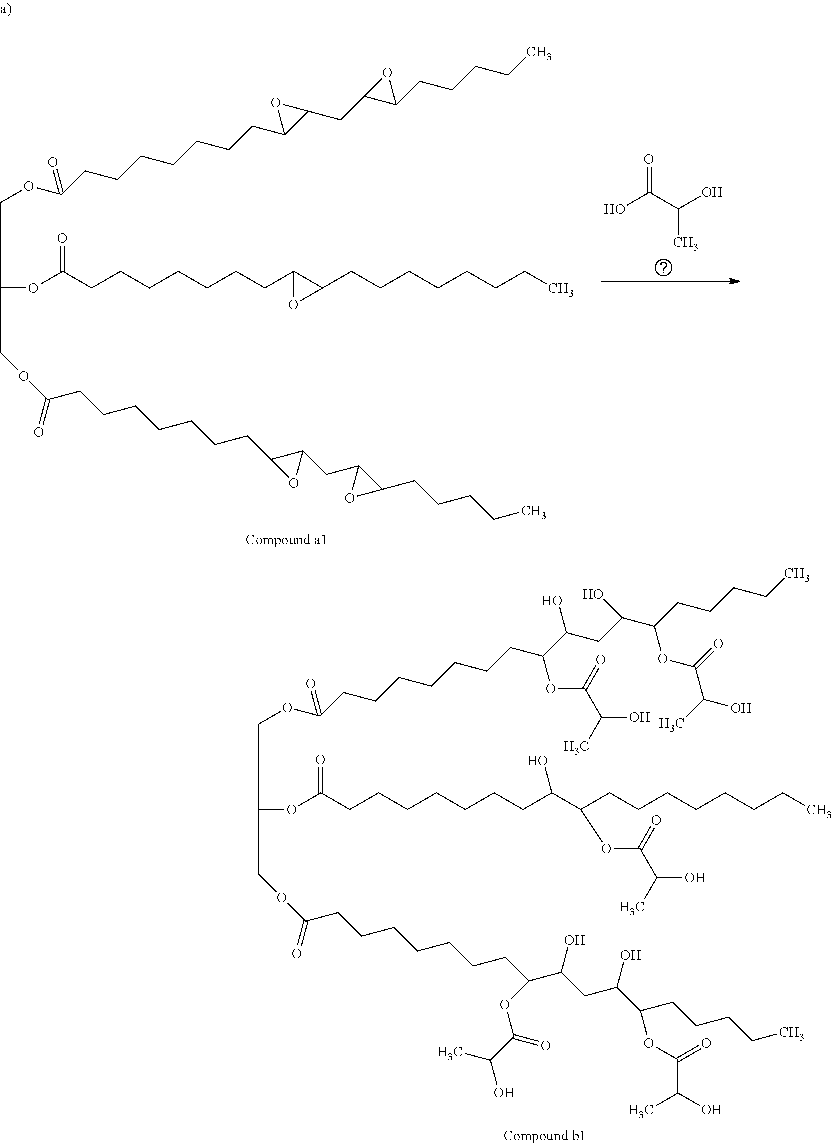

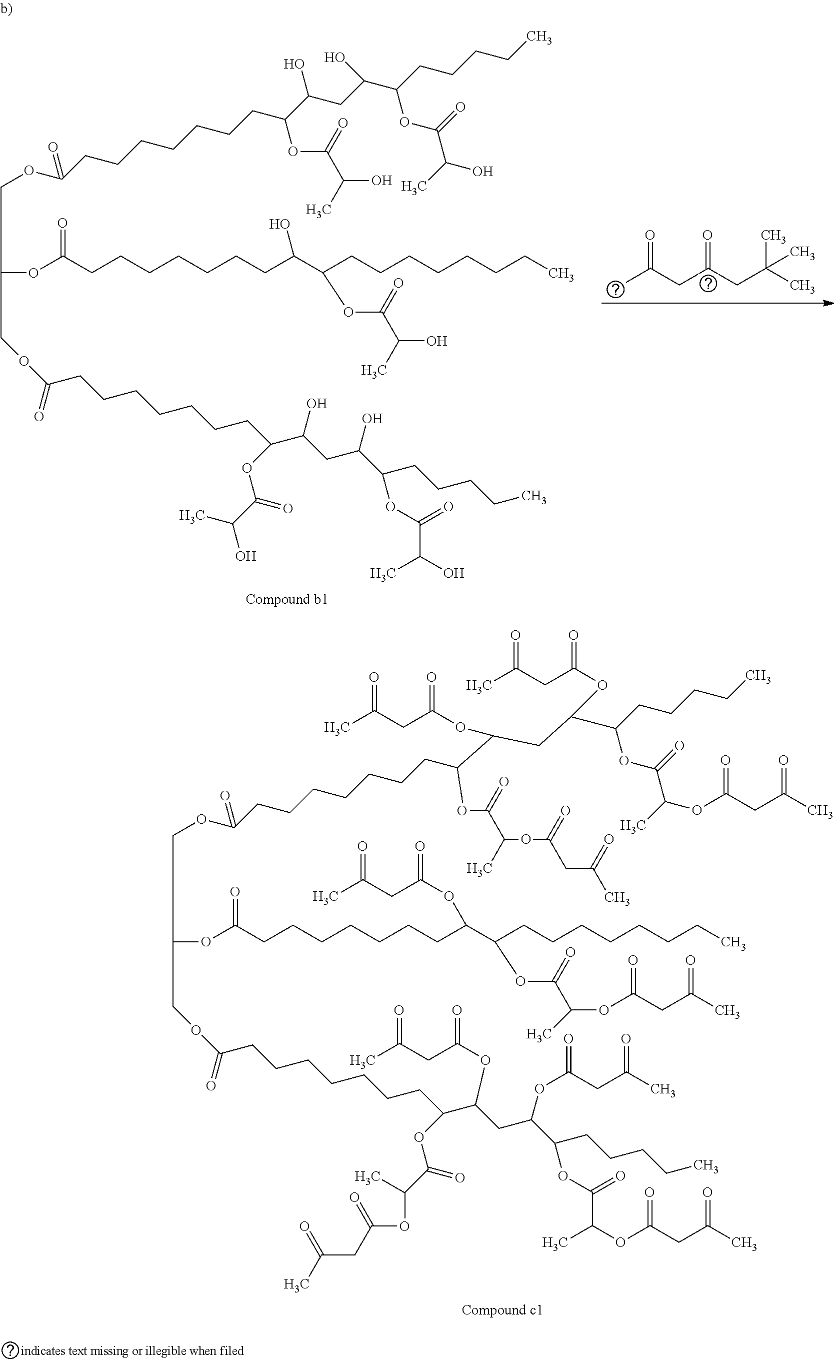 [SMILES: CCCCCCCCC(OC(=O)C(C)O)C(O)CCCCCCCC(=O)OC(COC(=O)CCCCCCCC(OC(=O)C(C)O)C(O)CC(O)C(CCCCC)OC(=O)C(C)O)COC(=O)CCCCCCCC(OC(=O)C(C)O)C(O)CC(O)C(CCCCC)OC(=O)C(C)O ; CC(=O)CC(=O)CC(C)(C)C ; CCCCCCCCC(OC(=O)C(C)OC(=O)CC(C)=O)C(CCCCCCCC(=O)OC(COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)OC(=O)CC(C)=O]

By way of example, the reaction scheme below may also be used, in which all the epoxides are opened with an alcohol, methanol in the example, and each hydroxyl is then esterified with t-Bu-OCOCH₂CH₃:

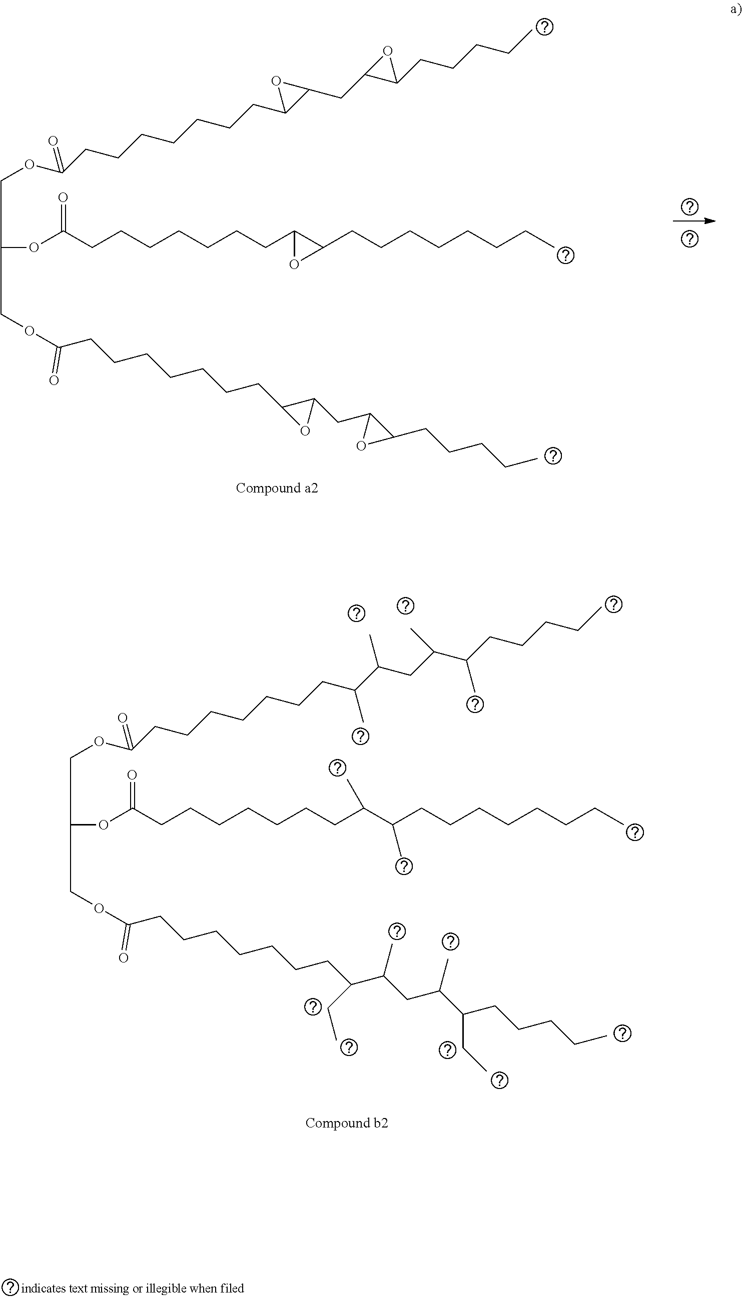 [SMILES: CCCCCCCCC1OC1CCCCCCCC(=O)OC(CCOC(=O)CCCCCCCC1OC1CC1OC1CCCCC)CCOC(=O)CCCCCCCC1OC1CC1OC1CCCCC ; CCCCCCCCC(C)C(C)CCCCCCCC(=O)OC(CCOC(=O)CCCCCCCC(C)C(C)CC(C)C(C)CCCCC)CCOC(=O)CCCCCCCC(CC)C(C)CC(C)C(CC)CCCCC]

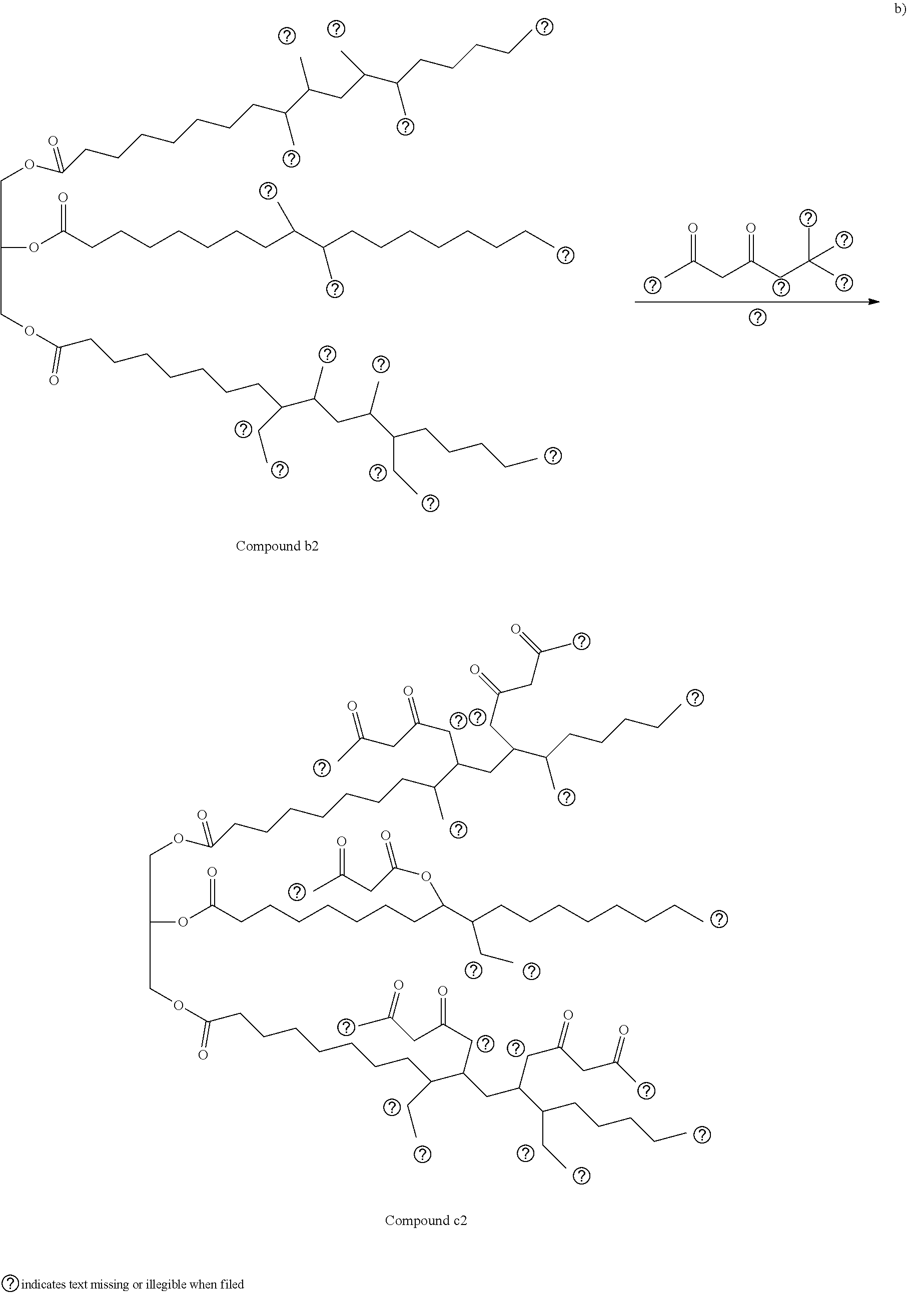 [SMILES: CCCCCCCCC(C)C(C)CCCCCCCC(=O)OC(CCOC(=O)CCCCCCCC(C)C(C)CC(C)C(C)CCCCC)CCOC(=O)CCCCCCCC(CC)C(C)CC(C)C(CC)CCCCC ; CC(=O)CC(=O)CC(C)(C)C ; CCCCCCCCC(CC)C(CCCCCCCC(=O)OC(CCOC(=O)CCCCCCCC(C)C(CC(=O)CC(C)=O)CC(CC(=O)CC(C)=O)C(C)CCCCC)CCOC(=O)CCCCCCCC(CC)C(CC(=O)CC(C)=O)CC(CC(=O)CC(C)=O)C(CC)CCCCC)OC(=O)CC(C)=O]

The compounds of formulae (Ia) to (Ie) may also be prepared via the following sequence of reactions:
In a first step, a preferably natural commercial polyacid (H)ₚ—Z(COOH)_{m′} is reacted with m equivalents of fatty alcohol HO-AK′2 with m′ greater than or equal to m, and m being as defined previously, under conditions known to those skilled in the art to form an ester bond Y according to the scheme below:

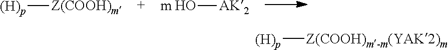

in which Y denotes —C(O)—O—, and AK′2 represents a monovalent linear or branched, unsaturated, C₁-C₄₀, preferably C₁₄-C₂₀, hydrocarbon-based chain, said chain optionally being interrupted with one or more heteroatoms or groups chosen from —O—, —S—, —C(O)—, (hetero)cycle(s), and combinations thereof, in particular —O—C(O)—, —C(O)—O—, -(hetero)cycle-O-(hetero)cycle-, preferably interrupted with —O— or —S—, and more preferentially not being interrupted.

According to a preferred embodiment, m is equal to m′.

The polyacids (H)ₚ—Z(COOH)_{m′} may be cyclic or acyclic.

The polyacids may preferably denote optionally hydroxylated dicarboxylic acids, optionally hydroxylated tricarboxylic acids, or optionally hydroxylated tetracarboxylic acids.

The non-hydroxylated dicarboxylic acids are notably chosen from maleic acid, propanedioic acid, butanedioic acid, pentanedioic acid, hexanedioic acid, decanedioic acid, dodecanedioic acid, cyclopropanedicarboxylic acid, cyclohexanedicarboxylic acid, cyclobutanedicarboxylic acid, naphthalene-1,4-dicarboxylic acid, naphthalene-2,3-dicarboxylic acid, naphthalene-2,6-dicarboxylic acid, suberic acid, oxalic acid, malonic acid, succinic acid, phthalic acid, terephthalic acid, isophthalic acid, tetrahydrophthalic acid, hexahydrophthalic acid, pimelic acid, sebacic acid, azelaic acid, glutaric acid, adipic acid, fumaric acid and itaconic acid, and preferably propanedioic acid, butanedioic acid and pentanedioic acid.

The hydroxylated dicarboxylic acids are notably chosen from tartaric acid, malic acid, aleuritic acid, 7,18-dihydroxytetracos-15-enoic acid and avenic acid B, and preferably malic acid and tartaric acid.

The (hydroxy)tricarboxylic acids are notably chosen from 2-oxaloglutaric acid, oxalosuccinic acid, 3-carboxymethylmuconic acid, 2-methylcitric acid, citric acid, isocitric acid, aconitic acid, transaconitic acid, 1,2,3-propanetricarboxylic acid, 1,2,5-pentanetricarboxylic acid, 1,3,5-benzenetricarboxylic acid, 1,3,5-cyclohexanetricarboxylic acid, 3-butene-1,2,3-tricarboxylic acid, 3-butene-1,1,3-tricarboxylic acid, trimellitic acid, 1,2,3-benzenetricarboxylic acid, 1,3,5-benzenetricarboxylic acid and agaric acid, preferably citric acid and aconitic acid.

The tetracarboxylic acids are notably chosen from 1,2,3,4-butanetetracarboxylic acid, pyromellitic acid, oxydisuccinic acid, thiodisuccinic acid, N-[1,2-dicarboxyethyl]-L-aspartic acid, ethylenediaminetetraacetic acid, ethylenediaminetetrapropionic acid and N,N′-ethylenebis(L-aspartic acid).

According to a preferred embodiment, the fatty alcohols HO-AK′2 are chosen from fatty alcohols comprising from 15 to 36 carbon atoms, preferably from 15 to 24 carbon atoms.

The fatty alcohols HO-AK′2 may be mono- or polyunsaturated (presence of one or more ethylenic double bonds).

The fatty alcohols HO-AK′2 may contain several alcohol functions.

The monounsaturated fatty alcohols are notably chosen from oleyl alcohol, docosenol, or unsaturated fatty alcohol ethers, such as polyoxyethylene monooleyl ether, preferably oleyl alcohol. The unsaturated monounsaturated fatty alcohols containing several alcohol functions are notably chosen from 9-octadecene-1,12-diol.

The polyunsaturated fatty alcohols are notably chosen from linoleic alcohol, 2,4-dodecadien-1-ol and dolichols (with n being greater than or equal to 3 and less than or equal to 5).

According to a preferred embodiment, the fatty alcohol HO-AK′2 is chosen from oleyl alcohol, linoleyl alcohol or mixtures thereof.

The following steps are identical to those described previously:
In a second step, all or some of the unsaturation(s) of the radical(s) AK′2 are converted a) via an epoxidation reaction into epoxide to yield derivatives (III′), such as compound a4 illustrated in the scheme below, or b) via a thiol-ene reaction with a (poly)hydroxylated thiol or a thiol bearing acid function(s) to yield derivatives (IV′), such as compound b5 illustrated in the scheme below.
In a third step, all or some of the epoxide groups of the compounds of formula (III′) are opened with nucleophilic compounds, such as i) preferably C₁-C₄ alcohols such as methanol or ethanol or even a polyol to yield compounds (III′i), such as compound b4 illustrated in the scheme below, ii) carboxylic acids preferably of C₁-C₆, such as lactic acid, pyruvic acid or benzoic acid, more preferentially carboxylic acids substituted with at least one hydroxyl radical, such as lactic acid, to yield compounds (III′ii), such as compound b6 illustrated in the scheme below.
In a fourth step, all or some of the alcohol functions-OH of the compounds (III′i) or (III′ii) and all or some of the alcohol functions which may still be present and which are derived from the polyols (H)ₚ—Z(OH)_{m′} used in the first step are converted into radicals —O—C(O)—C(Rₐ)(R_{b})—C(O)—R₄, for example via a transesterification reaction with an ester Gp-C(O)—C(Rₐ)(R_{b})—C(O)—R₄ in which Gp preferably denotes a hindered alkoxy radical such as tBu-O—, to give the corresponding compounds of formula (I).

The compounds of formulae (Ia) to (Ie) such that p is equal to 1, Y denotes —C(O)—O— and m is equal to 1 may also be prepared by combining steps 2, 3 and 4 described previously, from an unsaturated fatty acid such as oleic acid as described in “The synthesis of Bio-based Michael donors from tall oil fatty acids for polymer development”, Polymers, 2022, 14, 4107.

By way of example, the reaction scheme below may be used, in which all the epoxides are opened with methanol and each hydroxyl is then esterified with t-Bu-OCOCH₂CH₃:

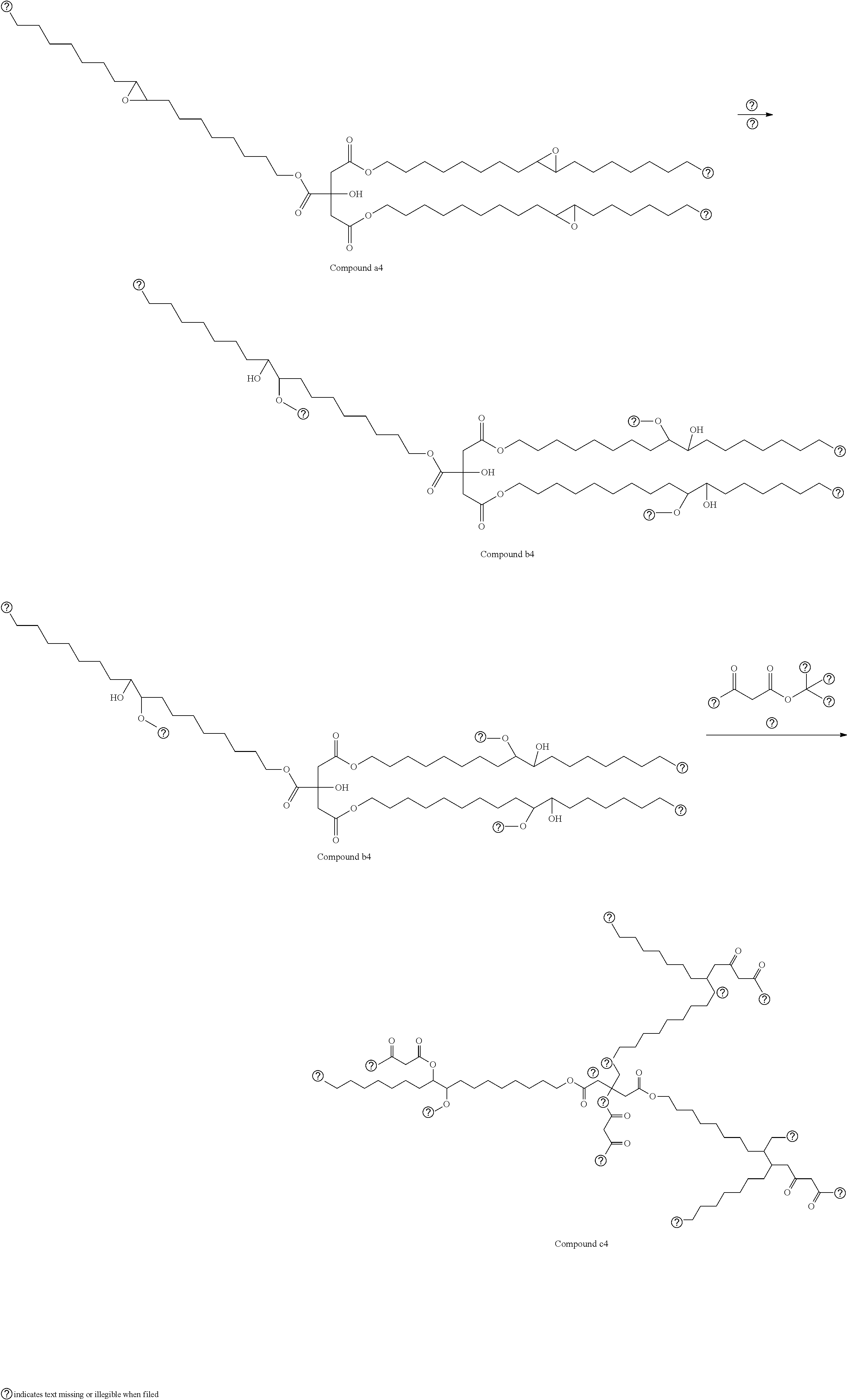

By way of example, the reaction scheme below may be used, in which an unsaturated oil reacts with a (poly)hydroxyl thiol such as SH—(CH₂)ₓOH, with x being an integer preferably from 1 to 4, followed by a transesterification reaction (as described in Desroches, M., Caillol, S., Lapinte, V., Auvergne, R. and Boutevin, B. (2011). Synthesis of biobased polyols by thiol-ene coupling from vegetable oils. Macromolecules, 44 (8), 2489-2500):

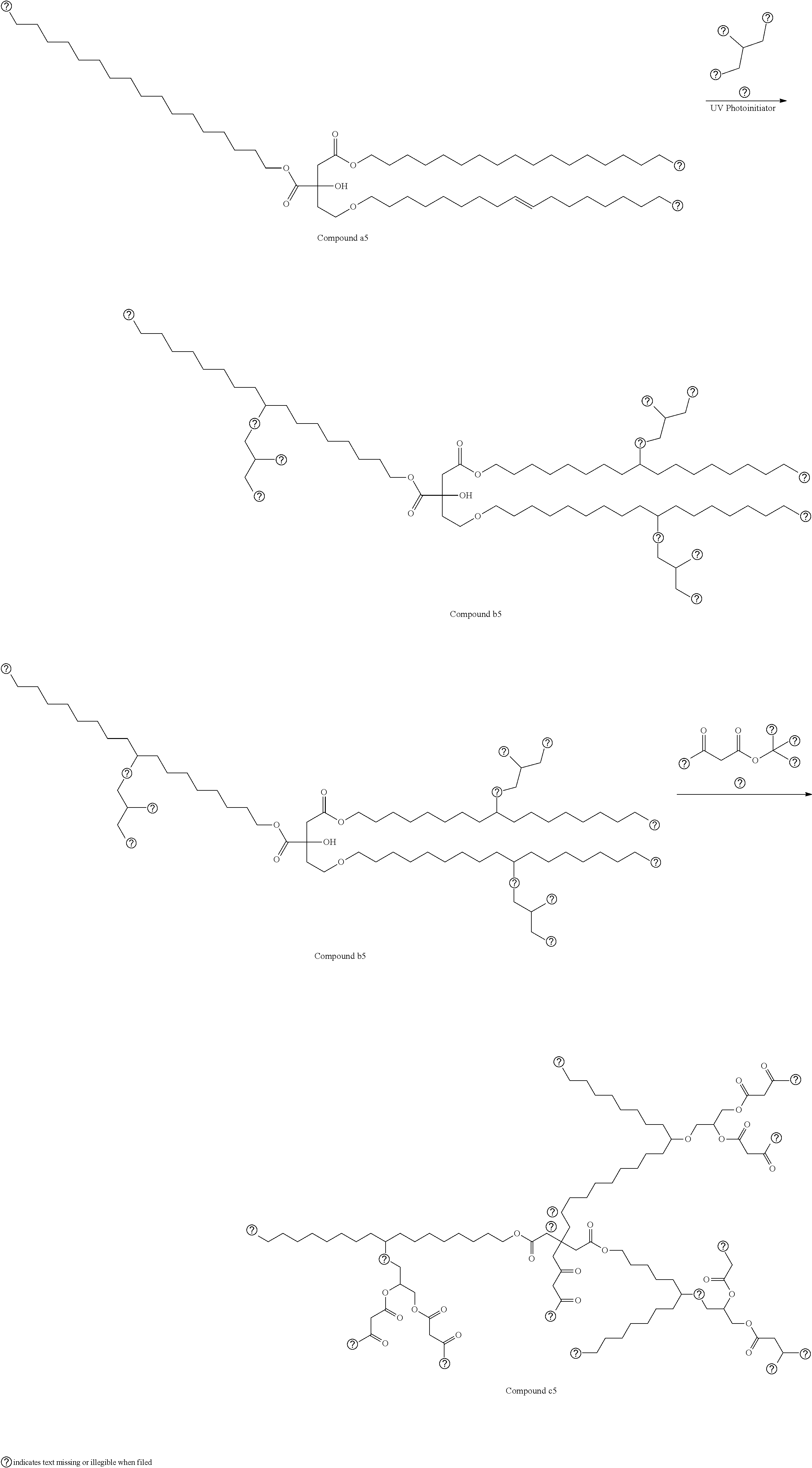

By way of example, the reaction scheme below may be used, in which all the epoxides are opened with lactic acid and each hydroxyl is then esterified with t-Bu-OCOCH₂CH₃:

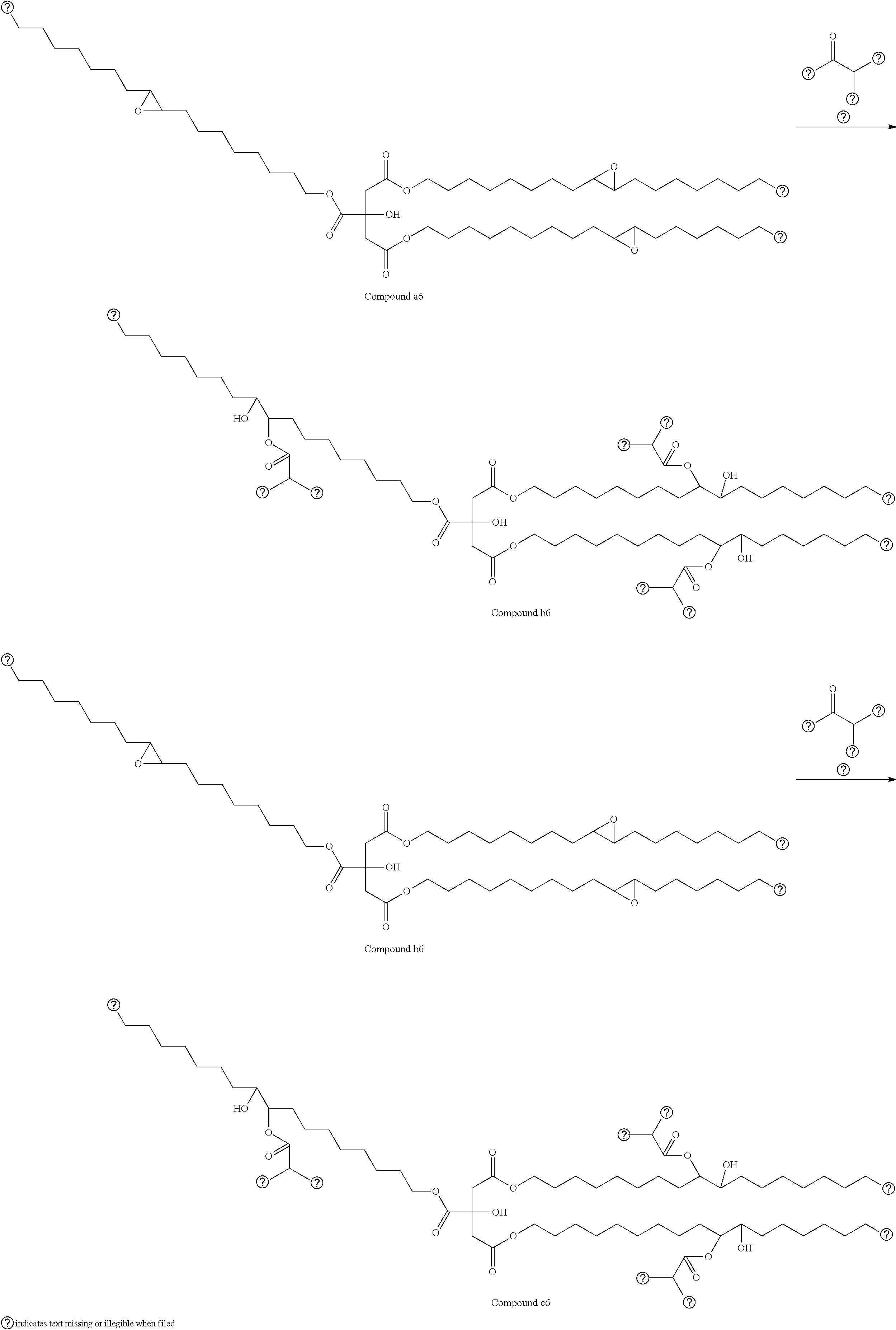

### Crosslinking Agent ii)

The treatment process according to the invention as described previously comprises the application to keratin materials, notably the skin and/or the lips and/or the eyelashes and/or the eybrows, of (i) at least one or more compounds of formula (I) as defined previously, and (ii) at least one crosslinking agent.

For the purposes of the invention, the term “crosslinking agent”, also termed “R”, denotes a compound that is capable of establishing with at least one acetoacetate function of the compound(s) of formula (I) used in the treatment process according to the invention at least one covalent bond, and thus of crosslinking this or these compounds.

Preferably, the term “crosslinking agent”, also termed “R”, refers to a compound that is capable of establishing at least one covalent bond with an acetoacetate function of the compound(s) of formula (I) used in the treatment process according to the invention and thus of crosslinking this or these compounds.

For the purposes of the present invention, it is understood that the terms “crosslinking agent” and “crosslinker” are equivalent.

Compositions “C2”, “C3” and “C4” as defined previously contain at least ii) a crosslinking agent. The compositions of the invention may comprise a fatty phase, an aqueous phase or may be in the form of a direct or inverse emulsion. Composition “C4” may be an aqueous composition.

According to one aspect, the treatment process of the invention uses a composition termed “C3”, in particular a cosmetic composition for keratin materials, notably the skin and/or the lips and/or the eyelashes and/or the eyebrows, in particular for caring for and/or making up keratin materials, notably the skin, the lips, the eyelashes and/or eyebrows, comprising i) at least one compound of formula (I), as defined previously and hereinbelow, ii) at least one crosslinking agent, in particular as defined previously and hereinbelow, and iii) at least one cosmetic active agent, in particular as defined previously and hereinbelow.

The crosslinking agent(s) ii) are preferably present in a mass content ranging from 0.2% to 60% by weight, in particular ranging from 0.5% to 40% by weight, more particularly from 1% to 35% by weight, even more particularly from 1% to 30% by weight, relative to the total weight of the composition containing same.

In particular, in composition “C2” or “C3”, the crosslinking agent(s) ii) and the compound(s) i) as defined previously are preferably present in a mass content ranging from 1% to 35% by weight, relative to the total weight of the composition comprising them.

When the treatment process involves several steps including a step of using one or more compounds of formula (I) or (Ia), (Ib), (Ic), (Id), (Ie), (Iic), (Iid) and/or (Iie) without crosslinking agent, the compound(s) of formula (I), (Ia), (Ib), (Ic), (Id), (Ie), (Iic), (Iid) or (Iie) may be used on their own (pure), or in a composition “C1” as described previously.

More precisely, the crosslinking agent(s) ii) that are suitable for use in the invention may be chosen from compounds bearing amine, thiol, acrylate and/or carbonyl functions, such as a ketone or aldehyde function. A crosslinking agent R may also denote a metal alkoxide.

Thus, according to a particular embodiment, the crosslinking agent ii) is chosen from (poly)amine, (poly)thiol, (poly)carbonyl, (poly)acrylate and metal alkoxide compounds and mixtures thereof, and preferably chosen from (poly)amine compounds.

The term “(poly)amine, (poly)thiol, (poly)carbonyl and (poly)acrylate compounds” is intended to denote compounds including at least one primary or secondary amine, thiol, carbonyl (such as a ketone or aldehyde function) or acrylate function, respectively.

The metal alkoxide compounds are defined below.

### A) (Poly)Amine Compounds

According to a preferred embodiment, the crosslinking agent R is chosen from (poly)amine compounds.

The (poly)amine compound may be chosen in particular from polyamine compounds bearing several primary and/or secondary amine groups or from amino alkoxysilanes, and more particularly from amino alkoxysilane compounds, diamine compounds, triamine compounds, and mixtures thereof.

The (poly)amine compound may be a compound comprising from 2 to 20 carbon atoms, notably a nonpolymeric compound; they may be acyclic or cyclic, linear or branched, saturated or unsaturated, conjugated or non-conjugated, aromatic or non-aromatic, optionally interrupted with one or more heteroatoms or groups chosen from —O—, —S—, —Si(R′)₂—, —N(R″)—, preferably —O—, —Si(R′)₂—, or combinations thereof such as —Si(R′)₂—O— or —O—Si(R′)₂—, with R′, which may be identical or different, representing a (C₁-C₄)alkyl group such as methyl, and R″ representing a hydrogen atom or a (C₁-C₄)alkyl group, preferably a hydrogen atom.

The term “non-polymeric compound” means a compound which is not directly obtained via a monomer polymerization reaction.

(Poly)amine compounds that may be mentioned in particular include N-methyl-1,3-diaminopropane, N-propyl-1,3-diaminopropane, N-isopropyl-1,3-diaminopropane, N-cyclohexyl-1,3-diaminopropane, 2-(3-aminopropylamino) ethanol, 3-(2-aminoethyl)aminopropylamine, bis(3-aminopropyl)amine, methylbis(3-aminopropyl)amine, N-(3-aminopropyl)-1,4-diaminobutane, N,N-dimethyldipropylenetriamine, 1,2-bis(3-aminopropylamino) ethane, N,N′-bis(3-aminopropyl)-1,3-propanediamine, ethylenediamine, 1,3-propylenediamine, 1,4-butylenediamine, lysine, cystamine, xylenediamine, tris(2-aminoethyl)amine, 1,3-bis(aminomethyl)cyclohexane, 1,4-bis(aminomethyl)cyclohexane, diaminopropanol, 4,7,10-trioxa-1,13-tridecanediamine, spermidine and C36-alkylenediamines (Priamine™ 1071, 1073, 1074, 1075, respectively), preferably 4,7,10-trioxa-1,13-tridecanediamine.

According to a particular embodiment of the invention, the (poly)amine compounds are monoamine compounds, i.e. they contain only one primary and/or secondary amine group, preferably a primary amine group (NH₂).

The (poly)amine compound(s) may be chosen from amino alkoxysilanes, notably of formula R′₁Si(OR′₂)_{z}(R′₃)ₓ in which:
R′₁ is a linear or branched, saturated or unsaturated, cyclic or acyclic C₁-C₆ hydrocarbon-based chain substituted with a group chosen from primary amine groups NH₂ or secondary amine groups —N(H)R with R representing a C₁-C₄ alkyl, an aryl or a benzyl substituted with an amino group or with a C₁-C₄ aminoalkyl group; R′₁ may be interrupted in its chain with a heteroatom (O, S, NH) or a carbonyl group (CO), R′₁ being linked to the silicon atom directly via a carbon atom,
R′₂ and R′₃, which may be identical or different, represent a linear or branched (C₁-C₆)alkyl group,
z denotes an integer ranging from 1 to 3, and
x denotes an integer ranging from 0 to 2,
   with z+x=3.

In particular, R′₁ is an acyclic chain. Preferably, R′₁ is a linear or branched, saturated or unsaturated C₁-C₆ hydrocarbon-based chain substituted with an amine —NH₂ or —N(H)R group, with R representing a C₁-C₆ alkyl, a C₃-C₆ cycloalkyl or a C₆ aromatic group. More preferentially, R′₁ is a saturated linear C₁-C₆ hydrocarbon-based chain substituted with an amine group NH₂. Even more preferentially, R′₁ is a saturated linear C₂-C₄ hydrocarbon-based chain substituted with an amine group NH₂.

In particular, R′₂ represents an alkyl group comprising from 1 to 4 carbon atoms; preferably, R′₂ represents a linear alkyl group comprising from 1 to 4 carbon atoms and more preferentially R′₂ represents an ethyl group.

In particular, R′₃ represents an alkyl group comprising from 1 to 4 carbon atoms; preferably, R′₃ represents a linear alkyl group comprising from 1 to 4 carbon atoms and more preferentially R′₃ represents methyl or ethyl groups. Preferably, z is equal to 3.

In particular, the (poly)amine compound(s) are chosen from amino alkoxysilanes including only one primary and/or secondary, preferably primary (NH₂), amine group, such as 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane, and N-(2-aminoethylaminomethyl)phenethyltrimethoxysilane.

Preferably, the (poly)amine compound(s) are chosen from 3-aminopropyltriethoxysilane (APTES), 3-aminoethyltriethoxysilane (AETES), 3-aminopropylmethyldiethoxysilane, and N-(2-aminoethyl)-3-aminopropyltriethoxysilane, and more preferentially 3-aminopropyltriethoxysilane (APTES), in particular the product sold by Sigma Aldrich.

The (poly)amine compound may also be chosen from amino polymers, notably having a weight-average molecular weight ranging from 500 g·mol⁻¹ to 1 000 000 g·mol⁻¹, preferably ranging from 500 g·mol⁻¹ to 500 000 g·mol⁻¹, and preferentially ranging from 500 g·mol⁻¹ to 100 000 g·mol⁻¹.

According to a particular embodiment of the invention, the (poly)amine compounds are monoamine compounds and are chosen from polydialkylsiloxanes notably of formula (IV):

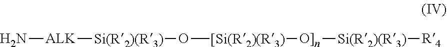

in which formula (IV):
ALK represents a (C₁-C₄)alkylene group which is linear or branched, preferably linear, such as propylene,
R′₂ and R′₃, which may be identical or different, preferably identical, represent a (C₁-C₄)alkyl group, such as methyl, and
R′₄ represents a linear or branched (C₁-C₆)alkyl group, preferably C₄, such as n-butyl, n represents an integer greater than or equal to 2, preferably the value of n is such that the weight-average molecular weight of the polydimethylsiloxane ranges from 500 to 3000 g·mol⁻¹. As examples of polydimethylsiloxanes (V), mention may be made of the products sold under the names MCR-A11 and MCR-A12 by the company Gelest.

According to a particular embodiment of the invention, the (poly)amine compounds are diamine compounds, i.e. they contain two primary and/or secondary amine groups, preferably primary amine groups (NH₂).

More particularly, they are chosen from the compounds of formula (V) or (VI):

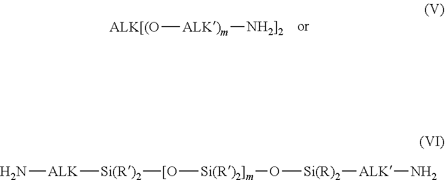

in which formulae (V) and (VI):
ALK and ALK′, which may be identical or different, represent a linear or branched (C₁-C₆)alkylene group, preferably a linear group such as propylene,
R′, which may be identical or different, represents a (C₁-C₄)alkyl group such as methyl,
m represents an integer greater than or equal to 0; preferably, the value of m is such that the weight-average molecular weight of compound (V) or (VI) ranges from 500 g·mol⁻¹ to 55 000 g·mol⁻¹.

As examples of compounds of formula (VI), mention may be made of those sold under the names DMS-A11, DMS-A12, DMS-A15, DMS-A21, DMS-A31, DMS-A32 and DMS-A35 by the company Gelest.

The (poly)amines compounds that are diamines are particularly polyether diamines notably of formula H₂N-ALK-O-[ALK′—O]ₘ-ALK″—NH₂ with ALK, ALK′ and ALK″, which may be identical or different, representing a linear or branched (C₁-C₆))alkylene group, and m representing an integer greater than or equal to 0, such as 4,7,10-trioxa-1,13-tridecanediamine or the compounds known under the reference Jeffamine from the company Hunstman, and more particularly α,ω-diamino polyethylene glycol and/or polypropylene glycol (with an amine function at the end of the chain) such as the products sold under the names Jeffamine D-230, D-400, D-2000, D-4000, ED-600, ED-9000 and ED-2003.

According to a particular embodiment of the invention, the (poly)amine compound(s) are triamine compounds, i.e. they contain three primary and/or secondary amine groups, preferably primary amine groups (NH₂). More particularly, they are chosen from polyether triamines notably of formula ALK′″[(O-ALK′)ₘ-NH₂]₃ with ALK′ as defined previously and ALK′″ representing a linear or branched trivalent (C₁-C₆)alkylene group, and m representing an integer greater than or equal to 0.

As (poly)amine compounds that are triamine compounds, mention may be made in particular of polyether triamines, and notably α,ω-diamino polyethylene glycol and/or polypropylene glycol (with an amine function at the end of the chain) such as the products sold under the names Jeffamine T-403.

According to another particular embodiment of the invention, the (poly)amine compound(s) include more than three primary and/or secondary amine groups, preferably primary amine groups (NH₂).

In this variant, the (poly)amine compound(s) are chosen from poly(meth)acrylates or poly(meth)acrylamides bearing lateral primary or secondary amine functions, such as poly(3-aminopropyl)methacrylamide and poly(2-aminoethyl)methacrylate.

Preferably, the (poly)amine compounds are chosen from chitosans (notably poly(D-glucosamine)) and polydimethylsiloxanes comprising primary amine groups at the end of the chain and/or on side chains.

According to this variant, the (poly)amine compound(s) are chosen in particular from poly((C₂-C₅)alkyleneimines), and preferably polyethylenimines and polypropyleneimines, notably poly(ethyleneimine), in particular the product sold under reference 408700 by the company Aldrich Chemical or under the trade name Lupasol by BASF, notably with a molecular weight of between 1200 and 25 000; poly(allylamine), in particular the product sold under reference 479136 by the company Aldrich Chemical; polyvinylamines and copolymers thereof, notably with vinylamides, in particular vinylamine/vinylformamide copolymers such as those sold under the name Lupamin® 9030 by the company BASF; polyamine acids containing NH₂ groups, such as polylysine, in particular the product sold by the company JNC Corporation (formerly Chisso); amino dextran, in particular the product sold by the company CarboMer Inc; amino polyvinyl alcohol, in particular the product sold by the company CarboMer Inc; acrylamido (C₁-C₆)alkylamine-based copolymers, notably acrylamidopropylamine-based copolymers; and poly(D-glucosamine), for example sold under the reference Kionutrime CSG® by the company Kytozyme.

According to a particular embodiment, the polydimethylsiloxanes comprising primary amine groups at the end of the chain and/or on side chains are chosen from the compounds of formula (VII) below:

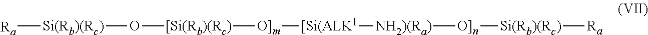

in which formula (VII):
Rₐ, which may be identical or different, represents a hydroxyl or (C₁-C₄)alkyl group,
R_{b} and R_{c}, which may be identical or different, preferably identical, represent a (C₁-C₄)alkyl group, such as methyl,
ALK¹ represents a linear or branched (C₁-C₆)alkylene group, optionally interrupted with an N(H) group,
m and n are integers greater than or equal to 1; preferably, m and n are such that the weight-average molecular mass of the compound of formula (VII) ranges from 1000 g·mol⁻¹ to 500 000 g·mol⁻¹.

According to a preferred variant, formula (VII) is such that Rₐ, R_{b} and R_{c} represent a methyl group, ALK¹ represents a propylene group, n and m are such that the weight-average molecular weight of the polydimethylsiloxane ranges from 1000 g·mol⁻¹ to 55 000 g·mol⁻¹.

As examples of polydimethylsiloxanes of formula (VI), mention may be made of those sold under the names AMS-132, AMS-152, AMS-162, AMS-163, AMS-191 and AMS-1203 by the company Gelest.

According to another variant, formula (VII) is such that Rₐ represents a hydroxyl or (C₁-C₄)alkyl group, such as methyl, ALK¹ represents a (C₅-C₆)alkylene group substituted with an NH group; preferably, ALK¹ represents —(CH₂)₃—N(H)—(CH₂)₂—, and m and n are such that the weight-average molecular mass of the compound of formula (VI) ranges from 5000 g·mol⁻¹ to 500 000 g·mol⁻¹.

As amine polymer, mention may also be made of α,ω-diamino polytetrahydrofurans (or polytetramethylene glycol) and α,ω-diamino polybutadienes.

According to a particular embodiment of the invention, the (poly)amine compounds are chosen from hyperbranched polymers comprising at least one amino group and dendrimers bearing at least one amino group, such as PAMAM polyamidoamine dendrimers with an ethylenediamine core and a terminal amine function.

According to a preferred embodiment, the composition comprises a crosslinking agent R chosen from (poly)amine compounds, in particular chosen from chitosans, aminoalkoxysilanes, polydimethylsiloxanes comprising primary amine groups at the end of the chain or on side chains, amodimethicones, polyglucosamines, spermidine and mixtures thereof.

More preferentially, the composition comprises a crosslinking agent chosen from chitosans, aminoalkoxysilanes and polydialkylsiloxanes comprising primary amine groups at the end of the chain or on side chains such as amodimethicones, and even more preferentially chosen from poly(D-glucosamine), 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, spermidine polydimethylsiloxanes comprising terminal amino groups at the end of the chain, such as bis-cetearyl amodimethicone.

### B) (Poly)Thiol Compounds

According to a preferred embodiment, the crosslinking agent R is chosen from (poly)thiol compounds, also known as “(poly)mercapto” compounds.

The (poly)thiol compound may in particular be organic or inorganic, preferably organic.

In a preferred embodiment, the (poly)thiol compound is silicon-based, i.e. it includes one or more thiol groups and it also includes at least one siloxane chain.

In a particular embodiment, the (poly)thiol compound is inorganic. Mention may be made, for example, of polythiol silicones.

The (poly)thiol compound may in particular be chosen from non-polymeric (poly)thiol compounds.

For the purposes of the present invention, the term “non-polymeric compounds” means compounds which are not directly obtained via a monomer polymerization reaction.

According to one embodiment of the invention, the (poly)thiol compound(s) are organic, non-polymeric and of formula (VIII) below and also the solvates thereof such as hydrates:

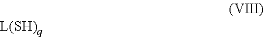

in which formula (VIII):
q denotes an integer greater than or equal to 2; preferably, q is between 2 and 10 inclusive and preferably between 2 and 5;
L denotes a linear or branched, saturated or unsaturated, or (hetero) cyclic, saturated or unsaturated, multivalent (at least divalent) group, in particular comprising between 1 and 500 carbon and/or silicon atoms, more particularly between 2 and 40 carbon and/or silicon atoms, even more particularly between 3 and 30 carbon and/or silicon atoms, preferably between 6 and 20 carbon atoms; L being optionally interrupted and/or terminated with one or more heteroatoms or groups chosen from O, S, N, Si, C(X), and combinations thereof such as —O—, —O—C(X)—, —N(R)—C(X)—, —Si(R_{c})(Rₐ)—O— with R representing a hydrogen atom or a (C₁-C₆)alkyl group such as methyl; and/or L being optionally substituted with one or more groups chosen from: —N(Rₐ) R_{b} and —(X′)ₐ—C(X)—(X″)_{b}—Rₐ; with X, X′ and X″, which may be identical or different, representing an oxygen or sulfur atom, or a group N(R_{b}); a and b being 0 or 1, preferably the sum of a+b being 1; Rₐ and R_{b}, which may be identical or different, represent a hydrogen atom or a (C₁-C₆)alkyl or aryl(C₁-C₄)alkyl group, such as benzyl, preferably Rₐ and R_{b} represent a hydrogen atom; and R_{c} and R_{d}, which may be identical or different, represent a (C₁-C₆)alkyl, aryl(C₁-C₄)alkyl or (C₁-C₆)alkoxy group.

According to a particular embodiment of the invention, the (poly)thiol compound(s) are chosen from polythiol compounds, notably polythiol compounds comprising from 2 to 20 carbon atoms.

According to a preferred embodiment, the (poly)thiol compound(s) are non-polymeric and notably of formula (VIII) defined above, in which q is an integer greater than or equal to 2, preferably q is an integer between 2 and 10 inclusive and preferably between 2 and 5.

The (poly)thiol compounds that are suitable for use in the invention are preferably dithiol compounds.

Preferably, L denotes a C₈-C₁₈ multivalent radical, which is notably linear. Preferentially, the liposoluble polythiol is a notably linear C₈-C₁₈ dithiol. Preferably, the C₈-C₁₈ chain is a hydrocarbon-based chain, i.e. formed from carbon and hydrogen. In particular, the liposoluble polythiol is a linear C₈-C₁₆ and notably C₁₀-C₁₄ dithiol. As (poly)thiol compounds of formula (VII), mention may be made more particularly of 1,8-octanedithiol, 1,10-decanedithiol, 1,12-dodecanedithiol, 1,14-tetradecanedithiol, 1,16-hexadecanedithiol and 1,18-octadecanedithiol. Use is preferably made of 1,10-decanedithiol, 1,12-dodecanedithiol and/or 1,14-tetradecanedithiol, preferentially1,12-dodecanedithiol.

According to another particular embodiment of the invention, the (poly)thiol compound(s) are chosen from thiolated alkoxysiloxanes, such as those of formula (VIII′) below:

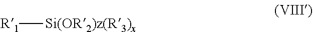

in which formula (VIII′):
R′₁ is a linear or branched, saturated or unsaturated, cyclic or acyclic C₁-C₁₂ hydrocarbon-based chain substituted with one or more groups chosen from thiol groups; and aryl, aryloxy, arylthio, arylamino, the aryl group being substituted with one or more thiol groups, or thiol (C₁-C₆)alkyl, preferably thiol (C₁-C₆)alkyl; and R′₁ is optionally interrupted in its hydrocarbon-based chain with one or more heteroatoms such as O, S, N, a carbonyl group C(O), or a combination thereof such as ester —C(O)—O—, or amide —C(O)—N(H)—, R′₁ being bonded to the silicon atom directly via a carbon atom,
R′₂ and R′₃, which may be identical or different, represent a linear or branched alkyl group comprising from 1 to 6 carbon atoms, preferably from 1 to 4 carbon atoms, such as methyl,
z denotes an integer ranging from 1 to 3, and
x denotes an integer ranging from 0 to 2,
   with z+x=3.

Preferably, R′₂ represents a linear or branched, preferably linear, alkyl group comprising from 1 to 4 carbon atoms, such as ethyl.

Preferably, R′₃ represents a linear or branched, preferably linear, alkyl group comprising from 1 to 4 carbon atoms, such as methyl or ethyl.

Preferably, R′₁ is an acyclic chain, in particular R′₁ is a linear or branched, saturated or unsaturated, preferably saturated, C₁-C₆ hydrocarbon-based chain substituted with one or more thiol groups, preferably substituted with one thiol group.

Preferably, R′₁ is a saturated linear C₁-C₆ hydrocarbon-based chain substituted with a thiol group, and R′₂ represents an alkyl group comprising from 1 to 4 carbon atoms.

Preferably, R′₃ represents an alkyl group comprising from 1 to 4 carbon atoms.

Preferably, z is equal to 3.

According to a more particular embodiment of the invention, the thiolated alkoxysiloxanes are chosen from those of formula (IX) below:

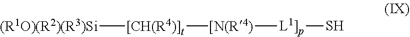

in which formula (IX):
p is 0 or 1;
t is an integer between 1 and 4, preferably 2;
R¹ represents a (C₁-C₆)alkyl radical;
R² and R³, which may be identical or different, preferably identical, are chosen from a (C₁-C₆)alkyl group, in particular a C₁-C₄ alkyl group, such as methyl, and a (C₁-C₆)alkoxy group, in particular a (C₁-C₄)alkoxy group, such as methoxy;
R⁴ and R′⁴, which may be identical or different, represent a hydrogen atom or a (C₁-C₆)alkyl group, such as methyl;
L¹ represents a divalent, saturated, linear or branched C₁-C₂₀ hydrocarbon-based radical.

According to a particular embodiment of the invention, the thiolated alkoxysiloxanes are chosen from those of formula (IX′) below:

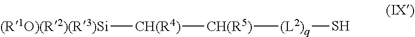

in which formula (IX′):
q is equal to 0 or 1;
X represents an oxygen or sulfur atom, preferably a sulfur atom;
R′¹ denotes a (C₁-C₆)alkyl radical;
R′² and R′³, which may be identical or different, preferably identical, are chosen from a (C₁-C₆)alkoxy group, in particular a C₁-C₄ alkoxy group, and a (C₁-C₆)alkyl radical;
R⁵ represents a hydrogen atom or a C₁-C₄ alkyl group optionally substituted with an amino, thiol or hydroxyl group;
R⁴ represents a hydrogen atom or a C₁-C₄ alkyl group, in particular methyl;
L² represents a linear or branched, saturated C₁-C₂₀ divalent hydrocarbon-based group, optionally interrupted with a heteroatom such as —N(H)—, and/or optionally substituted with one or more hydroxyl, thiol or amino groups.

Preferably, the thiolated alkoxysilane(s) are chosen from 4-(trimethoxysilyl)-1-butanol, 3-(trimethoxysilyl)-1-propanol, 3-(triethoxysilyl)-1-propanol, 11-(trimethoxysilyl)-1-undecanethiol, 4-(trimethoxysilyl)-2-butanethiol, 2-(triethoxysilyl) ethanethiol, 3-(triethoxysilyl)-1-propanethiol, 2-(trimethoxysilyl) ethanethiol, 3-(trimethoxysilyl)-1-propanethiol and 3-(dimethoxymethylsilyl)-1-propanethiol.

More preferentially, the thiolated alkoxysilane(s) e chosen from 2-(triethoxysilyl) ethanethiol (18236-15-2) and 3-(triethoxysilyl)-1-propanethiol (14814-09-6).

According to a preferred embodiment of the invention, the (poly)thiol compound(s) are chosen from polymeric (poly)thiol compounds.

The polymeric (poly)thiol compounds may be star, comb, brush and dendritic homopolymers or copolymers bearing thiol units. The polymers may be of natural origin such as polysaccharides or polypeptides, or of synthetic origin such as acrylic polymers, polyesters or polyglycols. The thiol units may be present as terminal and/or side groups.

Examples that may be mentioned include the polymers described in the following articles: Polymers containing groups of biological activity, C. G. Overberger et al., Polytechnic Institute of Brooklyn, http://pac.iupac.org/publications/pac/pdf/1962/pdf/0402x0521.pdf; EP 1 247 515 A2; U.S. Pat. No. 3,676,440; and EP 1 572 778.

The polymeric (poly)thiol compounds of the invention are preferably organic and/or silicone compounds, more preferentially of formula (X):

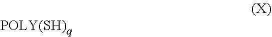

in which formula (X):
q is greater than or equal to 2, preferably greater than or equal to 3;
POLY denotes a polymer-based radical, preferably carbon-based or silicone-based; POLY being optionally interrupted with one or more heteroatoms or groups chosen from O, S, N, Si, C(X), and combinations thereof such as —O—, —O—C(X)—, —N(R)—C(X)—, —Si(R_{c}) (Rₐ)—O— with R representing a hydrogen atom or a (C₁-C₆)alkyl group such as methyl; and/or POLY being optionally substituted with one or more halogen atoms, or a group chosen from Rₐ (R_{b})N— and —(X′)ₐ—C(X)—(X″)_{b}—Rₐ; X, X′ and X″, which may be identical or different, represent an oxygen or sulfur atom or a group N(R_{b}); a and b being 0 or 1, preferably the sum of a+b being 1; Rₐ and R_{b}, which may be identical or different, represent a hydrogen atom or a (C₁-C₁₀)alkyl or aryl(C₁-C₄)alkyl group, such as benzyl, preferably Rₐ and R_{b} represent a hydrogen atom; and R_{c} and R_{d}, which may be identical or different, represent a (C₁-C₁₀)alkyl, aryl(C₁-C₄)alkyl or (C₁-C₁₀)alkoxy group.

The methods for preparing the polymeric (poly)thiol compounds used according to the invention are known to those skilled in the art; several methods are reported hereinbelow in a non-limiting manner. The polymeric (poly)thiol compounds used according to the invention may be obtained by polymerization or polycondensation of monomer units bearing thiol or protected thiol functions, optionally as a copolymerization or co-polycondensation of monomer units free of thiol or protected thiol functions.

According to one embodiment of the invention, the polymeric (poly)thiol compounds used according to the invention are polymers which are soluble in cosmetic media, particularly in aqueous or aqueous-alcoholic media. They are more preferentially obtained from amino polymers and the ammonium salts thereof or from polyhydroxylated polymers.

According to another embodiment of the invention, the thiolated polymers used according to the invention are polymers that are soluble in lipophilic media.

According to one embodiment of the invention, the polythiol compound is a polymeric compound of formula (X) in which q denotes an integer greater than or equal to 2, and POLY denotes a carbon-based and/or silicon-based, preferably silicon-based, polymeric radical,

POLY also possibly containing one or more heteroatoms chosen from O, N or S, and/or one or more functions chosen from (thio)ester, (thio)ketone, (thio)amide, (thio)urea and (thio)carbamate functions, and/or possibly being substituted with one or more linear or branched (C₁-C₁₀)alkyl or linear or branched (C₁-C₁₀)alkoxy groups, it being understood that when POLY is substituted, the thiol functions may be borne by the substituent(s).

The weight-average molecular weight of the polythiol polymer compounds, such as those of formula (X), is generally between 500 and 400 000 g·mol⁻¹, preferably between 500 and 150 000 g·mol⁻¹.

According to a particular embodiment of the invention, the polythiol compounds are chosen from polyorganosiloxanes including thiol groups on end chains, such as those of formula (XI) below:

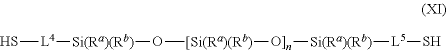

in which formula (XI):
R^{a} and R^{b}, which may be identical or different, preferably identical, represent a group from among: (C₁-C₄)alkyl such as methyl, (C₁-C₄)alkoxy such as methoxy, aryl such as phenyl, aryloxy such as phenoxy, aryl(C₁-C₄)alkyl such as benzyl, or aryl(C₁-C₄)alkoxy such as benzoxy, preferably (C₁-C₄)alkyl such as methyl;
n represents an integer greater than or equal to 1 and more particularly the value of n is such that the weight-average molecular weight of the silicone ranges from 500 to 55 000 g·mol⁻¹; in particular, n is an integer ranging from 1 to 100, preferably ranging from 5 to 50 and preferentially ranging from 10 to 30, and
L⁴ and L⁵, which may be identical or different, preferably identical, represent a linear or branched, saturated or unsaturated, optionally cyclic hydrocarbon-based chain comprising from 1 to 100 carbon atoms, optionally interrupted with one or more heteroatoms such as oxygen, sulfur or nitrogen, in particular oxygen, and in particular represent a covalent bond or a (C₁-C₆)alkylene, (C₁-C₆)alkylenoxy, oxy(C₁-C₆)alkylene, (C₁-C₆)alkylenoxy(C₁-C₆)alkylene, (C₁-C₆)alkylenoxy(C₁-C₆)alkylenoxy or oxy(C₁-C₆)alkylenoxy(C₁-C₆)alkylene group, preferably a (C₁-C₆)alkylene, (C₁-C₆)alkylenoxy, oxy(C₁-C₆)alkylene or (C₁-C₆)alkylenoxy(C₁-C₆)alkylene group.

Preferentially, the (poly)thiol compounds are polythiol polyorganosiloxanes, more preferentially polythiol polydimethylsiloxanes, notably chosen from those of formula (XII):

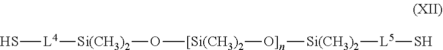

in which formula (XII):
L⁴ and L⁵ are as defined previously in formula (IX), in particular L⁴ and L⁵ represent a (C₁-C₆)alkylene, (C₁-C₆)alkylenoxy, oxy(C₁-C₆)alkylene or (C₁-C₆)alkylenoxy(C₁-C₆)alkylene group, more preferentially a divalent group chosen from —R₂—, —O—R₂—, —R₂—O— and —R₂—O—R₂—, preferably —R₂—O—R₂—, with R₂ representing a linear or branched, preferably linear, (C₂-C₆)alkylene group, such as ethylene or n-propylene, preferably n-propylene; and
n is as defined in formula (XI).

As polythiol compounds of formula (XII), mention may be made of mercaptosiloxanes or thiolated siloxanes in which the thiol functions are at the chain ends, sold by the company Shin-Etsu under the reference X-22-167B, and mercaptosiloxane in which the mercapto functions are pendent, sold by the company Shin-Etsu under the reference KF-2001, or polydimethylsiloxanes in which the thiol functions are at the chain ends, via thio-n-propyl, 80-120 groups, sold by the company Gelest under the name DMS-SM 21.

Preferentially, the polythiol compounds are chosen polyorganosiloxanes including thiol groups on side chains, such as those of formula (XIII):

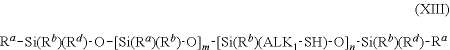

in which formula (XIII):
R^{a} and R^{b} are as defined in formula (XI) and R^{d} is as defined for R^{a} and R^{b}, preferably R^{a}, R^{b} and R^{d}, which are identical, represent a (C₁-C₆)alkyl group, such as methyl;
R^{d} may also represent a (C₁-C₆)alkyl group substituted with a (C₁-C₄)alkylamino or amino or thiol group, preferably (C₁-C₄)alkyl such as methyl;
ALK₁ represents a linear or branched, optionally cyclic, saturated or unsaturated divalent hydrocarbon-based chain comprising from 1 to 100 carbon atoms, optionally interrupted with one or more heteroatoms such as oxygen, sulfur or nitrogen, in particular oxygen, a (thio)carbonyl group C(X) with X representing O or S, or combinations thereof such as —O—, —O—C(O)— or —C(O)—O—; preferably, ALK₁ represents a (C₁-C₆)alkylene and more preferentially (C₁-C₄)alkylene group such as propylene;
n and m, which may be identical or different, represent an integer greater than 2 and more particularly the values of m and n are such that the weight-average molecular weight of said polyorganosiloxane is between 1000 and 55 000 g·mol⁻¹.

As examples of polythiol compounds of formula (XIII), mention may be made of those sold by the company Genesee Polymers under the names GP-367, GP-71-SS, GP-800 and GP-710s, preferably GP-367, sold by the company Genesee Polymers.

The polythiol compounds are notably polydimethylsiloxanes including at least two thiol groups, for instance the products SMS-022, SMS-042 and SMS-992 sold by the company Gelest in https://www.gpcsilicones.com/products/silicone-fluids/mercapto-functional, https://www.shinetsusilicone-global.com/products/type/oil/detail/search/deg07.shtml, and 1053_Reactive Silicones_Silanes/Silicones-Gelest.

According to a particular embodiment of the invention, the (poly)thiol compounds are chosen from hyperbranched polymers comprising at least one thiol group and dendrimers bearing at least one thiol group, such as thiolated PAMAM dendrimers.

Preferably, the (poly)thiol compounds used according to the invention are chosen from polydiallylsiloxanes, notably polydimethylsiloxanes, including at least two thiol groups such as those of formula (XIII).

### C) (Poly)Acrylate Compounds

According to a particular embodiment, the crosslinking agent is a (poly)acrylate compound.

The term “(poly)acrylate” means a compound which comprises at least one acrylate ester group H₂C═C(R^{e})—C(O)—Y— with R^{e} representing a hydrogen atom or a (C₁-C₄)alkyl group, such as methyl, preferably R^{e} representing a hydrogen atom, and Y representing an oxygen atom or an amino group —N(H)—, preferably an oxygen atom.

More particularly, the (poly)acrylate(s) of the invention are of formula (XIV):

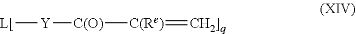

in which formula (XIV) q and L are as defined in formula (VIII), Y and R^{e} being as defined previously, preferably Y=O and R^{e}=H.

According to a preferred embodiment, the compounds of formula (XIV) are such that L represents a di- or trivalent, preferably trivalent, hydrocarbon-based chain comprising from 1 to 8 carbon atoms, q is 2 or 3, preferably 3, Y represents O, and R^{e} represents a hydrogen atom.

According to a particular embodiment, the (poly)acrylate compounds are chosen from polyorganosiloxanes including at least one acrylate group on the side chain, such as those of formula (XV):

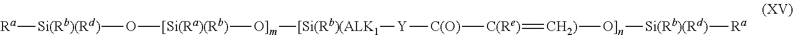

in which formula (XV):
R^{a}, R^{b} and R^{d} are as defined for formula (XIII), preferably R^{a}, R^{b} and R^{d} represent a (C₁-C₆)alkyl group, such as methyl,
ALK₁ is as defined for formula (XIII), preferably ALK₁ represents a (C₁-C₆)alkylene group, more preferentially a (C₁-C₄)alkylene group, such as propylene,
n and m, which may be identical or different, represent an integer greater than 2 and more particularly the values of m and n are such that the weight-average molecular weight of said polyorganosiloxane is between 1000 and 55 000 g·mol⁻¹.
Y is as defined previously, and is preferably an oxygen atom.

More particularly, the (poly)acrylate compound may be chosen from 1,3-butanediol diacrylate, 1,4-butanediol diacrylate, bis(trimethylolpropane) tetraacrylate, glyceryl 1,3-diglycerolate diacrylate, glyceryl propoxylate (1PO/OH) triacrylate, 1,6-hexanediol diacrylate, 1,6-hexanediol ethoxylate diacrylate, hydroxypivalyl hydroxypivalate, neopentyl glycol diacrylate, neopentyl glycol propoxylate (1PO/OH) diacrylate, pentaerythrityl tetraacrylate, pentaerythrityl triacrylate, poly(propylene glycol) diacrylate, tricyclo[5.2.1.02,6]decanedimethanol diacrylate, trimethylolpropane ethoxylate (1EO/OH) methyl ether diacrylate, trimethylolpropane propoxylate triacrylate, trimethylolpropane triacrylate, trimethylolpropane trimethacrylate, tri(propylene glycol) diacrylate, and tris[2-(acryloyloxy)ethyl] isocyanurate.

The (poly)acrylate compound may also be chosen from N,N′-methylenebisacrylamide. According to this embodiment, the (poly)acrylate compound is combined in its implementation with an amine catalyst as described, for example, in Progress in coating 129, 21-25 (2019) and Progress in coating 135, 510-516 (2019). Preferably, the amine catalyst(s) are chosen from piperidine, DMAP (dimethylaminopyridine), DBU (1,8-diazabicyclo[5.4.0]undec-7-ene), DABCO (1,4-diazabicyclo[2.2.2]octane) and DBN (1,5-diazabicyclo[4.3.0]non-5-ene), more preferentially chosen from DBU (1,8-diazabicyclo[5.4.0]undec-7-ene), DABCO (1,4-diazabicyclo[2.2.2]octane) and DBN (1,5-diazabicyclo[4.3.0]non-5-ene), and in particular the catalyst is DBU (1,8-diazabicyclo[5.4.0]undec-7-ene).

More particularly, the (poly)acrylate compounds are chosen from those of formula (XIV), notably trimethylolpropane triacrylate, and those of formula (XV), notably the copolymers of dimethylsiloxane and of acryloxypropylmethylsiloxane.

### D) Metal Alkoxides

According to a particular embodiment, the crosslinking agent R is a compound chosen from the metal alkoxides of formulae (XIVₐ), (XIV_{b}), (XIV_{c}) and (XIVₐ) below and mixtures thereof:

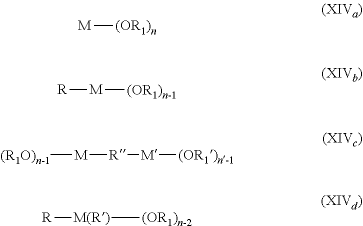

in which formulae (XIVₐ), (XIV_{b}), (XIV_{c}) and (XIVₐ):
M and M′, which may be identical or different, represent an atom chosen from alkaline-earth metals, transition metals, metals of the lanthanide family, post-transition metals such as aluminium or tin and metalloids such as boron; preferably transition metals such as Ti and post-transition metals such as aluminium;
n and n′ respectively represent the valencies of the atoms represented by M and M′;
R₁ and R₁′, which may be identical or different, represent a linear or branched, saturated or unsaturated hydrocarbon-based group containing from 1 to 30 carbon atoms, preferably from 1 to 6 carbon atoms, said hydrocarbon-based group being optionally interrupted with 1 to 20 heteroatoms chosen from O, N, S and P, notably O or N; and/or said hydrocarbon-based group being optionally substituted with one or more hydroxyl or carbonyl groups;
R and R′, which may be identical or different, represent a hydrogen atom or a linear, branched, acyclic or cyclic, saturated or unsaturated hydrocarbon-based group containing from 1 to 30 carbon atoms, preferably from 2 to 20 carbon atoms, optionally interrupted with 1 to 20 heteroatoms chosen from O, N, S and/or P, notably O or N, and/or said hydrocarbon-based group being optionally substituted with one or more hydroxyl or carbonyl groups;
R″ represents —O—, —N(R2)-, —S— or a linear, cyclic or branched, saturated or unsaturated divalent hydrocarbon-based group containing from 1 to 30 carbon atoms, preferably from 2 to 20 carbon atoms, optionally interrupted with 1 to 20 heteroatoms chosen from O, N, S and P, notably O or N, with R2 representing a linear, cyclic or branched, saturated or unsaturated hydrocarbon-based group containing from 1 to 30 carbon atoms, preferably from 2 to 20 carbon atoms.

Preferably, M and M′, which may be identical or different, represent an atom chosen from transition metals such as titanium or zirconium or alkaline-earth metals such as magnesium, more preferentially chosen from transition metals such as titanium or zirconium, even more preferentially titanium.

Preferably, the organometallic compound(s) are chosen from the alkoxides of formula (XIVₐ) as defined previously. According to this preferred embodiment, the organometallic compound(s) are more particularly chosen from the alkoxides of formula (XIVₐ), in which M represents an atom chosen from transition metals, metals of the lanthanide family, post-transition metals, such as aluminium, tin, metalloids, such as boron, or alkaline-earth metals, such as magnesium or calcium; n represents the valency of the atom represented by M; R₁ represents a saturated, linear or branched, hydrocarbon-based group containing from 1 to 30 carbon atoms, preferably from 1 to 6 carbon atoms.

According to another more preferred embodiment, the organometallic compound(s) are chosen from the alkoxides of formula (XIVₐ), in which M represents an atom chosen from transition metals, such as zirconium or titanium, metals of the lanthanide family, post-transition metals, such as aluminium or tin, metalloids, such as boron, and alkaline-earth metals, such as magnesium, preferably M represents a titanium atom; n represents the valency of the atom represented by M, notably 1, 2, 3 or 4, in particular 4; R₁ represents a methyl, ethyl, 2-ethylhexyl, propyl, isopropyl, n-butyl, isobutyl or t-butyl group.

According to an even more preferred embodiment, the organometallic compound(s) are chosen from zirconium ethoxide (Zr(OC₂H₅)₄), zirconium propoxide (Zr(OCH₂CH₂CH₃)₄), zirconium isopropoxide (Zr(OCH(CH₃)₂)₄), zirconium butoxide Zr(OCH₂CH₂CH₂CH₃)₄, zirconium tert-butoxide (Zr(OC(CH₃)₃)₄), titanium ethoxide (Ti(OC₂H₅)₄), titanium propoxide (Ti(OCH₂CH₂CH₃)₄), titanium isopropoxide (Ti(OCH(CH₃)₂)₄), titanium butoxide (Ti(OCH₂CH₂CH₂CH₃)₄), titanium tert-butoxide (Ti(OC(CH₃)₃)₄), titanium 2-ethylhexyloxide (Ti(OCH₂CH(C₂H₅)(CH₂)₃CH₃)₄), and mixtures thereof, more preferentially chosen from zirconium propoxide, titanium propoxide, titanium butoxide and mixtures thereof.

More preferentially, the crosslinking agent R is a compound of formula (XIVₐ) preferably in which M represents an atom chosen from transition metals, notably titanium such as titanium butoxide.

### E) (Poly)Carbonyl Compounds

According to a particular embodiment, the crosslinking agent R is a (poly)carbonyl compound.

In particular, the (poly)carbonyl compound is chosen from terephthalaldehyde, 5,5-dimethyl-1,3-cyclohexanedione, phenylglyoxal, isophthalaldehyde, 4-acetylbenzaldehyde, 4,4-diformyltriphenylamine, 2-acetylbenzaldehyde, 3-(2-furoyl)quinoline-2-carboxaldehyde, 3-(2-furoyl)quinoline-2-carboxaldehyde, 3-acetylbenzaldehyde, 9-(2-ethylhexyl) carbazole-3,6-dicarboxaldehyde, phthaldialdehyde, 1,3-cyclohexanedione, 4,4′-biphenyldicarboxaldehyde, benzene-1,3,5-tricarboxaldehyde, and nonionic or anionic oxidized polysaccharides such as oxidized inulins, notably those of formula (II) as defined hereinbelow. In particular, the (poly)carbonyl compounds include a saturated or unsaturated, aromatic C₅-C₇ carbocycle, preferably aromatic, such as phenyl, or non-aromatic and saturated such as cyclohexyl, more preferentially unsaturated and aromatic, such as terephthaldehyde.

According to a particular embodiment, the (poly)carbonyl compound(s) are chosen from nonionic or anionic oxidized polysaccharides comprising one or more aldehyde groups, and optionally one or more anionic groups.

These anionic groups are preferably carboxyl or carboxylate groups.

The nonionic or anionic oxidized polysaccharides according to the invention may be represented by formula (II) below:

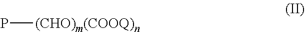

in which formula (II):
P represents a polysaccharide chain preferably consisting of monosaccharides comprising 5 carbon atoms or more than 5 carbon atoms, preferably 6 or more than 6 carbon atoms and more particularly 6 carbon atoms;
Q is chosen from a hydrogen atom, the ions derived from an alkali metal or an alkaline-earth metal such as sodium or potassium, ammonia, organic amines such as monoethanolamine, diethanolamine, triethanolamine and 3-amino-1,2-propanediol and basic amino acids such as lysine, arginine, sarcosine, ornithine and citrulline;
m+n is greater than or equal to 1;
m is such that the degree of substitution of the polysaccharide with one or more aldehyde groups (DS(CHO)) is within the range from 0.001 to 2 and preferably from 0.005 to 1.5;
n is such that the degree of substitution of the polysaccharide with one or more carboxylic groups (DS(COOX)) is within the range from 0 to 2 and preferably from 0.001 to 1.5.

The expression “degree of substitution DS(CHO) or DS(COOX) of the polysaccharides according to the invention” means the ratio between the number of carbons oxidized to give an aldehyde or carboxylic group for all the repeating units and the number of elementary monosaccharides (even opened by preoxidation) constituting the polysaccharide. The groups CHO and COOX may be obtained during the oxidation of certain carbon atoms, for example on the carbon atoms 2, 3 or 6, of a saccharide unit containing 6 carbon atoms.

Preferably, the oxidation may take place on carbons 2 and 3, more particularly of 0.01% to 75% by number, and preferably of 0.1% to 50% by number of the rings that may have been opened.

The polysaccharide chain, represented by P, is preferably chosen from celluloses, starches, maltodextrins, guar gums, xanthan gums, pullulan gums, agar-agar gums, carrageenan gums, gellan gums, acacia gums, polyxylans and tragacanth gums, and derivatives thereof.

The term “derivative” means the compounds obtained by chemical modification of the mentioned compounds. They may be esters, amides or ethers of said compounds.

The oxidation may take place according to a process known in the art, for example according to the process described in FR 2 842 200, in document FR 2 854 161 or in the article “Hydrophobic films from maize bran hemicelluloses” by E. Fredon et al., Carbohydrate Polymers 49, pages 1 to 12 (2002).

Another oxidation process is described in the article “Water soluble oxidized starches by peroxide reaction extrusion” Industrial Crops and Products 7, R. E. Wing, J. L. Willet, 45-52 (1997).

According to this embodiment, the (poly)carbonyl compound is combined in its implementation with an amine catalyst as described in the articles Progress in coating 129, 21-25 (2019) and Progress in coating 135, 510-516 (2019). Preferably, the amine catalyst(s) are chosen from piperidine, DMAP (dimethylaminopyridine), DBU (1,8-diazabicyclo[5.4.0]undec-7-ene), DABCO (1,4-diazabicyclo[2.2.2]octane) and DBN (1,5-diazabicyclo[4.3.0]non-5-ene), more preferentially chosen from DBU (1,8-diazabicyclo[5.4.0]undec-7-ene), DABCO (1,4-diazabicyclo[2.2.2]octane) and DBN (1,5-diazabicyclo[4.3.0]non-5-ene), and in particular the catalyst is DBU (1,8-diazabicyclo[5.4.0]undec-7-ene).

According to a preferred embodiment, the crosslinking agent is chosen from (poly)amine compounds A), (poly)thiol compounds B), (poly)acrylate compounds C), metal alkoxides D) and (poly)carbonyl compounds E), and preferably from (poly)amine compounds A).

In particular, said (poly)amine compounds A) are chosen from a) chitosans, such as poly(D-glucosamine), b) polyether diamines, particularly polyethylene glycol α,ω-diamine (bearing an amine function at the end of the chain), c) polyether triamines, such as polyetheramine (or Jeffamine), d) aminoalkoxysilanes, such as APTES, and e) polydialkylsiloxanes comprising primary amine groups at the end of the chain or on side chains, in particular polydimethylsiloxanes comprising primary amine groups, such as bis(3-aminopropyl)-terminated poly(dimethoxysiloxane) (PDMS-diNH₂) and amodimethicones comprising amine groups on side chains, such as bis-cetearyl amodimethicone, notably the product sold by Momentive Performance Materials.

According to a preferred embodiment, said (poly)amine compounds A) are chosen from a) chitosans, such as poly(D-glucosamine), c) polyether triamines, such as polyetheramine (or Jeffamine), and e) polydialkylsiloxanes comprising primary amine groups at the end of the chain or on side chains, in particular polydimethylsiloxanes comprising primary amine groups, such as bis(3-aminopropyl)-terminated poly(dimethoxysiloxane) (PDMS-diNH₂) and amodimethicones comprising amine groups on side chains, such as bis-cetearyl amodimethicone, notably the product sold by Momentive Performance Materials.

In particular, said (poly)thiol compounds B) are chosen from a) polydialkylsiloxanes bearing thiol functions, and b)alkoxysilanes bearing thiol functions, and in particular are chosen from a) polydialkylsiloxanes bearing thiol functions, preferentially polydimethylsiloxanes comprising thiol groups on the side chain (such as mercaptopropyl), notably those of formula (XIII).

In particular, said (poly)acrylate C) compounds are chosen from those of formula (XIV), notably trimethylolpropane triacrylate, and those of formula (XV), notably copolymers of dimethylsiloxane and acryloxypropylmethylsiloxane, preferably trimethylolpropane triacrylate.

According to a preferred embodiment, the crosslinking agent(s) is (are) chosen from:

A) (poly)amine compounds chosen from:
ia) chitosans such as poly(D-glucosamine),
ib) polyether diamines, in particular polyethylene glycol α,ω-diamines, bearing a chain-end amine function,
ic) polyether triamines, in particular polyetheramines (or Jeffamine),
id) aminoalkoxysilanes, in particular APTES,
ie) 4,7,10-trioxa-1,13-tridecanediamine, and
if) polydialkylsiloxanes comprising primary amine groups at the end of the chain or on side chains, in particular polydimethylsiloxanes comprising primary amine groups, more particularly bis(3-aminopropyl)-terminated poly(dimethoxysiloxane) (PDMS-diNH₂) and amodimethicones comprising amine groups on side chains, more particularly bis-cetearyl amodimethicone;
   B) (poly)thiol compounds chosen from:
iia) polydialkylsiloxanes bearing thiol functions, and
iib)alkoxysilanes bearing thiol functions,
and in particular chosen from iia) polydialkylsiloxanes bearing thiol functions, preferably from polydimethylsiloxanes comprising thiol groups on the side chain, in particular mercaptopropyl, and more particularly chosen from the compounds of formula (XIII):

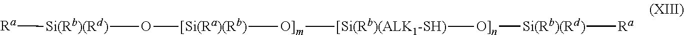

in which formula (XIII):
R^{a} and R^{b}, which may be identical or different, preferably identical, represent a (C₁-C₄)alkyl group, in particular methyl, a (C₁-C₄)alkoxy group, in particular methoxy, an aryl group, in particular phenyl, an aryloxy group, in particular phenoxy, an aryl(C₁-C₄)alkyl group, in particular benzyl, or an aryl(C₁-C₄)alkoxy group, in particular benzoxy, preferably a (C₁-C₄)alkyl group, more preferentially methyl,
R^{d} represents a (C₁-C₄)alkyl group, in particular methyl, a (C₁-C₄)alkoxy group, in particular methoxy, an aryl group, in particular phenyl, an aryloxy group, in particular phenoxy, an aryl(C₁-C₄)alkyl group, in particular benzyl, an aryl(C₁-C₄)alkoxy group, in particular benzoxy, or a (C₁-C₆)alkyl group substituted with a (C₁-C₄)alkylamino, amino or thiol group, and preferably a (C₁-C₄)alkyl group, more preferentially methyl, and preferably R^{a}, R^{b} and R^{d} are identical and represent a (C₁-C₆)alkyl group, more preferentially methyl,
ALK₁ represents a linear or branched, optionally cyclic, saturated or unsaturated divalent hydrocarbon-based chain comprising from 1 to 100 carbon atoms, optionally interrupted with one or more heteroatoms such as oxygen, sulfur or nitrogen, in particular oxygen, a (thio) carbonyl group C(X) with X representing O or S, or combinations thereof, in particular —O—, —O—C(O)— or —C(O)—O—; preferably, ALK₁ represents a (C₁-C₆)alkylene and more preferentially (C₁-C₄)alkylene group, even more preferentially propylene,
n and m, which may be identical or different, represent an integer greater than 2, and in particular the values of m and n are such that the weight-average molecular weight of said polyorganosiloxane is between 1000 and 55 000 g·mol⁻¹; and
   C) the (poly)acrylate compounds of formula (XIV):

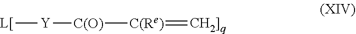

in which formula (XIV):
q represents an integer greater than or equal to 2, in particular n is between 2 and 10 inclusive and preferably between 2 and 5,
L denotes a linear or branched, saturated or unsaturated, or (hetero) cyclic, saturated or unsaturated, multivalent (at least divalent) group, in particular comprising between 1 and 500 carbon and/or silicon atoms, more particularly between 2 and 40 carbon and/or silicon atoms, even more particularly between 3 and 30 carbon and/or silicon atoms, preferably between 6 and 20 carbon atoms; L being optionally interrupted and/or terminated with one or more heteroatoms or groups chosen from O, S, N, Si, C(X), and combinations thereof, in particular —O—, —O—C(X)—, —N(R)—C(X)—, —Si(R_{c})(Rₐ)—O— with R representing a hydrogen atom or a (C₁-C₆)alkyl group, in particular methyl; and/or L being optionally substituted with one or more groups chosen from: —N(Rₐ)R_{b} and —(X′)ₐ—C(X)—(X″)_{b}—Rₐ; with X, X′ and X″, which may be identical or different, representing an oxygen or sulfur atom, or a group N(R_{b}); a and b being 0 or 1, preferably the sum of a+b being 1; Rₐ and R_{b}, which may be identical or different, represent a hydrogen atom, a (C₁-C₆)alkyl group or an aryl(C₁-C₄)alkyl group, in particular benzyl, preferably Rₐ and R_{b} represent a hydrogen atom, and R_{c} and Rₐ, which may be identical or different, represent a (C₁-C₆)alkyl, aryl(C₁-C₄)alkyl or (C₁-C₆)alkoxy group,
Rₑ represents a hydrogen atom or a (C₁-C₄)alkyl group, in particular methyl; preferably, Rₑ represents a hydrogen atom, and
Y represents an oxygen atom or an amino group —N(H)—, preferably an oxygen atom, preferably Y is an oxygen atom and Rₑ is a hydrogen atom, preferably L represents a di- or trivalent, preferably trivalent, hydrocarbon-based chain comprising from 1 to 8 carbon atoms, q is 2 or 3, preferably 3, and
   more preferentially, the compounds of formula (XIV) are trimethylolpropane triacrylate;
   D) metal alkoxides chosen from: zirconium ethoxide (Zr(OC₂H₅)₄), zirconium propoxide (Zr(OCH₂CH₂CH₃)₄), zirconium isopropoxide (Zr(OCH(CH₃)₂)₄), zirconium butoxide Zr(OCH₂CH₂CH₂CH₃)₄, zirconium tert-butoxide (Zr(OC(CH₃)₃)₄), titanium ethoxide (Ti(OC₂H₅)₄), titanium propoxide (Ti(OCH₂CH₂CH₃)₄), titanium isopropoxide (Ti(OCH(CH₃)₂)₄), titanium butoxide (Ti(OCH₂CH₂CH₂CH₃)₄), titanium tert-butoxide (Ti(OC(CH₃)₃)₄), titanium 2-ethylhexyloxide (Ti(OCH₂CH(C₂H₅)(CH₂)₃CH₃)₄), and mixtures thereof, more preferentially chosen from zirconium propoxide, titanium propoxide, titanium butoxide and mixtures thereof,
   E) (poly)carbonyl compounds chosen from (poly)carbonyl compounds including a saturated or unsaturated, aromatic C₅-C₇ carbocycle, preferably aromatic, such as phenyl, or non-aromatic and saturated such as cyclohexyl, more preferentially unsaturated and aromatic, such as terephthaldehyde.
   Cosmetic Active Agents iii)

According to a particular embodiment, the process of the invention also comprises the application of iii) at least one cosmetic active agent to keratin materials, notably the skin and/or the lips and/or the eyelashes and/or the eyebrows.

More particularly, in the treatment process according to the invention, at least one of the compositions “C1”, “C2”, “C3”, “C4” or “C5” used comprises one or more cosmetic active agents.

In particular, the cosmetic active agent(s) iii) is (are) chosen from:
a) dyestuffs (or colouring agents), in particular chosen from pigments, direct dyes and mixtures thereof,
b) active agents for caring for keratin materials, preferably the skin,
c) UV-screening agents, and
d) mixtures thereof.

According to a particular embodiment, the at least one cosmetic active agent iii) is chosen from dyestuffs, preferably chosen from pigments, direct dyes and mixtures thereof, more preferentially pigments.

According to another embodiment, at least one of the compositions “C1”, “C2”, “C3”, “C4” or “C5” used comprises one or more cosmetic active agent(s) chosen from a) dyestuffs (or colouring agents), in particular chosen from pigments, direct dyes and mixtures thereof, preferably chosen from pigments.

According to a particular embodiment, composition “C1” contains at least one cosmetic active agent iii) as defined previously and hereinbelow, preferably chosen from a) dyestuffs, and more preferentially chosen from pigments.

According to a particular embodiment, composition “C2” contains at least one cosmetic active agent iii) as defined previously and hereinbelow, preferably chosen from a) dyestuffs, and more preferentially chosen from pigments.

According to a particular embodiment, composition “C3” contains at least one cosmetic active agent iii) as defined previously and hereinbelow, preferably chosen from a) dyestuffs, and more preferentially chosen from pigments.

Needless to say, a person skilled in the art will take care to select this or these optional cosmetic active agent(s), and/or the amount thereof, such that the advantageous properties of the corresponding composition according to the invention are not, or are not substantially, adversely affected by the envisaged addition.

### Dyestuffs

According to a particular embodiment, the process of the invention uses one or more dyestuffs.

More particularly, in the process of the invention, at least one of the compositions “C1”, “C2”, “C3”, “C4” or “C5” used comprises at least one particulate or non-particulate, water-soluble or water-insoluble dyestuff, preferably in a proportion of at least 0.01% by weight relative to the total weight of the composition considered.

For obvious reasons, this amount is liable to vary significantly with regard to the intensity of the desired colour effect and of the colour intensity afforded by the dyestuffs under consideration, and its adjustment clearly falls within the competence of a person skilled in the art.

Preferably, at least one of the compositions “C1”, “C2”, “C3”, “C4” or “C5” comprises at least one dyestuff chosen from pigments, direct dyes and mixtures thereof, more preferentially pigments.

More preferentially, the pigment(s) of the invention are chosen from carbon black, iron oxides, in particular yellow, red and black iron oxides, and micas coated with iron oxide, triarylmethane pigments, in particular blue and violet triarylmethane pigments, in particular Blue 1 Lake, azo pigments, in particular red azo pigments, more particularly D&C Red 7, an alkali metal salt of lithol red, in particular the calcium salt of lithol red B, and even more preferentially chosen from red iron oxides, yellow iron oxides and azo pigments, in particular red azo pigments, more particularly D&C Red 7.

### Pigments

For the purposes of the invention, the term “pigment” means any compound that is capable of imparting colour to keratin materials. These compounds have a solubility in water at 25° C. and at atmospheric pressure (760 mmHg) of less than 0.05% by weight, and preferably less than 0.01% by weight.

As pigments that are suitable for use in the invention, mention may notably be made of the organic and/or mineral pigments known in the art, notably those described in Kirk-Othmer's Encyclopedia of Chemical Technology and in Ullmann's Encyclopedia of Industrial Chemistry.

These pigments may be synthetic or natural.

These pigments may be in pigment powder or paste form.

They may be coated or uncoated.

These pigments may be chosen, for example, from mineral pigments, organic pigments, lakes, pigments with special effects such as nacres or glitter flakes, and mixtures thereof.

A pigment that is suitable for use in the invention may be chosen from mineral pigments.

The term “mineral pigment” means any pigment that satisfies the definition in Ullmann's encyclopedia in the chapter on inorganic pigments. Among the mineral pigments that are useful in the present invention, mention may be made of manganese violet, ultramarine blue, chromium hydrate, ferric blue and titanium, zirconium or cerium oxides or dioxides, and also of zinc, iron or chromium oxides.

It may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30. They may also be pigments having a structure that may be, for example, of silica microsphere type containing iron oxide. An example of a pigment having this structure is the product sold by the company Miyoshi under the reference PC Ball PC-LL-100 P, this pigment consisting of silica microspheres containing yellow iron oxide.

Advantageously, the pigments may be iron oxides and/or titanium dioxides.

A pigment that is suitable for use in the invention may be chosen from organic pigments.

The term “organic pigment” means any pigment that satisfies the definition in Ullmann's encyclopaedia in the chapter on organic pigments. Among the organic pigments that are useful in the present invention, mention may be made of nitroso, nitro, azo, xanthene, pyrene, quinoline, anthraquinone, triphenylmethane, fluorane, phthalocyanine, metal-complex, isoindolinone, isoindoline, quinacridone, perinone, perylene, diketopyrrolopyrrole, indigo, thioindigo, dioxazine, triphenylmethane and quinophthalone compounds. In particular, the white or coloured organic pigments may be chosen from carmine, carbon black, aniline black, azo yellow, quinacridone, phthalocyanine blue, the blue pigments codified in the Color Index under the references CI 42090, 69800, 69825, 74100, 74160, the yellow pigments codified in the Color Index under the references CI 11680, 11710, 19140, 20040, 21100, 21108, 47000, 47005, the green pigments codified in the Color Index under the references CI 61565, 61570, 74260, the orange pigments codified in the Color Index under the references CI 11725, 45370, 71105, the red pigments codified in the Color Index under the references CI 12085, 12120, 12370, 12420, 12490, 14700, 15525, 15580, 15620, 15630, 15800, 15850, 15865, 15880, 26100, 45380, 45410, 58000, 73360, 73915, 75470, the pigments obtained by oxidative polymerization of indole or phenol derivatives as described in patent FR 2 679 771.

Examples that may also be mentioned include pigment pastes of organic pigments, such as the products sold by the company Hoechst under the names: Cosmenyl Yellow IOG: Yellow 3 pigment (CI 11710); Cosmenyl G yellow: Yellow 1 pigment (CI 11680); Cosmenyl GR orange: Orange 43 pigment (CI 71105); Cosmenyl R red: Red 4 pigment (CI 12085); Cosmenyl FB carmine: Red 5 pigment (CI 12490); Cosmenyl RL violet: Violet 23 pigment (CI 51319); Cosmenyl A2R blue: Blue 15.1 pigment (CI 74160); Cosmenyl GG green: Green 7 pigment (CI 74260); Cosmenyl R black: Black 7 pigment (CI 77266).

The pigments in accordance with the invention may also be in the form of composite pigments, as described in patent EP 1 184 426. These composite pigments may particularly be composed of particles including an inorganic core, at least one binder for attaching the organic pigments to the core, and at least one organic pigment which at least partially covers the core.

The organic pigment may also be a lake.

The term “lake” refers to dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate and aluminium. Among the dyes adsorbed on organic substrates, mention may be made of carminic acid. Mention may also be made of the dyes known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 4 (CI 15 510), D & C Red 33 (CI 17 200), D & C Yellow 5 (CI 19 140), D & C Yellow 6 (CI 15 985) D & C Green 5 (CI 61 570), D & C Yellow 10 (CI 77 002), D & C Green 3 (CI 42 053), D & C Blue 1 (CI 42 090), FDC Red 4, D & C Red 6, D & C Red 22, D & C Red 28, D & C Red 30, D & C Orange 4, D & C Yellow 8, D & C Green 5, D & C Red 17, D & C Green 6, D & C Yellow 11, D & C Violet 2, Sudan red, carotenes (β-carotene, lycopene), xanthophylls (capsanthin, capsorubin, lutein), palm oil, Sudan brown, quinoline yellow, annatto, curcumin, betanin (beet), carmine, copper chlorophyllin, methylene blue, anthocyanins (enocianine, black carrot, hibiscus, elderberry), caramel, riboflavin, beet juice and caramel.

An example of a lake that may be mentioned is the product known under the following name: D&C Red 7 (CI 15 850:1).

The pigment may also be a pigment with special effects.

The term “pigments with special effects” refers to pigments that generally create a coloured appearance (characterized by a certain shade, a certain vivacity and a certain level of luminance) that is non-uniform and that changes as a function of the conditions of observation (light, temperature, angles of observation, etc.). They thereby differ from coloured pigments, which afford a standard uniform opaque, semi-transparent or transparent shade.

Several types of special effect pigments exist: those with a low refractive index, such as fluorescent or photochromic pigments, and those with a higher refractive index, such as nacres, interference pigments or glitter flakes.

The size of the pigment used in the composition according to the present invention is generally between 10 nm and 200 μm, preferably between 20 nm and 80 μm and more preferentially between 30 nm and 50 μm.

The pigments may be dispersed in the composition by means of a dispersant.

This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium. In particular, esters of 12-hydroxystearic acid in particular and of C₈ to C₂₀ fatty acid and of polyols such as glycerol or diglycerol are used, such as poly(12-hydroxystearic acid) stearate with a molecular weight of approximately 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof. As other dispersants that may be used in the compositions of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C. The pigments used in the composition may be surface-treated with an organic agent. According to a particular embodiment, the dispersant(s) are of amino silicone type other than the alkoxysilanes described previously and are cationic. Preferably, the pigment(s) is (are) chosen from mineral, mixed mineral-organic, or organic pigments.

According to a particular embodiment, the pigment(s) according to the invention are organic pigments, preferentially organic pigments surface-treated with an organic agent chosen from silicone compounds.

According to another embodiment of the invention, the pigment(s) according to the invention are mineral pigments.

### Direct Dyes

According to a particular embodiment of the invention, the cosmetic active agent is a dyestuff chosen from one or more direct dyes.

The term “direct dye” means natural and/or synthetic dyes, other than oxidation dyes. These are dyes which will spread superficially over the fibre. They may be ionic or nonionic, preferably cationic or nonionic.

Among the direct dyes that are suitable for use in the invention, mention may be made of azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures.

The direct dyes are preferably cationic direct dyes. Mention may be made of the hydrazono cationic dyes of formulae (A) and (B) below and the azo cationic dyes of formulae (C) and (D) below:

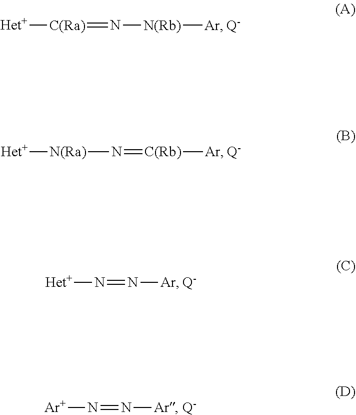

in which formulae (A) to (D):
Het⁺ represents a cationic heteroaryl radical, preferentially bearing an endocyclic cationic charge, such as imidazolium, indolium or pyridinium, which is optionally substituted, preferentially with at least one (C₁-C₈)alkyl group such as methyl;
Ar⁺ represents an aryl radical, such as phenyl or naphthyl, bearing an exocyclic cationic charge, preferentially ammonium, particularly tri(C₁-C₈)alkylammonium, such as trimethylammonium;
Ar represents an aryl group, notably phenyl, which is optionally substituted, preferentially with one or more electron-donating groups such as optionally substituted (C₁-C₈)alkyl, optionally substituted (C₁-C₈)alkoxy, (di)(C₁-C₈)(alkyl)amino optionally substituted on the alkyl group(s) with a hydroxyl group, aryl(C₁-C₈)alkylamino, and optionally substituted N—(C₁-C₈)alkyl-N-aryl(C₁-C₈)alkylamino or alternatively Ar represents a julolidine group;
Ar″ represents an optionally substituted (hetero)aryl group, such as phenyl or pyrazolyl, which are optionally substituted, preferentially with one or more (C₁-C₈)alkyl, hydroxyl, (di)(C₁-C₈)(alkyl)amino, (C₁-C₈)alkoxy or phenyl groups;
Ra and Rb, which may be identical or different, represent a hydrogen atom or a (C₁-C₈)alkyl group, which is optionally substituted, preferentially with a hydroxyl group; or else the substituent Ra with a substituent of Het⁺ and/or Rb with a substituent of Ar form, together with the atoms that bear them, a (hetero)cycloalkyl; in particular, Ra and Rb represent a hydrogen atom or a (C₁-C₄)alkyl group optionally substituted with a hydroxyl group;
Q′ represents an organic or mineral anionic counterion, such as a halide or an alkyl sulfate.

In particular, mention may be made of the azo and hydrazono direct dyes bearing an endocyclic cationic charge of formulae (A) to (D) as defined previously, more particularly the cationic direct dyes bearing an endocyclic cationic charge described in patent applications WO 95/15144, WO 95/01772 and EP 714 954.

Preferably, the direct dyes are chosen from the compounds of formulae (E) and (F) below:

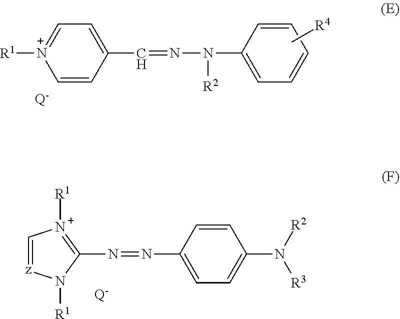

in which formulae (E) and (F):
R¹ represents a (C₁-C₄)alkyl group such as methyl;
R² and R³, which may be identical or different, represent a hydrogen atom or a (C₁-C₄)alkyl group, such as methyl;
R⁴ represents a hydrogen atom or an electron-donating group such as optionally substituted (C₁-C₈)alkyl, optionally substituted (C₁-C₈)alkoxy, or (di)(C₁-C₈) (alkyl)amino optionally substituted on the alkyl group(s) with a hydroxyl group; in particular, R₄ is a hydrogen atom;
Z represents a CH group or a nitrogen atom, preferentially CH;
Q⁻ is an anionic counterion as defined previously, in particular a halide, such as chloride, or an alkyl sulfate, such as methyl sulfate or mesyl.

In particular, the dyes of formulae (E) and (F) are chosen from Basic Red 51, Basic Yellow 87 and Basic Orange 31 or derivatives thereof with Q′ being an anionic counterion as defined previously, particularly a halide such as chloride, or an alkyl sulfate such as methyl sulfate or mesyl. The direct dyes may be chosen from anionic direct dyes. The anionic direct dyes of the invention are dyes commonly referred to as “acid” direct dyes owing to their affinity for alkaline substances.

The term “anionic direct dye” means any direct dye including in its structure at least one CO₂R′ or SO₃R′ substituent with R′ denoting a hydrogen atom or a cation originating from a metal or an amine, or an ammonium ion.

The anionic direct dyes may be chosen from direct nitro acid dyes, azo acid dyes, azine acid dyes, triarylmethane acid dyes, indoamine acid dyes, anthraquinone acid dyes, indigoid dyes and natural acid dyes.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions containing these natural dyes and particularly henna-based poultices or extracts.

Preferably, the direct dyes are chosen from anionic direct dyes.

The dyestuffs, preferably the pigments, may be present in concentrations ranging from 0.01% to 30% by weight, preferably from 0.02% to 20% by weight and more particularly from 0.05% to 15% by weight relative to the total weight of the composition containing them.

The direct dye(s) may be present in concentrations ranging from 0.001% to 10% by weight and preferably from 0.005% to 5% by weight relative to the total weight of the composition containing them.

Preferably, the cosmetic active agent(s), in particular the dyestuff(s) and more particularly the pigment(s), are introduced into at least one of the compositions “C1”, “C2”, “C3”, “C4” or “C5”.

### Care Active Agents

According to a particular embodiment, the process of the invention uses one or more care active agents.

More particularly, in the process of the invention, according to one embodiment, at least one of the compositions “C1”, “C2”, “C3”, “C4” or “C5” used comprises one or more care active agents, preferably in a proportion of at least 0.01% by weight relative to the total weight of the composition considered. In particular, the care active agent may be at least one hydrophilic active agent and/or one lipophilic active agent, and preferably a hydrophilic care active agent.

The term “hydrophilic active agent” means a water-soluble or water-dispersible active agent that is capable of forming hydrogen bonds.

The cosmetic care active agent(s) may notably be chosen from: a) vitamins and derivatives thereof, notably esters thereof, in particular tocopherol (vitamin E) and esters thereof (such as tocopheryl acetate), ascorbic acid (vitamin C) and derivatives thereof; b) humectants, in particular urea, hydroxyureas, glycerol, polyglycerols, glycerolglucoside, diglycerolglucoside, polyglycerylglucosides and xylitylglucoside, and in particular glycerol; c) C-glycoside compounds; d) antioxidant compounds; e) anti-ageing active agents, in particular hyaluronic acid compounds, and notably sodium hyaluronate, retinol and derivatives thereof, salicylic acid compounds and in particular n-octanoyl-5-salicylic acid (capryloyl salicylic acid), caffeine, adenosine, c-β-d-xylopyranoside-2-hydroxypropane and the sodium salt of 3-hydroxy-2-pentylcyclopentylacetic acid; f) skincare agents chosen from allantoin, panthenol and protein hydrolysates; g) polyphenols, notably escin, ruscus, diosmin, hesperidin and resveratrol, and h) and mixtures thereof.

In particular, in the process of the invention, according to one embodiment, at least one of the compositions “C1”, “C2”, “C3”, “C4” or “C5” used comprises a moisturizer (also known as a humectant agent). Preferably, the care active agent is a moisturizer, and in particular is glycerine (glycerol).

The care active agent(s) may in particular be present, in the composition containing same, in a content ranging from 0.01% to 30% by weight, relative to the weight of the composition, and preferably from 0.02% to 25% by weight.

### UV-Screening Agents

According to one embodiment of the process of the invention, at least one of the compositions “C1”, “C2”, “C3”, “C4” or “C5” used comprises, as cosmetic active agent, at least one UV-screening agent.

The UV-screening agent is a UV-screening agent normally used in cosmetics.

It may be chosen from the positive list contained in Annex VI of (EC) Regulation No. 1223/2009, which specifies the list of UV-screening agents permitted in cosmetics.

The UV-screening agents that are suitable for use in the invention may be of varied nature. They may be lipophilic, hydrophilic or insoluble organic agents.

The term “lipophilic UV-screening agent” means any cosmetic or dermatological screening agent that can be fully dissolved in molecular form in a liquid fatty phase or that can be dissolved in colloidal form (for example in micellar form) in a liquid fatty phase.

The term “hydrophilic UV-screening agent” means any cosmetic or dermatological screening agent that can be fully dissolved in molecular form in a liquid aqueous phase or that can be dissolved in colloidal form (for example in micellar form) in a liquid aqueous phase.

The term “insoluble UV-screening agent” means any cosmetic or dermatological screening agent which is not defined either as a lipophilic UV-screening agent or as a hydrophilic UV-screening agent, and which is in the form of particles in aqueous phase or liquid fatty phase.

The UV-screening agents of the composition according to the invention may afford UVA and/or UVB photoprotection.

According to a preferred embodiment, the compositions, which are preferably cosmetic compositions, may comprise at least one organic and/or mineral UV-screening agent (for screening out the UV radiation of sunlight).

In particular, the UV-screening agent(s) are chosen from bis-resorcinyl triazine derivatives, dibenzoylmethane derivatives, benzylidene camphor derivatives, and mixtures thereof. The organic UV-screening agents may also be chosen from anthranilic derivatives; cinnamic derivatives; salicylic derivatives; benzophenone derivatives; phenylbenzotriazole derivatives; benzalmalonate derivatives, notably those mentioned in patent U.S. Pat. No. 5,624,663; phenylbenzimidazole derivatives; imidazolines; 4,4-diarylbutadiene derivatives; bis-benzazolyl derivatives, as described in patents EP 6 693 23 and U.S. Pat. No. 2,463,264; p-aminobenzoic acid (PABA) derivatives; methylenebis(hydroxyphenylbenzotriazole) derivatives, as described in patent applications U.S. Pat. Nos. 5,237,071, 5,166,355, GB 2 303 549, DE 197 26, 184 and EP 893 119; benzoxazole derivatives, such as those described in patent applications EP 0 832 642, EP 1 027 883, EP 1 300 137 and DE 101 62 844; screening polymers and screening silicones such as those notably described in patent application WO 93/04665; α-alkylstyrene-based dimers such as those described in patent application DE 198 55 649; 4,4-diarylbutadienes such as those described in patent applications EP 0 967 200, DE 197 46 654, DE 197 55 649, EP 1 008 586, EP 1 133 980 and EP 133 981; other merocyanine derivatives such as those described in patent applications WO 04/006878, WO 05/058269 and WO 06/032741, and mixtures thereof.

According to a particular embodiment, the concentration of the organic UV-screening agents in the compositions ranges from 1% to 50%, preferably from 1% to 40% by weight, for example from 5% to 35% by weight, relative to the total weight of the composition.

The UV-screening agent(s) may be mineral UV-screening agents, which are generally pigments. The pigments may or may not be coated.

Thus, the mineral UV-screening agents may be chosen from coated or uncoated pigments, and in particular from coated titanium oxide pigments, silicone-treated titanium oxides, uncoated titanium oxide pigments, uncoated zinc oxide pigments, coated zinc oxide pigments, uncoated cerium oxide pigments, uncoated iron oxide pigments, coated iron oxide pigments, and mixtures thereof.

According to a particular embodiment, compositions “C1” to “C5” are free of mineral UV-screening agents.

According to a particular embodiment, the amount of the mineral UV-screening agent(s) present in compositions “C1”, “C2”, “C3”, “C4” or “C5” according to the invention may range from 0.01% to 20% by weight relative to the total weight of the composition containing same. It ranges, for example, from 1% to 15% by weight, relative to the total weight of the composition.

According to a particular embodiment, at least one of the compositions “C1” to “C5” also comprises one or more organic UV-screening agents and one or more mineral UV-screening agents.

According to a particular embodiment, the compositions comprise a combination of UV-screening agents as described in patent FR 2 977 490, patent application WO 2013/004777 or patent application US 2014/0134120.

Preferably, the process for treating keratin materials, notably the skin and/or the lips and/or the eyelashes and/or the eyebrows, and compositions “C1”, “C2”, “C3”, “C4” or “C5” use or comprise one or more dyestuffs chosen from pigments, direct dyes and mixtures thereof, preferably pigments; more preferentially, the pigment(s) of the invention are chosen from carbon black, iron oxides, notably yellow, red and black iron oxides, and micas coated with iron oxide, triarylmethane pigments, notably blue and violet triarylmethane pigments, such as Blue 1 Lake, azo pigments, notably red azo pigments, such as D&C Red 7, an alkali metal salt of lithol red, such as the calcium salt of lithol red B, even more preferentially red iron oxides, yellow iron oxides and azo pigments, notably red azo pigments, such as D&C Red 7.

### Fatty Phase—Fatty Substances

According to a particular embodiment, the process of the invention also uses iv) one or more fatty substances, in particular one or more oils, preferably volatile oils.

In particular, at least one of the compositions “C1”, “C2”, “C3”, “C4” or “C5” used in the process of the invention contains a fatty phase.

In particular, at least one of the compositions “C1”, “C2”, “C3”, “C4” or “C5” used in the process of the invention comprises one or more fatty substances, different from the compounds i), in particular one or more oils, preferably volatile oils.

The term “oil” refers to a fatty substance that is liquid at room temperature (20° C.) and atmospheric pressure (760 mmHg).

The term “hydrocarbon-based oil” means an oil formed essentially from, or even constituted of, carbon and hydrogen atoms, and possibly oxygen and nitrogen atoms, and not containing any silicon or fluorine atoms. It may contain alcohol, ester, ether, carboxylic acid, amine and/or amide groups.

According to one embodiment of the invention, the oil(s) iv) are chosen from volatile oils, in particular:
hydrocarbon-based oils containing from 8 to 16 carbon atoms, in particular branched C₈-C₁₆ alkanes, in particular isoalkanes, more particularly isoalkanes (also known as isoparaffins), preferably C₁₃-C₁₆ isoparaffins, isododecane, isodecane, isohexadecane, for example the oils sold under the trade names Isopar or Permethyl, alone or as mixtures, preferably isododecane (also known as 2,2,4,4,6-pentamethylheptane), linear alkanes, in particular C₁₁-C₁₆ alkanes, alone or as mixtures, in particular hexane, decane, undecane, tridecane, isoparaffins, in particular n-dodecane (C₁₂) and n-tetradecane (C₁₄), the undecane-tridecane mixture, mixtures of n-undecane (C₁₁) and n-tridecane (C₁₃), and mixtures thereof and also mixtures of n-undecane (C₁₁) and n-tridecane (C₁₃), and volatile C₅-C₁₂ cyclic, non-aromatic alkanes;
short-chain esters containing from 3 to 8 carbon atoms in total, in particular ethyl acetate, methyl acetate, propyl acetate or n-butyl acetate;
carbonate hydrocarbon-based oils of structure R′₁—O—C(O)—O—R′₂ in which R′₁ and R′₂ independently denote a linear, branched or cyclic C₄-C₈ alkyl group, preferably a C₄-C₈ alkyl group, advantageously chosen more preferentially from dibutyl carbonate or dipentyl carbonate;
ether oils of formula R₁—O—R₂ in which R₁ and R₂, independently of each other, denote a linear, branched or cyclic C₄-C₈ alkyl group, preferably a C₄-C₈ alkyl group;
silicone oils, in particular comprising from 2 to 7 silicon atoms, and optionally including alkyl or alkoxy groups containing from 1 to 10 carbon atoms, in particular dimethicones of viscosity 5 and 6 cSt, cyclopentadimethylsiloxane, dodecamethylpentasiloxane, cyclohexadimethylsiloxane, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof;
   more preferentially, the volatile oil(s) iv) are chosen from C₈-C₁₆ alkanes, in particular branched alkanes, preferably isododecane.

In particular, at least one of the compositions “C1”, “C2”, “C3”, “C4” or “C5” used in the process according to the invention comprises one or more non-volatile oils, preferably chosen from:
non-volatile fluoro oils, in particular chosen from fluorinated polyethers, fluorosilicone oils and fluorosilicones;
non-volatile silicone oils, in particular chosen from non-volatile silicones having the following INCI names: dimethicone, dimethiconol, trimethyl pentaphenyl trisiloxane, tetramethyl tetraphenyl trisiloxane, diphenyl dimethicone, trimethylsiloxyphenyl dimethicone, phenyl trimethicone, diphenylsiloxy phenyl trimethicone; and also mixtures thereof;
non-volatile apolar hydrocarbon-based oils, in particular chosen from linear or branched compounds of mineral or synthetic origin: i) liquid paraffin, ii) squalane, isoeicosane, iii) mixtures of linear, saturated hydrocarbons, more particularly C₁₅-C₂₈ hydrocarbons, in particular mixtures whose INCI names are (C₁₅-C₁₉)Alkane, (C₁₈-C₂₁)Alkane, (C₂₁-C₂₈)Alkane, iv) hydrogenated or non-hydrogenated polybutenes; v) hydrogenated or non-hydrogenated polyisobutenes, preferably hydrogenated, vi) hydrogenated or non-hydrogenated polydecenes, vii) decene/butene copolymers, butene/isobutene copolymers and viii) mixtures thereof;
non-volatile polar hydrocarbon-based oils, which may be chosen from:
i) saturated, unsaturated, linear or branched C₁₀-C₂₆ fatty alcohols, preferably monoalcohols; in particular, the C₁₀-C₂₆ alcohols are fatty alcohols, which are preferably branched when they comprise at least 16 carbon atoms; preferably, the fatty alcohol comprises from 10 to 24 carbon atoms, and more preferentially from 12 to 22 carbon atoms, in particular such as lauryl alcohol, isostearyl alcohol, oleyl alcohol, 2-butyloctanol, 2-undecylpentadecanol, 2-hexyldecyl alcohol, isocetyl alcohol, octyldodecanol and mixtures thereof;
ii) triglycerides consisting of fatty acid esters of glycerol, in particular the fatty acids of which may have chain lengths ranging from C₄ to C₃₆, and notably from C₁₈ to C₃₆, these oils possibly being linear or branched, and saturated or unsaturated; by way of example, mention may notably be made of heptanoic or octanoic triglycerides, caprylic/capric acid triglycerides, plant oils such as wheatgerm oil, sunflower oil, grapeseed oil, sesame seed oil, corn oil, apricot kernel oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil, musk rose oil, groundnut oil, coconut oil, argan oil, passionflower oil, kaya oil; the liquid fraction of shea butter, and the liquid fraction of cocoa butter; and also mixtures thereof;
iii) linear aliphatic hydrocarbon-based esters of formula R—C(O)—OR′ in which R—C(O)—O— represents the carboxylic acid residue containing from 2 to 40 carbon atoms, and R′ represents a hydrocarbon-based chain containing from 1 to 40 carbon atoms, aliphatic hydrocarbon-based esters of alkylene glycol, in particular ethylene glycol or propylene glycol; the total number of carbon atoms in particular being at least 10; notably chosen from isoamyl laurate, cetostearyl octanoate, isopropyl myristate, isopropyl palmitate, isopropyl stearate or isostearate, ethyl palmitate, 2-ethylhexyl palmitate, isostearyl isostearate, octyl stearate, isostearyl heptanoate, octanoates, decanoates or ricinoleates of alcohols or of polyalcohols, such as propylene glycol dioctanoate, cetyl octanoate or tridecyl octanoate, 2-ethylhexyl palmitate, alkyl benzoate, polyethylene glycol diheptanoate, propylene glycol bis(2-ethylhexanoate) and mixtures thereof, hexyl laurate, neopentanoic acid esters, such as isodecyl neopentanoate, isotridecyl neopentanoate, isostearyl neopentanoate or 2-octyldodecyl neopentanoate, isononanoic acid esters, in particular isononyl isononanoate, isotridecyl isononanoate or octyl isononanoate, oleyl erucate, isopropyl lauroyl sarcosinate, diisopropyl sebacate, isocetyl stearate, isodecyl neopentanoate, isostearyl behenate or myristyl myristate;
iv) hydroxylated esters, in particular polyglyceryl-2 triisostearate;
v) aromatic esters, in particular tridecyl trimellitate, C₁₂-C₁₅ alcohol benzoate, the 2-phenylethyl ester of benzoic acid, and butyloctyl salicylate;
vi) linear fatty acid esters with a total carbon number ranging from 35 to 70, in particular pentaerythrityl tetrapelargonate;
vii) esters of C₂₄-C₂₈ branched fatty acids or fatty alcohols, in particular triisoarachidyl citrate, pentaerythrityl tetraisononanoate, glyceryl triisostearate, glyceryl tris(2-decyltetradecanoate), pentaerythrityl tetraisostearate, polyglyceryl-2 tetraisostearate or pentaerythrityl tetrakis(2-decyltetradecanoate);
viii) the polyesters obtained by condensation of dimer and/or trimer of unsaturated fatty acid and of diol, in particular those with the INCI name Dilinoleic Acid/Butanediol Copolymer or Dilinoleic Acid/Propanediol Copolymer; the polyesters obtained by condensation of fatty acid dimer and of diol dimer, in particular dimer dilinoleyl dimer dilinoleate;
ix) synthetic ethers containing from 10 to 40 carbon atoms, in particular dicaprylyl ether;
x) dialkyl carbonates, the two alkyl chains possibly being identical or different, in particular dicaprylyl carbonate;
xi) vinylpyrrolidone copolymers, in particular vinylpyrrolidone/1-hexadecene copolymer; and
xii) mixtures thereof;
the non-volatile carbonate oils may be chosen from the carbonates of formula R₈—O—C(O)—O—R₉, with R₈ and R₉, which may be identical or different, representing a linear or branched C₄ to C₁₂ and preferentially C₆ to C₁₀ alkyl chain; the carbonate oils may be dicaprylyl carbonate (or dioctyl carbonate), bis(2-ethylhexyl)carbonate, dipropylheptyl carbonate, dibutyl carbonate, dineopentyl carbonate, dipentyl carbonate, dineoheptyl carbonate, diheptyl carbonate, diisononyl carbonate or dinonyl carbonate and preferably dioctyl carbonate;
oils known as non-volatile ether oils of formula R₁—O—R₂ in which R₁ and R₂ independently denote a linear, branched or cyclic C₆-C₂₄ alkyl group, preferably a C₆-C₁₈ alkyl group, and preferably C₈-C₁₂ alkyl group. It may be preferable for R₁ and R₂ to be identical. Linear alkyl groups that may be mentioned include a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group, a behenyl group, a docosyl group, a tricosyl group and a tetracosyl group. Branched alkyl groups that may be mentioned include a 1,1-dimethylpropyl group, a 3-methylhexyl group, a 5-methylhexyl group, an ethylhexyl group, a 2-ethylhexyl group, a 5-methyloctyl group, a 1-ethylhexyl group, a 1-butylpentyl group, a 2-butyloctyl group, an isotridecyl group, a 2-pentylnonyl group, a 2-hexyldecyl group, an isostearyl group, a 2-heptylundecyl group, a 2-octyldodecyl group, a 1,3-dimethylbutyl group, a 1-(1-methylethyl)-2-methylpropyl group, a 1,1,3,3-tetramethylbutyl group, a 3,5,5-trimethylhexyl group, a 1-(2-methylpropyl)-3-methylbutyl group, a 3,7-dimethyloctyl group and a 2-(1,3,3-trimethylbutyl)-5,7,7-trimethyloctyl group. As cyclic alkyl groups, mention may be made of a cyclohexyl group, a 3-methylcyclohexyl group and a 3,3,5-trimethylcyclohexyl group, dilauryl ether, diisostearyl ether, dioctyl ether, nonylphenyl ether, dodecyl dimethylbutyl ether, cetyl dimethylbutyl ether and mixtures thereof.

Preferably, the non-volatile oil(s) are chosen from hydrogenated or non-hydrogenated polyisobutenes, preferably hydrogenated, in particular the non-volatile compounds of the Parleam® range; mixtures of C₁₅-C₁₉ alkanes, and from linear aliphatic hydrocarbon-based esters of formula R—C(O)—OR′ in which R—C(O)—O represents a carboxylic acid residue containing from 2 to 40 carbon atoms, and R′ represents a hydrocarbon-based chain containing from 1 to 40 carbon atoms, as defined previously, in particular isononyl isononanoate.

More preferentially, the process of the invention uses one or more hydrocarbon-based oils containing from 8 to 16 carbon atoms, and notably branched C₈-C₁₆ alkanes, in particular isoalkanes, preferably C₁₃-C₁₆ isoparaffins, isododecane, isodecane, isohexadecane, alone or as mixtures, and more preferentially isododecane.

According to a preferred embodiment, the process of the invention uses one or more fatty substances iv), different from the compounds of formula (I), in particular at least one oil, preferably at least one volatile oil, more preferentially at least one hydrocarbon-based volatile oil.

In particular, the amount of oil(s), different from the compounds i), in at least one of the compositions “C1”, “C2”, “C3”, “C4” or “C5” used in the process according to the invention ranges from 1% to 99% by weight, relative to the total weight of the composition, more particularly from 2% to 98% by weight, preferentially from 3% to 97%, better still from 5% to 96% by weight, relative to the total weight of the composition.

According to another particular embodiment, the process of the invention does not use any fatty substances iv) different from the compounds of formula (I).

According to yet another embodiment, at least one of the compositions “C1”, “C2”, “C3”, “C4” or “C5” used in the process according to the invention contains a mixture of fatty substances v) different from the compounds i), and chosen from mixtures of at least one volatile oil, preferably a hydrocarbon-based oil, and at least one non-volatile oil, such as octyldodecanol.

### Compositions

Another object of the invention is compositions, in particular cosmetic compositions, containing at least one compound chosen from the compounds of formulae (Ia), (Ib), (Ic), (Id) and (Ie), as described previously, and also the salts, isomers and/or solvates thereof, such as hydrates, more particularly compositions, notably cosmetic compositions, containing at least one compound of formulae (Iic), (Iid) and (Iie), as described previously.

Another subject of the invention is compositions, notably cosmetic compositions, containing at least one compound chosen from compounds (8), (9), (10), (11), (12), (13) and (14), as described previously, and also the salts, isomers and solvates thereof.

Another subject of the invention is compositions, in particular cosmetic compositions, containing at least one compound chosen from the compounds of formulae (I), (Ia), (Ib), (Ic), (Id) and/or (Ie), as described previously, and also the salts, isomers and/or solvates thereof, such as hydrates, other than the compounds (1) to (7) as defined previously.

According to a particular embodiment, the process for treating keratin materials, notably the skin and/or the lips and/or the eyelashes and/or the eyebrows, according to the invention uses water. Thus, according to this variant, at least one of the compositions “C1” to “C5” used in the process of the invention comprises water. According to this variant, the compositions “C1”, “C2”, “C3”, “C4” and/or “C5” are aqueous or aqueous-alcoholic compositions.

According to a preferred embodiment, the process for treating keratin materials, notably the skin and/or the lips and/or the eyelashes and/or the eyebrows, according to the invention does not use any water. Thus, according to this variant, the compositions “C1”, “C2”, “C3”, “C4” and/or “C5” are anhydrous compositions.

According to a particular embodiment, at least one of the compositions “C1”, “C2”, “C3”, “C4” and/or “C5” comprises an isododecane/ethanol mixture, in particular in a volume ratio ranging from 1/99 to 99/1, more particularly from 5/95 to 95/5.

According to a particular embodiment, at least one of the compositions “C1”, “C2”, “C3”, “C4” and/or “C5” comprises an isododecane/octyldodecanol mixture, in particular in a volume ratio ranging from 1/99 to 99/1, more particularly from 5/95 to 95/5.

According to a particular embodiment, at least one of the compositions “C1”, “C2”, “C3”, “C4” or “C5” may be in the form of a water-in-oil emulsion or an oil-in-water emulsion.

According to a particular embodiment, the compositions “C1”, “C4” or “C5” may be in anhydrous, water-in-oil emulsion or oil-in-water emulsion form.

According to a particular embodiment, composition “C4” is anhydrous. In particular, composition “C4” is anhydrous, and it comprises at least one oil, in particular a volatile oil, preferably isododecane.

According to another particular embodiment, composition “C4” comprises water. In particular, composition “C4” comprises water, and optionally at least one volatile or non-volatile oil, and/or at least one C1-C4 alcohol such as ethanol or isopropanol, and/or at least one non-volatile oil.

The compositions “C1” to “C5” used in the process of the invention may also comprise one or more solvents, other than water.

According to a particular embodiment of the invention, the compound(s) of formula (I) as defined previously are used in a medium containing at least one solvent, preferably at least one polar and/or protic solvent, other than water.

According to this embodiment, composition “C1”, “C2”, “C3”, “C4” and/or “C5” used in the process of the invention comprises one or more solvents, preferably one or more polar and/or protic solvents other than water.

Preferably, composition “C1”, “C2”, “C3”, “C4” and/or “C5” used in the process of the invention comprises at least one polar and/or protic solvent chosen from monoalcohols containing from 2 to 6 carbon atoms, in particular chosen from ethanol, propanol, n-butanol, isopropanol, isobutanol, tert-butanol, pentanol and hexanol, preferably n-butanol or ethanol, and even more preferentially ethanol.

According to one embodiment, composition “C1”, “C2”, “C3”, “C4” and/or “C5” used in the process of the invention comprises one or more solvents other than water, in a content of less than 95% by weight, and more preferentially less than 92% by weight, relative to the total weight of the composition.

Organic solvents that may also be mentioned include polyols that are miscible with water at room temperature (25° C.), notably chosen from polyols notably containing from 2 to 10 carbon atoms, preferably containing from 2 to 6 carbon atoms, such as glycerol, propylene glycol, 1,3-propanediol, butylene glycol, pentylene glycol, hexylene glycol, dipropylene glycol, diethylene glycol or diglycerol; polyol ethers, such as 2-butoxyethanol, propylene glycol monomethyl ether, diethylene glycol monoethyl ether or diethylene glycol monomethyl ether; and also aromatic alcohols, such as benzyl alcohol, and mixtures thereof.

According to a particular embodiment, the composition also comprises one or more polyols notably chosen from polyols notably containing from 2 to 10 carbon atoms, preferably containing from 2 to 6 carbon atoms, preferably glycerol.

The compositions “C1”, “C2”, “C3”, “C4” or “C5” used in the process of the invention may also comprise one or more adjuvants commonly used in cosmetics, in particular chosen from thickeners, film-forming agents, gelling agents, trace elements, softeners, sequestrants, fragrances, basifying or acidifying agents, dispersants, preserving agents, fillers, surfactants, propellants, polar additives and polymers, or mixtures thereof.

Another subject of the invention is a composition “C3” comprising i) at least one compound of formula (I), or (Ia), (Ib), (Ic), (Id), (Ie), (Iic), (Iid) and/or (Iie), as defined previously, ii) at least one crosslinking agent, in particular as defined previously, and iii) at least one cosmetic active agent, in particular as defined previously.

Another subject of the invention is the cosmetic use of composition “C3” as defined previously, for treating, for caring for and/or making up keratin materials, notably the skin and/or the lips and/or the eyelashes and/or the eyebrows.

A composition “C1”, “C2”, “C3”, “C4” or “C5” is generally suitable for application to keratin materials, in particular application to the skin and/or the lips, and thus generally comprises a physiologically acceptable medium, i.e. a medium that is compatible with keratin materials, in particular application to the skin and/or the lips.

It is preferably a cosmetically acceptable medium, i.e. a medium which has a pleasant colour, odour and feel and which does not cause any unacceptable discomfort, i.e. stinging or tautness, liable to discourage the user from applying this composition.

A composition “C1”, “C2”, “C3”, “C4” or “C5” may be in the form of a makeup product, in particular a coloured makeup product, for the skin, in particular a foundation, optionally having care properties, a blusher, a face powder, an eyeshadow, a concealer, an eyeliner; a lip makeup product such as a lipstick, optionally having care properties, a lip gloss or lip pencils; a makeup product for the integuments such as the nails or eyelashes, in particular in the form of a cake mascara, or for the eyebrows, or a product for the temporary tattooing of bodily skin.

According to a particular embodiment, a composition “C1”, “C2”, “C3”, “C4” or “C5” is in the form of:
either a coloured product for the lips; or
a skincare product, which may be coloured, in particular a cream or a fluid having moisturizing and/or filling and/or tensor properties.

### Kit

According to yet another of its aspects, the present invention is also directed towards a multi-compartment kit or device, notably a cosmetic kit or device, comprising:
at least one compartment containing i) at least one compound of formula (I), or (Ia), (Ib), (Ic), (Id) and/or (Ie) as defined previously, and optionally iii) at least one cosmetic active agent, in particular as defined previously, in particular comprising composition “C1” as defined previously;
at least one compartment different from the one which contains i) and containing ii) at least one crosslinking agent, in particular as defined previously, and optionally iii) at least one cosmetic active agent, in particular as defined previously, in particular comprising composition “C4” as defined previously; and
optionally, at least one compartment different from those containing i) and ii), and containing iii) at least one cosmetic active agent, in particular as defined previously, which may be identical to or different from that/those optionally contained in the compartments comprising i) and ii).

Preferably, the compartment comprising at least i), in particular a composition “C1”, does not comprise any crosslinking agent ii).

Preferably, the compartment comprising at least one crosslinking agent ii), in particular a composition “C4”, does not contain i) any compound of formula (I).

The invention is illustrated in greater detail by the examples presented below. Unless otherwise indicated, the amounts indicated are expressed as mass percentages.

### Example

### Methods and Measurements

### Skin Application

### a) One-Step Protocol

The compounds of formula (I) bearing acetoacetate functions, and the crosslinking agents are mixed together before application. The system remains fluid long enough to allow application to the substrate.

The compositions are applied to a Bioskin type vitro support from Maprecos (Bioskin plate #10) (elastomeric skin-simulating support) using a film spreader (wet thickness of 100 μm).

The deposit is left to dry for 24 hours.

After 24 hours of drying, the deposits are evaluated according to the protocols described hereinbelow.

### b) Two-Step Protocol

A first composition, termed the “base coat”, is applied to a Bioskin type vitro support from Maprecos (Bioskin plate #10) (elastomeric skin-simulating support) using a film spreader (wet thickness of 100 μm). The deposit is left to dry for 24 hours.

After this first gesture, a second composition, termed the “top coat”, is applied in the same way.

The deposit is left to dry for 24 hours.

After 24 hours of drying, the deposits are evaluated according to the protocols described hereinbelow.

### Resistance to Adhesive Tape

A piece of adhesive tape (Scotch® Magic™ 810 from 3M; w=19 mm, L=5 cm) is applied to the deposit. A weight of approximately 1070 g is placed on the piece of adhesive tape for 30 seconds. The piece of adhesive tape is then removed and mounted on a slide holder so as to properly observe the result. The adhesion of the film to the support is thus evaluated.

### Resistance to Olive Oil/Sebum/Water

0.5 mL of olive oil, sebum or water is applied to the film of formulation. After 5 minutes, the olive oil, sebum or water is removed by wiping 15 times with cotton wool. The deterioration of the film following contact with the drop of attacking agents is thus examined.

The resistance is evaluated according to the following scale:
+++: no attack of the deposit, which is as it was originally;
++: a little transfer but the deposit is as it was originally;
+: the deposit is slightly altered and some transfer is observed;
0: the deposit is completely degraded and there is a lot of transfer to the cotton wool.

### Fragmentation Test of Formulations on Bioskin

As for the tests of resistance to olive oil/sebum/water and to adhesive tape, films are applied to a sample of Bioskin. After drying for one day, the Bioskin plate is stretched ten times using manual force.

Next, the result is observed on the film (fragmentation or no fragmentation).

The evaluation is carried out as follows:
+++: Cosmetic property evaluated as highly effective;
++: Cosmetic property evaluated as moderately effective;
+: Cosmetic property evaluated as somewhat effective;
0: Cosmetic property evaluated as ineffective.

### Example 1: Synthesis of Castor Oil Bearing an Acetoacetate Function (Compound 1)

The synthesis is represented by the following preparation scheme:

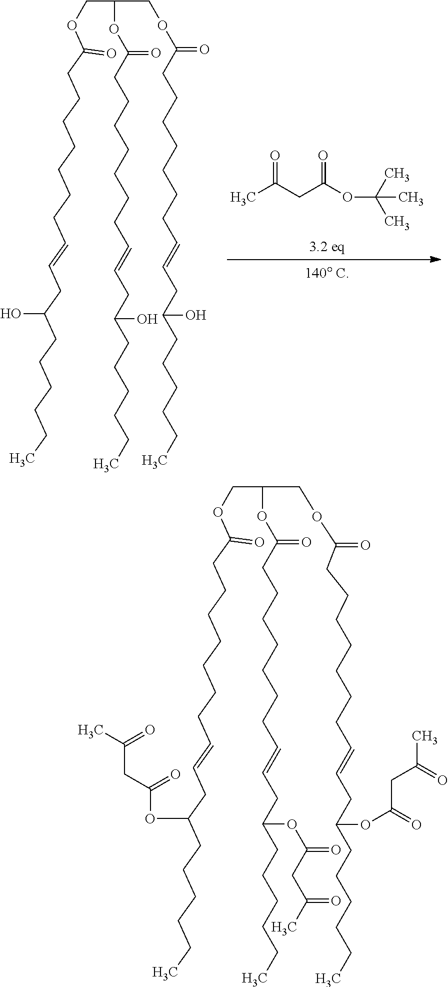 [SMILES: CCCCCCC(O)C/C=C/CCCCCCCC(=O)OCC(COC(=O)CCCCCCC/C=C/CC(O)CCCCCC)OC(=O)CCCCCCC/C=C/CC(O)CCCCCC ; CC(=O)CC(=O)OC(C)(C)C ; CCCCCCC(C/C=C/CCCCCCCC(=O)OCC(COC(=O)CCCCCCC/C=C/CC(CCCCCC)OC(=O)CC(C)=O)OC(=O)CCCCCCC/C=C/CC(CCCCCC)OC(=O)CC(C)=O)OC(=O)CC(C)=O]

50 g of castor oil (sold by the company Interchimie) and 25.40 g of tert-butyl acetoacetate are placed in a 250 mL SVL® reactor equipped with a mechanical stirrer, a thermometer and a distillation column. The reaction medium is heated at 130° C. on an oil bath. After 7 hours, the reaction medium is cooled, placed in a 250 ml pear-shaped flask and heated at 120° C. under continuous vacuum on a rotavapor for 1 hour.

### Example 2: Synthesis of Hydroxylated Castor Oil Bearing an Acetoacetate Function (Compound 2)

The synthesis is represented by the following preparation scheme:

### Step a):

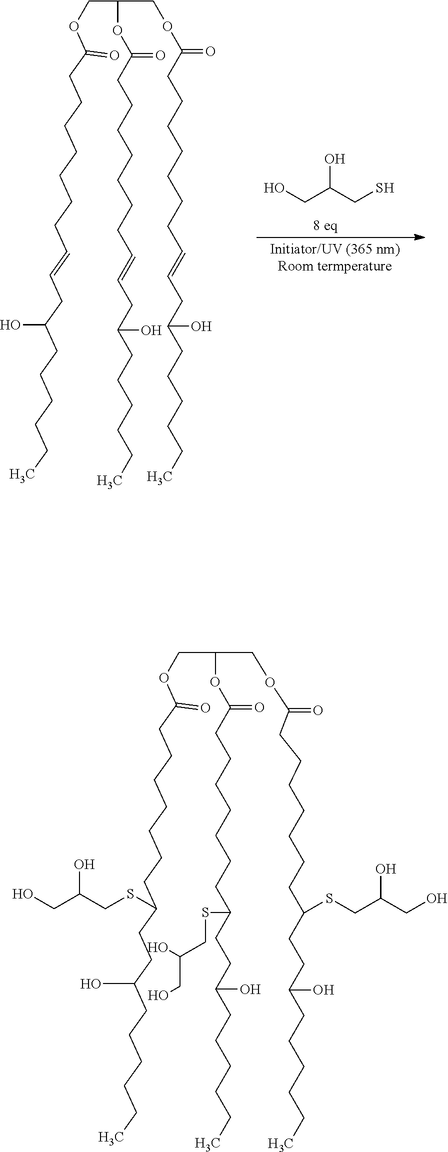 [SMILES: CCCCCCC(O)C/C=C/CCCCCCCC(=O)OCC(COC(=O)CCCCCCC/C=C/CC(O)CCCCCC)OC(=O)CCCCCCC/C=C/CC(O)CCCCCC ; OCC(O)CS ; CCCCCCC(O)CCC(CCCCCCCC(=O)OCC(COC(=O)CCCCCCCC(CCC(O)CCCCCC)SCC(O)CO)OC(=O)CCCCCCCC(CCC(O)CCCCCC)SCC(O)CO)SCC(O)CO]

### Step b):

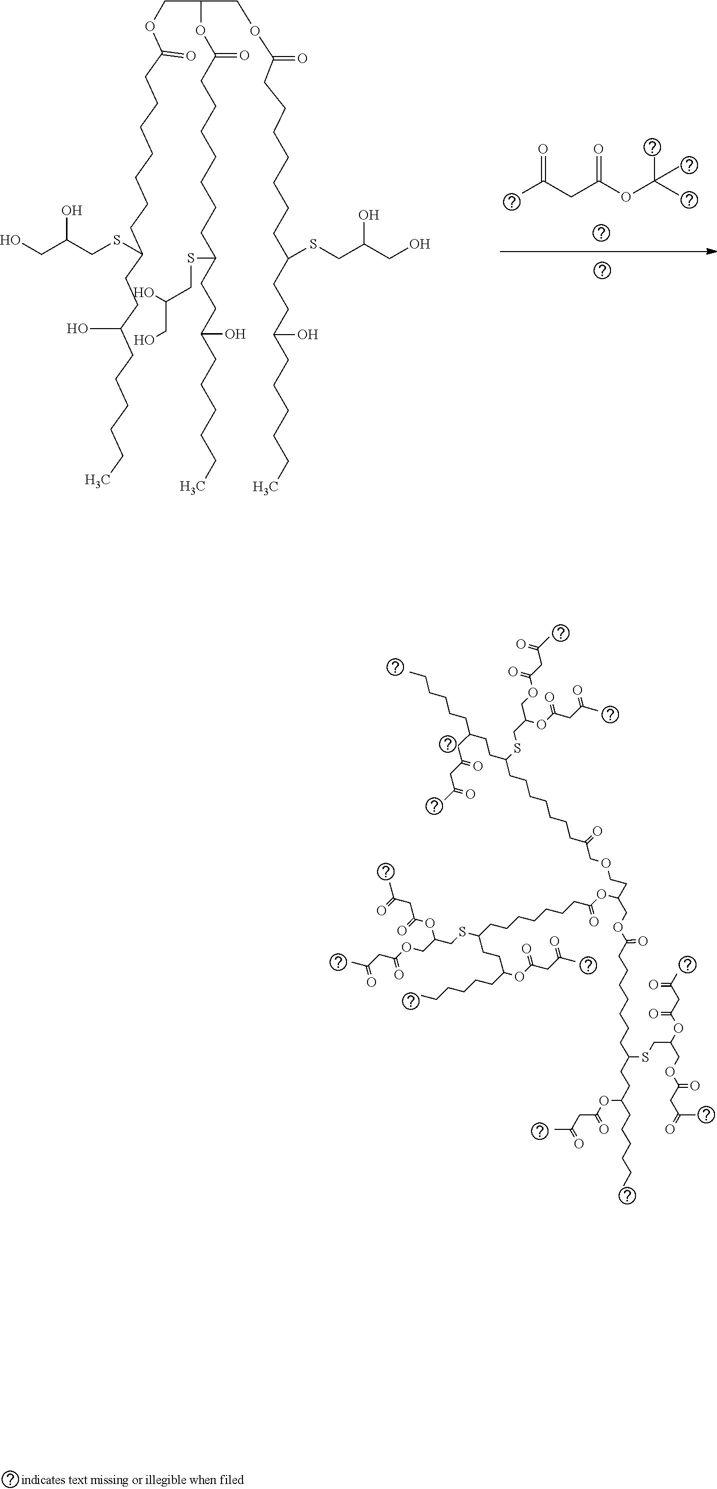 [SMILES: CCCCCCC(O)CCC(CCCCCCCC(=O)OCC(COC(=O)CCCCCCCC(CCC(O)CCCCCC)SCC(O)CO)OC(=O)CCCCCCCC(CCC(O)CCCCCC)SCC(O)CO)SCC(O)CO ; CC(=O)CC(=O)OC(C)(C)C ; CCCCCCC(CCC(CCCCCCCC(=O)COCCC(COC(=O)CCCCCCCC(CCC(CCCCCC)OC(=O)CC(C)=O)SCC(COC(=O)CC(C)=O)OC(=O)CC(C)=O)OC(=O)CCCCCCCC(CCC(CCCCCC)OC(=O)CC(C)=O)SCC(COC(=O)CC(C)=O)OC(=O)CC(C)=O)SCC(COC(=O)CC(C)=O)OC(=O)CC(C)=O)CC(=O)CC(C)=O]

In step a), the hydroxylated castor oil is prepared: 10 g of castor oil (sold by the company Interchimie), 9 g of 3-mercapto-1,2-propanediol (sold by the company Sigma-Aldrich) and 0.7 g of 2-hydroxy-2-methylpropiophenone (sold by the company Sigma-Aldrich) are added to 20 mL of ethyl acetate in a 250 mL round-bottomed flask equipped with a magnetic stirrer, and stirred for 5 minutes under argon. The reaction medium is then irradiated under a UV lamp (100 W, 365 nm) for 4 hours, and then washed three times with NaCl-saturated water. The organic phase is separated out, dried over MgSO₄ and then concentrated under vacuum. Finally, the solvent and the traces of thiol and of photoinitiator (2-hydroxy-2-methylpropiophenone) are removed by distillation under reduced pressure. The product obtained is used as is in the next step.

In step b), the hydroxylated castor oil is functionalized with acetoacetate groups: 10 g of hydroxylated castor oil obtained in step a) and 12 g of tert-butyl acetoacetate are placed in a 250 ml round-bottomed flask equipped with a magnetic stirrer. The reaction medium is heated at 130° C. on an oil bath. After 4 hours, the reaction medium is placed under continuous vacuum for 1 hour. A yellow viscous oil is obtained.

### Example 3: Synthesis of Epoxidized Oil Bearing an Acetoacetate Function (Compound 3)

The synthesis is represented by the following preparation scheme:

### Step a):

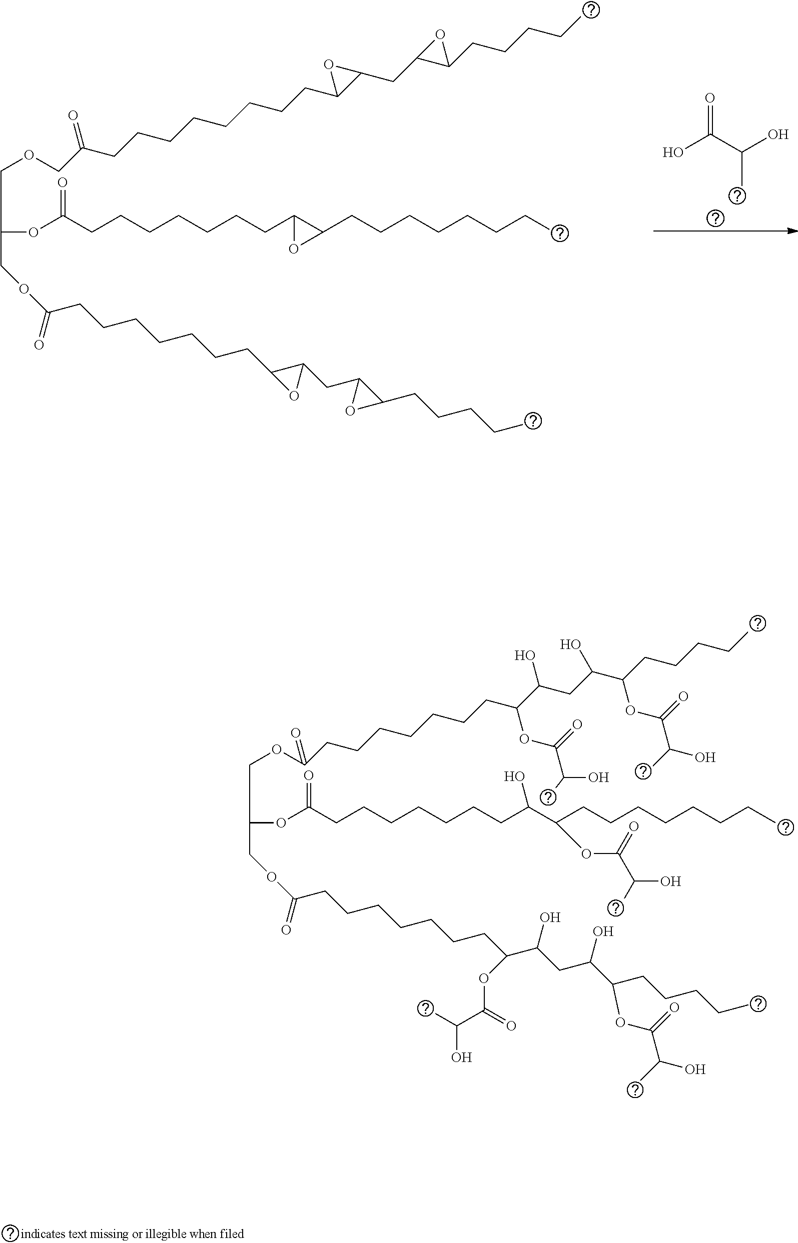

### Step b):

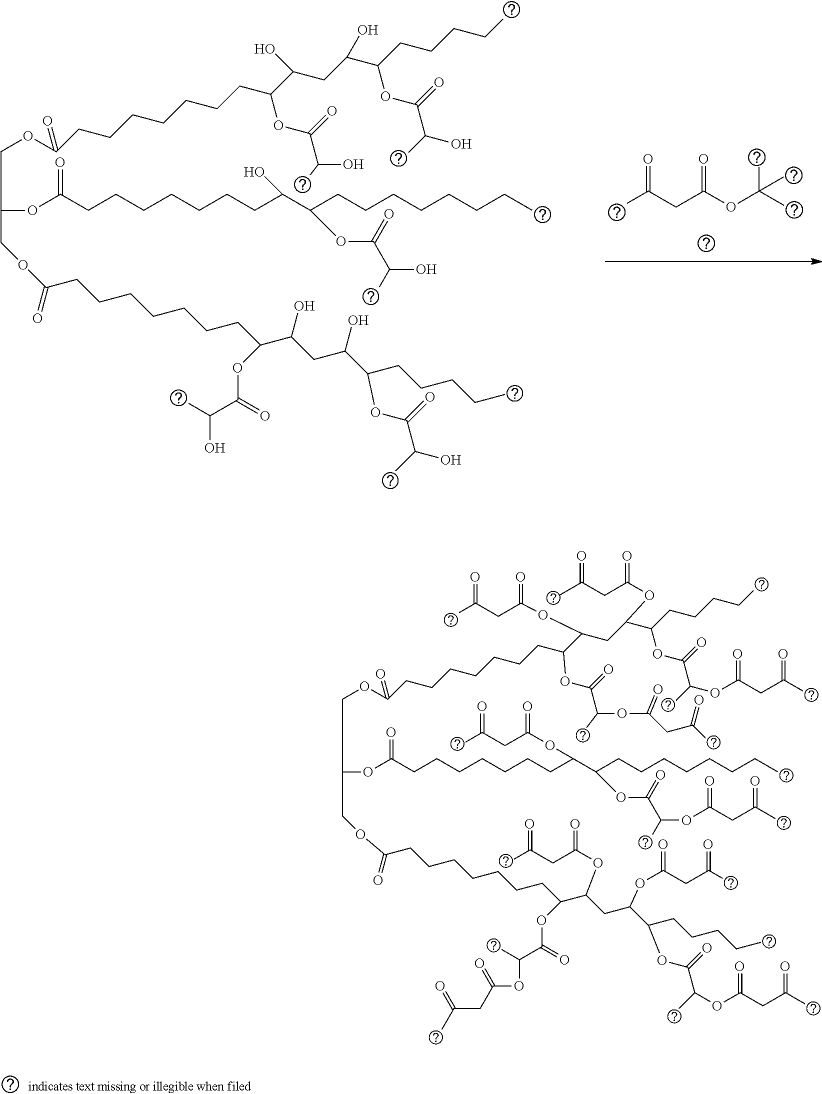 [SMILES: CCCCCCCCC(OC(=O)C(C)O)C(O)CCCCCCCC(=O)OC(COC(=O)CCCCCCCC(OC(=O)C(C)O)C(O)CC(O)C(CCCCC)OC(=O)C(C)O)COC(=O)CCCCCCCC(OC(=O)C(C)O)C(O)CC(O)C(CCCCC)OC(=O)C(C)O ; CC(=O)CC(=O)OC(C)(C)C ; CCCCCCCCC(OC(=O)C(C)OC(=O)CC(C)=O)C(CCCCCCCC(=O)OC(COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)COC(=O)CCCCCCCC(OC(=O)C(C)OC(=O)CC(C)=O)C(CC(OC(=O)CC(C)=O)C(CCCCC)OC(=O)C(C)OC(=O)CC(C)=O)OC(=O)CC(C)=O)OC(=O)CC(C)=O]

In step a), 200 g of epoxidized oil (Vikoflex 7170 sold by Arkema), 50.4 g of lactic acid and 6.52 g of caffeine are placed in a 500 ml SVL® reactor equipped with a mechanical stirrer, an argon inlet and a distillation column. The reaction mixture is heated at 120° C. for 5 hours.

The polyhydroxylated compound is obtained. A slightly yellow viscous oil is obtained.

In step b), 165 g of the polyhydroxylated compound obtained in step a) and 116.74 g of tert-butyl acetoacetate are added to a 500 ml SVL® reactor equipped with a mechanical stirrer, an argon inlet and a distillation column. The reaction medium is heated at between 130° C. and 140° C. for 5 hours. After 5 hours of heating, the medium is placed under vacuum at 50 mbar at 140° C. An orange viscous oil is obtained.

The oil obtained is diluted with ethyl acetate and washed three times with saturated sodium bicarbonate solution, and then twice with water. The organic phase is filtered over MgSO₄, and the ethyl acetate is then distilled off on a rotavapor at 100° C. under reduced pressure. A slightly orange viscous oil is obtained.

### Example 4: Synthesis of Soybean Oil Bearing an Acetoacetate Function (Compound 4)

The synthesis is represented by the following preparation scheme:

### Step a):

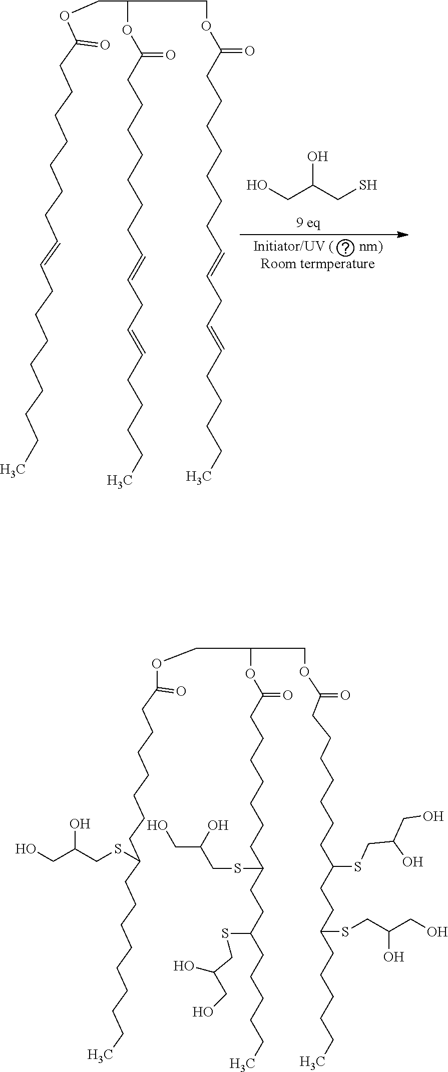 [SMILES: CCCCC/C=C/C/C=C/CCCCCCCC(=O)OCC(COC(=O)CCCCCCC/C=C/CCCCCCCC)OC(=O)CCCCCCC/C=C/C/C=C/CCCCC ; OCC(O)CS ; CCCCCCCCCC(CCCCCCCC(=O)OCC(COC(=O)CCCCCCCC(CCC(CCCCCC)SCC(O)CO)SCC(O)CO)OC(=O)CCCCCCCC(CCC(CCCCCC)SCC(O)CO)SCC(O)CO)SCC(O)CO]

### Step b):

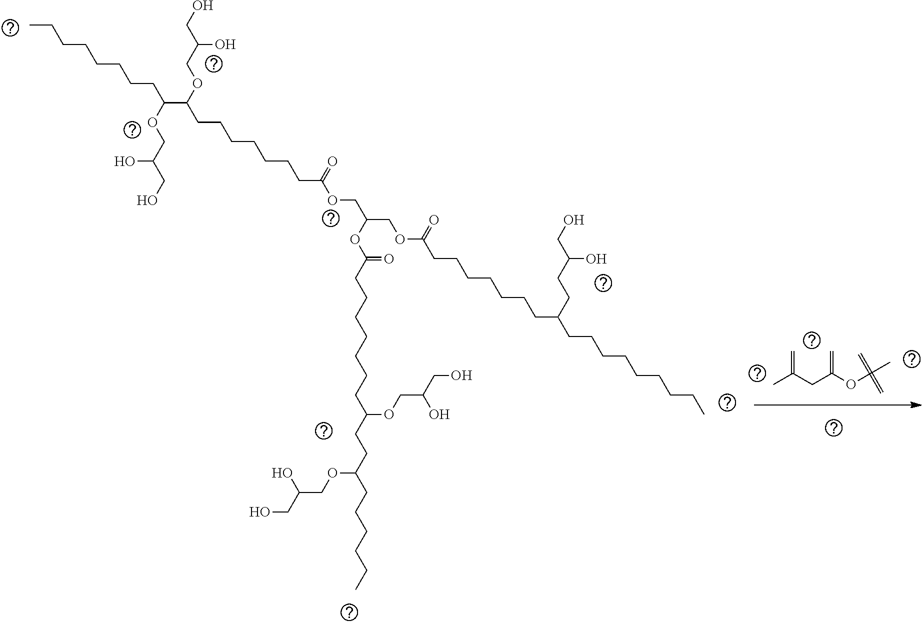

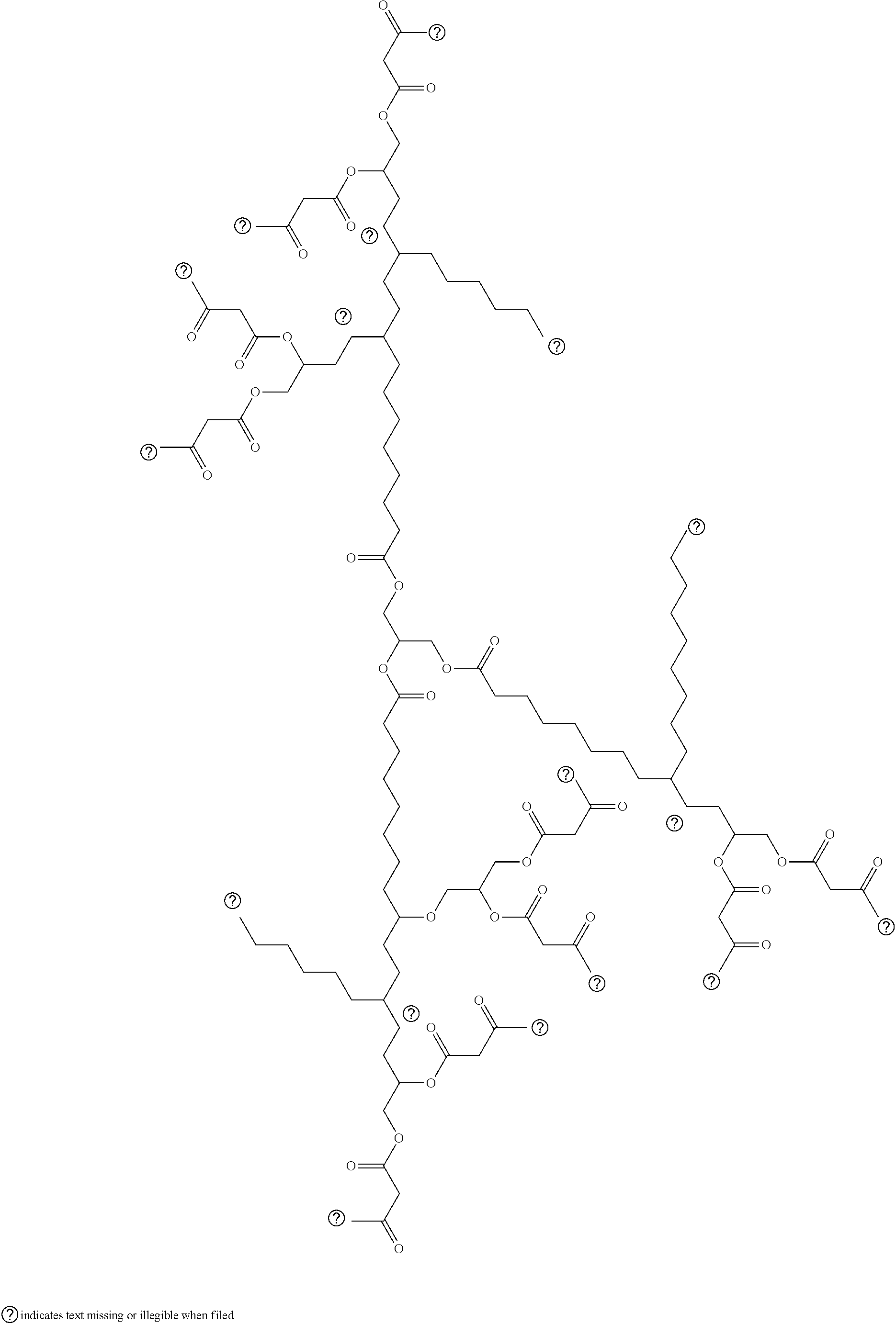 [SMILES: CCCCCCCCCC(CCCCCCCC(=O)OCC(COC(=O)CCCCCCCC(CCC(CCCCCC)CCC(COC(=O)CC(C)=O)OC(=O)CC(C)=O)CCC(COC(=O)CC(C)=O)OC(=O)CC(C)=O)OC(=O)CCCCCCCC(CCC(CCCCCC)CCC(COC(=O)CC(C)=O)OC(=O)CC(C)=O)OCC(COC(=O)CC(C)=O)OC(=O)CC(C)=O)CCC(COC(=O)CC(C)=O)OC(=O)CC(C)=O]

In step a), 10 g of soybean oil (Henry Lamotte Oils), 10 g of 3-mercapto-1,2-propanediol (sold by the company Sigma-Aldrich) and 0.8 g of 2-hydroxy-2-methylpropiophenone (sold by the company Sigma-Aldrich) are added to 25 mL of ethyl acetate in a 250 mL round-bottomed flask equipped with a magnetic stirrer, and stirred for 5 minutes under argon. The reaction medium is then irradiated under a UV lamp (100 W, 365 nm) for 4 hours, and then washed three times with NaCl-saturated water. The organic phase is separated out, dried over MgSO₄ and then concentrated under vacuum. The solvent and the traces of thiol and of photoinitiator are removed by distillation under reduced pressure.

In step b), 10 g of hydroxylated soybean oil obtained in step a) and 14 g of tert-butyl acetoacetate are placed in a 250 ml round-bottomed flask equipped with a magnetic stirrer. The reaction medium is then heated at 130° C. on an oil bath. After 4 hours, the reaction medium is cooled and placed under continuous vacuum for 1 hour so as to remove the excess tert-butyl acetoacetate. A yellow viscous oil is obtained.

### Example 5: Synthesis of an “Acidic” Oil Bearing an Acetoacetate Function (Compound 5)

The synthesis is represented by the following preparation scheme:

### Step a):

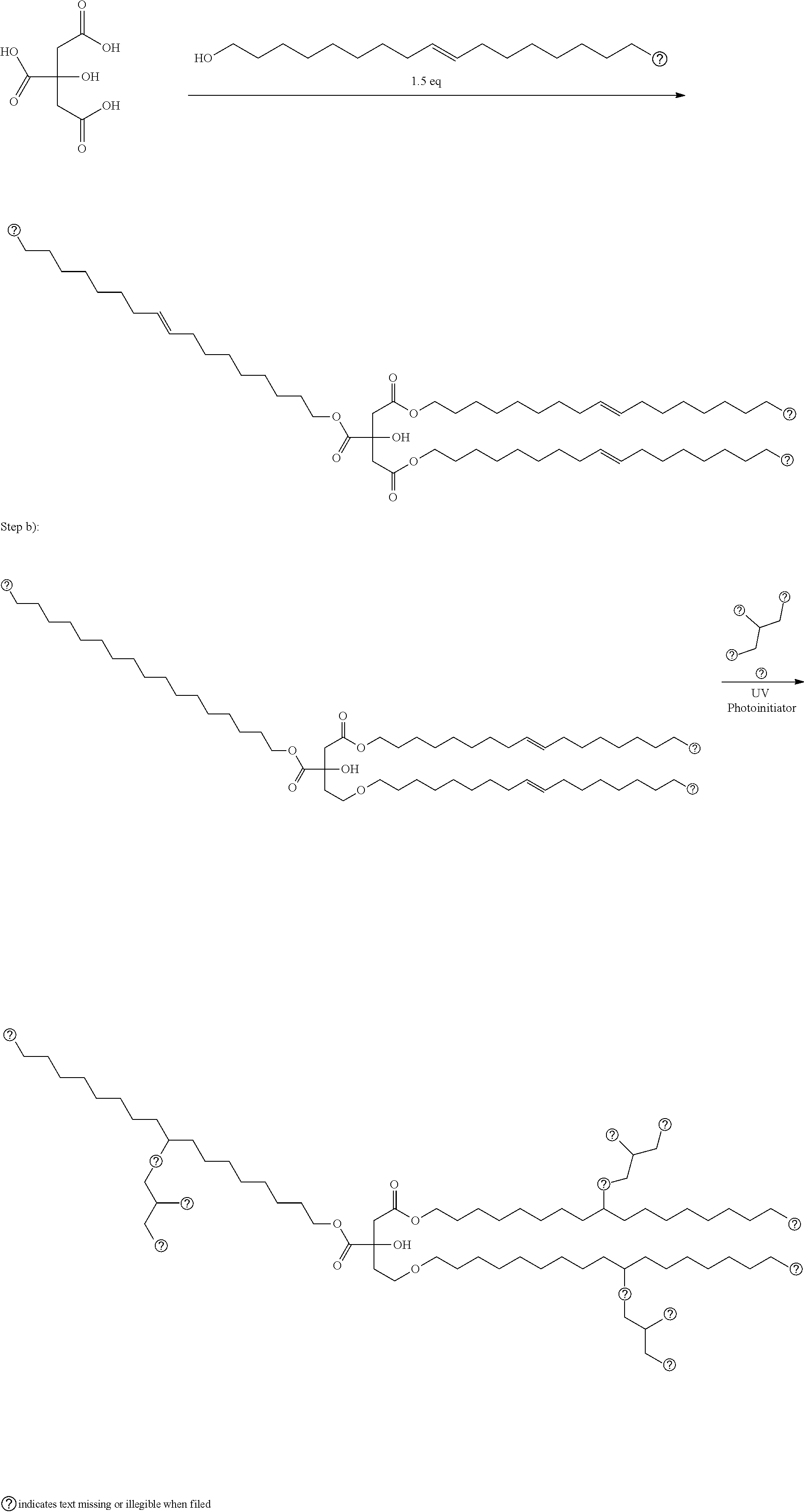

### Step c):

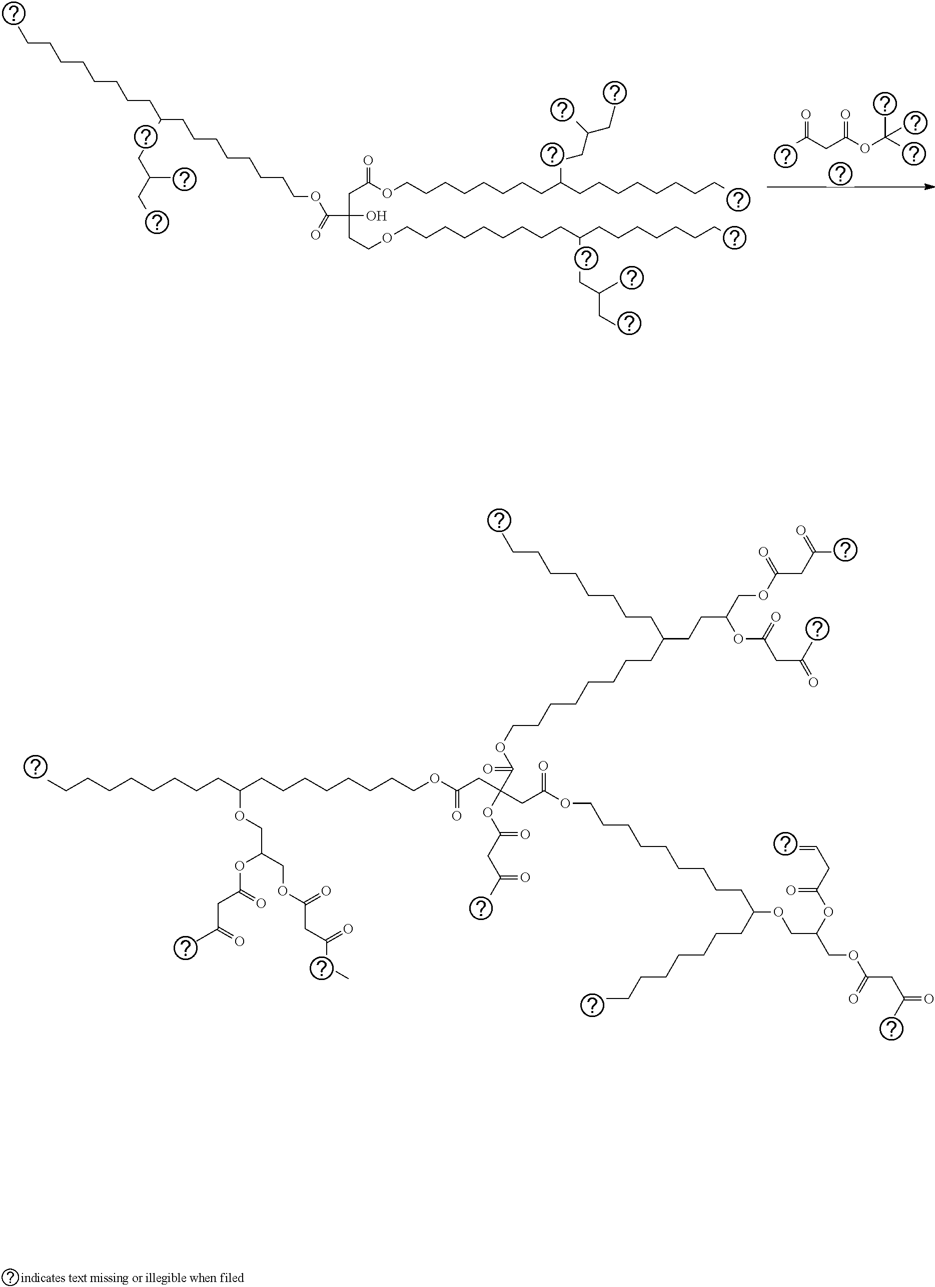

In step a), 100 g of citric acid and 419 g of oleyl alcohol are placed in a 500 mL SVL® reactor equipped with a mechanical stirrer and a distillation column. The reaction medium is heated using a heating mantle, at 190° C. for 7 hours and then at 220° C. for 5 hours. The reaction is monitored by the acid number. When the acid number is less than 5, the reaction is stopped.

In step b), 100 g of acidic oil obtained in a), 70 g of 3-mercapto-1,2-propanediol (sold by the company Sigma-Aldrich) and 5 g of 2-hydroxy-2-methylpropiophenone (sold by the company Sigma-Aldrich) are added to 15 mL of ethyl acetate in a 250 mL round-bottomed flask equipped with a magnetic stirrer, and stirred for 5 minutes under argon. The reaction medium is then irradiated under a UV lamp (100 W, 365 nm) for 4 hours and then washed three times with NaCl-saturated water. The organic phase is separated out, dried over MgSO₄ and then concentrated under vacuum. The solvent and the traces of thiol and of photoinitiator (2-hydroxy-2-methylpropiophenone) are removed by distillation under reduced pressure.

In step c), 100 g of hydroxylated acidic oil obtained in step b) and 100 g of tert-butyl acetoacetate are placed in a 250 ml round-bottomed flask equipped with a magnetic stirrer.

The reaction medium is heated at 130° C. on an oil bath. After 4 hours of reaction, the reaction medium is placed under continuous vacuum for 1 hour. A brown viscous oil is obtained.

### Example 6: Synthesis of Castor Oil Bearing an Acetoacetate Function (Compound 6)

The synthesis is represented by the following preparation scheme:

### Step a):

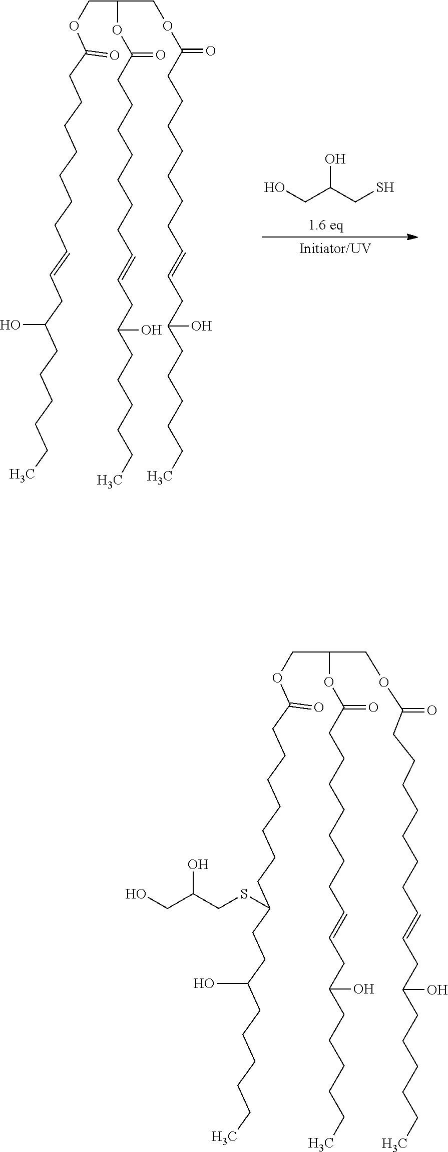 [SMILES: CCCCCCC(O)C/C=C/CCCCCCCC(=O)OCC(COC(=O)CCCCCCC/C=C/CC(O)CCCCCC)OC(=O)CCCCCCC/C=C/CC(O)CCCCCC ; OCC(O)CS ; CCCCCCC(O)C/C=C/CCCCCCCC(=O)OCC(COC(=O)CCCCCCCC(CCC(O)CCCCCC)SCC(O)CO)OC(=O)CCCCCCC/C=C/CC(O)CCCCCC]

Step b):

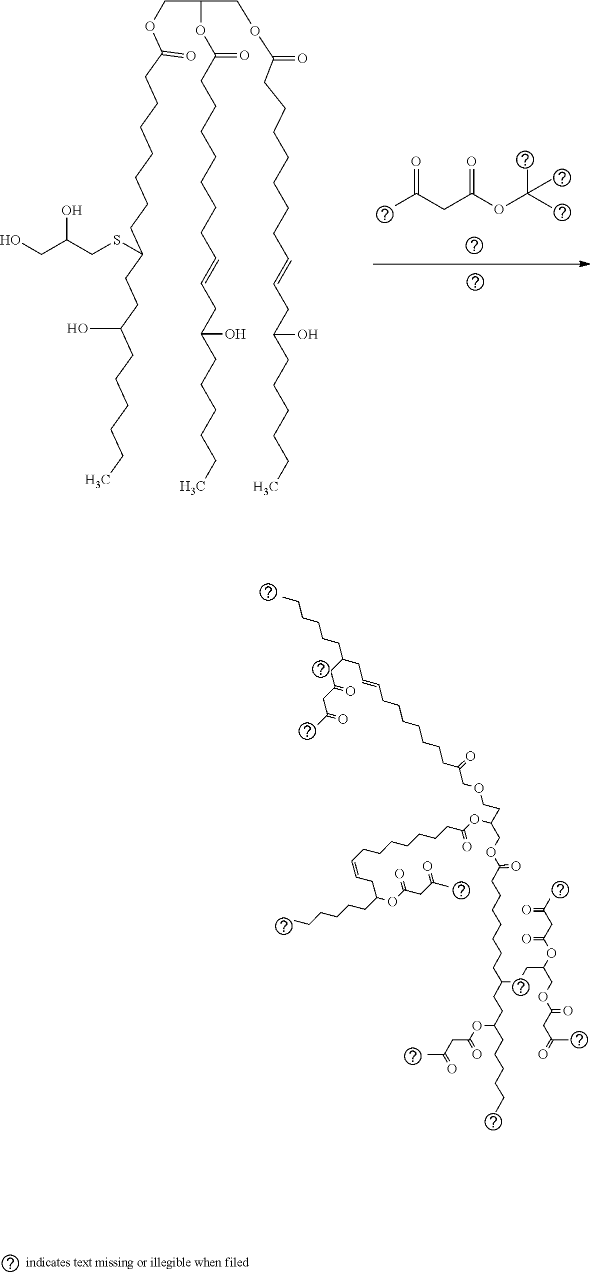 [SMILES: CCCCCCC(O)C/C=C/CCCCCCCC(=O)OCC(COC(=O)CCCCCCCC(CCC(O)CCCCCC)SCC(O)CO)OC(=O)CCCCCCC/C=C/CC(O)CCCCCC ; CC(=O)CC(=O)OC(C)(C)C ; CCCCCCC(C/C=C/CCCCCCCC(=O)COCCC(COC(=O)CCCCCCCC(CCC(CCCCCC)OC(=O)CC(C)=O)SCC(COC(=O)CC(C)=O)OC(=O)CC(C)=O)OC(=O)CCCCCCC/C=C\CC(CCCCCC)OC(=O)CC(C)=O)CC(=O)CC(C)=O]

In step a), 100 g of castor oil (sold by the company Interchimie), 17 g of 3-mercapto-1,2-propanediol (sold by the company Sigma-Aldrich) and 1 g of 2-hydroxy-2-methylpropiophenone (sold by the company Sigma-Aldrich) are added to 20 mL of ethyl acetate in a 250 mL round-bottomed flask equipped with a magnetic stirrer, and stirred for 5 minutes under argon. The reaction medium is then irradiated under a UV lamp (100 W, 365 nm) for 4 hours and then washed three times with NaCl-saturated water. The organic phase is separated out, dried over MgSO₄ and then concentrated under vacuum. The solvent and the traces of thiol and of photoinitiator (2-hydroxy-2-methylpropiophenone) are removed by distillation under reduced pressure.

In step b), 100 g of hydroxylated castor oil obtained in step a) and 99 g of tert-butyl acetoacetate are placed in a 250 ml round-bottomed flask equipped with a magnetic stirrer. The reaction medium is then heated at 130° C. on an oil bath (oil bath nominal temperature of 140° C.). After 4 hours, the reaction medium is placed under continuous vacuum for 1 hour. A yellow viscous oil is obtained.

### Example 7: Synthesis of Mannitol Dihexanoate Bearing an Acetoacetate Function (Compound 7)

The synthesis is represented by the following preparation scheme:

### Step a):

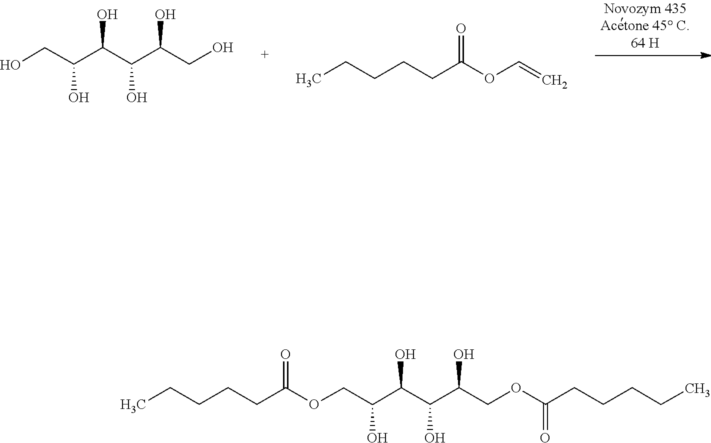

### Step b):

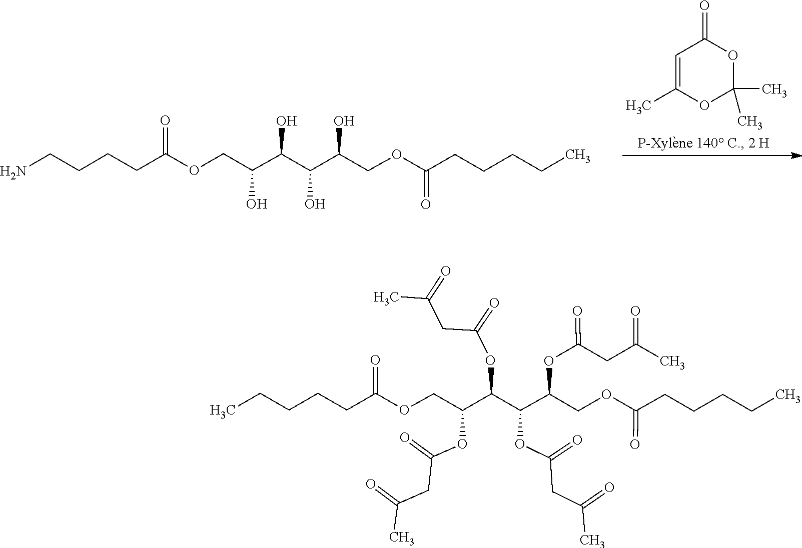

In step a), Mannitol dihexanoate is obtained from mannitol under the conditions described in the publication Angew. Chem. Int. Ed. 2010, 49, 7695-7698.

After 64 hours at 45° C., the reaction medium was filtered and then concentrated under reduced pressure. The crude compound obtained after filtration and evaporation is then triturated in dichloromethane.

In step b), 4 equivalents of 2,2,6-trimethyl-1,3-dioxine-4-one in p-xylene are heated at 140° C. for 2 hours. Purification by chromatography on silica gel leads to the compound mannitol dihexanoate acetoacetate.

### Example 8: Preparation of Compositions a to E

Composition A comprising a compound of formula (I) according to the invention, and compositions B to E comprising a compound of formula (I) according to the invention and a crosslinking agent, are prepared using the contents indicated in the table below. The contents are expressed as weight percentages relative to the total weight of the composition.

TABLE 1

| | Formula A | Formula B | Formula C | Formula D | Formula E |
|---|---|---|---|---|---|
| Compounds | (comparative) | (invention) | (invention) | (invention) | (invention) |
| Oil bearing acetoacetate | 25 | 25 | 25 | 25 | 25 |
| functions according to | | | | | |
| Example 1 | | | | | |
| Iron oxide | 3 | 3 | 3 | 3 | 3 |
| (CI: 77491, Sunpuro Red Iron | | | | | |
| Oxide C33-8001, sold by Sun) | | | | | |
| Bis-cetearyl amodimethicone | — | 5 | — | — | — |
| (sold by Momentive | | | | | |
| Performance Materials) | | | | | |
| Titanium butoxide (CAS No.: | — | — | 5 | — | — |
| 5593-70-4, sold by Sigma | | | | | |
| Aldrich) | | | | | |
| Trimethylolpropane | — | — | — | 5 | — |
| triacrylate (CAS No.: 15625- | | | | | |
| 89-5, sold by Sigma Aldrich) | | | | | |
| Terephthalaldehyde (CAS | — | — | — | — | 5 |
| No.: 623-27-8, sold by Sigma | | | | | |
| Aldrich) | | | | | |
| DBU | — | — | — | 1 | 1 |
| Isododecane/Ethanol (90/10) | qs 100 | qs 100 | qs 100 | qs 100 | qs 100 |

Compositions A to E are prepared by simply mixing the ingredients detailed in Table 1 using a Speed Mixer (mixing equipment which uses centrifugal force) for 2 minutes, at 3500 rpm and at room temperature.

### Example 9: One-Step Skin Application of Compositions a to E

Compositions A to E are applied according to the one-step skin application protocol described previously.

The deposits are then evaluated according to the protocols for resistance to olive oil, to sebum and to water, for resistance to adhesive tape, and for a fragmentation test of the formulations on Bioskin.

The evaluation results are summarized in the table below:

TABLE 2

| | Adhesive | Fragmen- | Resistance | Resistance | Resistance |
|---|---|---|---|---|---|
| Properties | tape test | tation | to olive oil | to water | to sebum |
| Formula A | 0 | 0 | 0 | 0 | 0 |
| (compar- | | | | | |
| ative) | | | | | |
| Formula B | +++ | +++ | +++ | +++ | +++ |
| (invention) | | | | | |
| Formula C | +++ | +++ | +++ | +++ | +++ |
| (invention) | | | | | |
| Formula D | Not | Not | +++ | +++ | +++ |
| (invention) | evaluated | evaluated | | | |
| Formula E | +++ | +++ | +++ | +++ | +++ |
| (invention) | | | | | |

It is seen that the application of compositions B to E according to the process of the invention allows persistence to olive oil, to water and to sebum to be obtained which is very significantly improved compared to the application of composition A, and the adhesive tape and fragmentation tests are also very impressive, thus showing that compositions B to E are cohesive and adhere to the support.

Owing to the application of compositions B to E according to the process of the invention, deposits which are resistant to everyday chemical attacks (water/sebum/olive oil) and very resistant to mechanical stresses (fragmentation) are thus obtained.

### Example 10: Preparation of Compositions F to H

Composition F comprising a compound of formula (I), and compositions G and H comprising a compound of formula (I) and a crosslinking agent, are prepared using the contents indicated in the table below. The contents are expressed as weight percentages relative to the total weight of the composition.

TABLE 3

| | Formula F | Formula G | Formula H |
|---|---|---|---|
| Compounds | (comparative) | (invention) | (invention) |
| Oil bearing acetoacetate | 10 | 10 | 10 |
| functions | | | |
| according to Example 2 | | | |
| Iron oxide | 3 | 3 | 3 |
| (CI: 77491, Sunpuro | | | |
| Red Iron Oxide | | | |
| C33-8001, sold by Sun) | | | |
| Bis-cetearyl | — | 10 | 5 |
| amodimethicone | | | |
| (sold by Momentive | | | |
| Performance | | | |
| Materials) | | | |
| Ethanol/Isododecane | qs 100 | qs 100 | qs 100 |
| (90/10) | | | |

Compositions F to H are prepared by simply mixing the ingredients detailed in Table 3 using a Speed Mixer (mixing equipment which uses centrifugal force) for 2 minutes, at 3500 rpm and at room temperature.

### Example 11: One-Step Skin Application of Compositions F to H

Compositions F to H are applied according to the one-step skin application protocol described previously.

The deposits are then evaluated according to the protocols for resistance to olive oil, to sebum and to water, and for a fragmentation test of the formulations on Bioskin.

The evaluation results are summarized in the table below:

TABLE 4

| | | Resistance | Resistance | Resistance |
|---|---|---|---|---|
| Properties | Fragmentation | to olive oil | to water | to sebum |
| Formula F | 0 | 0 | 0 | 0 |
| (comparative) | | | | |
| Formula G | +++ | +++ | +++ | +++ |
| (invention) | | | | |
| Formula H | +++ | +++ | +++ | +++ |
| (invention) | | | | |

It is seen that the application of compositions G and H according to the process of the invention allows persistence to olive oil, to water and to sebum to be obtained which is improved compared to the application of composition F, and the fragmentation tests are also very impressive.

Owing to the application of compositions G and H according to the process of the invention, deposits which are resistant to everyday chemical attacks (water/sebum/olive oil) and very resistant to mechanical stresses (fragmentation) are thus obtained.

### Example 12: Preparation of Compositions I and J

Composition I comprising a compound of formula (I), and composition J comprising a compound of formula (I) and a crosslinking agent, are prepared using the contents indicated in the table below. The contents are expressed as weight percentages relative to the total weight of the composition.

TABLE 5

| | Formula I | Formula J |
|---|---|---|
| Compounds | (comparative) | (invention) |
| Oil bearing acetoacetate functions | 10 | 10 |
| according to Example 4 | | |
| Iron oxide | 3 | 3 |
| (CI: 77491, Sunpuro Red Iron Oxide | | |
| C33-8001, sold by Sun) | | |
| Bis-cetearyl amodimethicone | — | 5 |
| (sold by Momentive Performance | | |
| Materials) | | |
| Ethanol/Isododecane (90/10) | qs 100 | qs 100 |

Compositions I and J are prepared by simply mixing the ingredients detailed in Table 5 using a Speed Mixer (mixing equipment which uses centrifugal force) for 2 minutes, at 3500 rpm and at room temperature.

### Example 13: One-Step Skin Application of Compositions I and J

Compositions I and J are applied according to the one-step skin application protocol described previously.

The deposits are then evaluated according to the protocols for resistance to olive oil, to sebum and to water, and for a fragmentation test of the formulations on Bioskin.

The evaluation results are summarized in the table below:

TABLE 6

| | | Resistance | Resistance | Resistance |
|---|---|---|---|---|
| Properties | Fragmentation | to olive oil | to water | to sebum |
| Formula I | 0 | 0 | 0 | 0 |
| (comparative) | | | | |
| Formula J | +++ | +++ | +++ | +++ |
| (invention) | | | | |

It is seen that the application of composition J according to the process of the invention allows persistence to olive oil, to water and to sebum to be obtained which is improved compared to the application of composition I, and the fragmentation tests are also very impressive.

Owing to the application of composition J according to the process of the invention, deposits which are resistant to everyday chemical attacks (water/sebum/olive oil) and very resistant to mechanical stresses (fragmentation) are thus obtained.

### Example 14: Preparation of Compositions K and L

Composition K comprising a compound of formula (I), and composition L comprising a compound of formula (I) and a crosslinking agent, are prepared using the contents indicated in the table below. The contents are expressed as weight percentages relative to the total weight of the composition.

TABLE 7

| | Formula K | Formula L |
|---|---|---|
| Compounds | (comparative) | (invention) |
| Oil bearing acetoacetate functions | 95 | 66.5 |
| according to Example 5 | | |
| Yellow iron oxide | 5 | 5 |
| (Sunpuro Yellow Iron Oxide C33- | | |
| 9001) | | |
| 4,7,10-Trioxa-1,13-tridecanediamine | — | 28.5 |
| (CAS number: 4246-51-9, sold by | | |
| Sigma Aldrich) | | |

Compositions K and L are prepared by simply mixing the ingredients detailed in Table 7 using a Speed Mixer (mixing equipment which uses centrifugal force) for 2 minutes, at 3500 rpm and at room temperature.

### Example 15: One-Step Skin Application of Compositions K and L

Compositions K and L are applied according to the one-step skin application protocol described previously.

The deposits are then evaluated according to the protocols for resistance to olive oil, to sebum and to water, for resistance to adhesive tape, and for a fragmentation test of the formulations on Bioskin.

The evaluation results are summarized in the table below:

TABLE 8

| | Adhesive | Fragmen- | Resistance | Resistance | Resistance |
|---|---|---|---|---|---|
| Properties | tape test | tation | to olive oil | to water | to sebum |
| Formula K | 0 | 0 | 0 | 0 | 0 |
| (compar- | | | | | |
| ative) | | | | | |
| Formula L | +++ | +++ | +++ | +++ | +++ |
| (invention) | | | | | |

It is seen that the application of composition L according to the process of the invention allows persistence to olive oil, to water and to sebum to be obtained which is improved compared to the application of composition K, and the adhesive tape and fragmentation tests are also very impressive, thus showing that composition L is cohesive and adheres to the support.

Owing to the application of composition L according to the process of the invention, deposits which are resistant to everyday chemical attacks (water/sebum/olive oil) and very resistant to mechanical stresses (fragmentation) are thus obtained.

### Example 16: Preparation of Compositions M and N

Composition M comprising a compound of formula (I), and composition N comprising a compound of formula (I) and a crosslinking agent, are prepared using the contents indicated in the table below. The contents are expressed as weight percentages relative to the total weight of the composition.

TABLE 9

| | Formula M | Formula N |
|---|---|---|
| Compounds | (comparative) | (invention) |
| Oil bearing acetoacetate functions | 95 | 87.9 |
| according to Example 6 | | |
| DC Red 7 Lake | 5 | 5 |
| (Sensient) | | |
| 4,7,10-Trioxa-1,13-tridecanediamine | — | 7.1 |
| (CAS number: 4246-51-9, sold by | | |
| Sigma Aldrich) | | |

Compositions M and N are prepared by simply mixing the ingredients detailed in Table 9 using a Speed Mixer (mixing equipment which uses centrifugal force) for 2 minutes, at 3500 rpm and at room temperature.

### Example 17: One-Step Skin Application of Compositions M and N

Compositions M and N are applied according to the one-step skin application protocol described previously.

The deposits are then evaluated according to the protocols for resistance to olive oil, to sebum and to water, for resistance to adhesive tape, and for a fragmentation test of the formulations on Bioskin.

The evaluation results are summarized in the table below:

TABLE 10

| | Adhesive | Fragmen- | Resistance | Resistance | Resistance |
|---|---|---|---|---|---|
| Properties | tape test | tation | to olive oil | to water | to sebum |
| Formula M | 0 | 0 | 0 | 0 | 0 |
| (compar- | | | | | |
| ative) | | | | | |
| Formula N | +++ | +++ | +++ | +++ | ++ |
| (invention) | | | | | |

It is seen that the application of composition N according to the process of the invention allows persistence to olive oil, to water and to sebum to be obtained which is improved compared to the application of composition M, and the adhesive tape and fragmentation tests are also very impressive, thus showing that composition N is cohesive and adheres to the support.

Owing to the application of composition N according to the process of the invention, deposits which are resistant to everyday chemical attacks (water/sebum/olive oil) and very resistant to mechanical stresses (fragmentation) are thus obtained.

### Example 18: Preparation of Compositions O to R

Compositions O to R are prepared using the contents indicated in the table below. The contents are expressed as weight percentages relative to the total weight of the composition under consideration.

TABLE 11

| | | | Formulas | Formula | Formulas |
|---|---|---|---|---|---|
| | | Formula O | P (Base | Q | R (Base |
| | | (Base | coat + | (Base | coat + |
| | | coat) | Top coat) | coat) | Top coat) |
| | | (compar- | (inven- | (inven- | (inven- |
| | Compounds | ative) | tion) | tion) | tion) |
| Base | Oil bearing | 20 | 25 | 25 | 25 |
| coat | acetoacetate | | | | |
| | functions | | | | |
| | according to | | | | |
| | Example 2 | | | | |
| | Iron oxide | 3 | 5 | 5 | 5 |
| | (CI: 77491, | | | | |
| | Sunpuro Red | | | | |
| | Iron Oxide | | | | |
| | C33-8001, | | | | |
| | sold by Sun) | | | | |
| | Spermidine | — | 5 | 5 | 5 |
| | (CAS number: | | | | |
| | 124-20-9, sold | | | | |
| | by Sigma-Aldrich) | | | | |
| | Ethanol/Isododecane | qs 100 | qs 100 | qs 100 | qs 100 |
| | (50/50) | | | | |
| Top | (3-Aminopropyl) | — | 5 | — | — |
| coat | triethoxysilane | | | | |
| | (APTES) (CAS | | | | |
| | number: 919-30- | | | | |
| | 2, sold by | | | | |
| | Sigma Aldrich) | | | | |
| | [4-6% | — | — | — | 5 |
| | (Mercaptopropyl) | | | | |
| | Methylsiloxane]- | | | | |
| | Dimethylsiloxane | | | | |
| | copolymer | | | | |
| | (CAS Number: | | | | |
| | 102783-03-9), | | | | |
| | sold by Gelest, | | | | |
| | molecular weight | | | | |
| | 6,000-8,000) | | | | |
| | Isododecane | — | qs 100 | — | qs 100 |

The “base coat” and “top coat” compositions are prepared by simply mixing the ingredients detailed in Table 11 using a Speed Mixer (mixing equipment which uses centrifugal force) for 2 minutes, at 3500 rpm and at room temperature.

### Example 19: One-Step or Two-Step Skin Application of Compositions O to R

Compositions O to R are applied according to the one-step or two-step skin application protocol described previously.

The deposits are then evaluated according to the protocols for resistance to olive oil, to sebum and to water, for resistance to adhesive tape, and for a fragmentation test of the formulations on Bioskin.

The evaluation results are summarized in the table below:

TABLE 12

| | Adhesive | Fragmen- | Resistance | Resistance | Resistance |
|---|---|---|---|---|---|
| Properties | tape test | tation | to olive oil | to water | to sebum |
| Formula O | 0 | 0 | 0 | 0 | 0 |
| (compar- | | | | | |
| ative) | | | | | |
| Formulas P | +++ | ++ | +++ | +++ | ++ |
| (invention) | | | | | |
| Formulas Q | +++ | + | +++ | +++ | +++ |
| (invention) | | | | | |
| Formulas R | +++ | + | +++ | +++ | +++ |
| (invention) | | | | | |

It is seen that the application of the formulas P to R according to the process of the invention allows persistence to olive oil, to water and to sebum to be obtained which is improved compared to the application of composition O, and the adhesive tape and fragmentation tests are also very impressive, thus showing that compositions P to R are cohesive and adhere to the support.

Owing to the application of formulas P to R according to the process of the invention, deposits which are resistant to everyday chemical attacks (water/sebum/olive oil) and also a tensor effect on the Bioskin are thus obtained.

### Example 20: Preparation of Compositions S and T

Composition S comprising a compound of formula (I), and composition T comprising a compound of formula (I) and a crosslinking agent, are prepared using the contents indicated in the table below. The contents are expressed as weight percentages relative to the total weight of the composition.

TABLE 13

| | Formula S | Formula T |
|---|---|---|
| Compounds | (comparative) | (invention) |
| Oil bearing acetoacetate functions | 20 | 20 |
| according to Example 7 | | |
| D&C Red 28 Lake - Phloxine B Di- | 5 | 5 |
| Sodium Salt Aluminium Lake On | | |
| Alumina, Aluminium Benzoate | | |
| (CI 45410:2 + 77002) | | |
| Titanium butoxide (CAS number: | — | 5 |
| 5593-70-4, sold by Sigma Aldrich) | | |
| Ethanol/Isododecane (70/30) | 75 | 70 |

Compositions S and T are prepared by simply mixing the ingredients detailed in Table 13 using a Speed Mixer (mixing equipment which uses centrifugal force) for 2 minutes, at 3500 rpm and at room temperature.

### Example 21: One-Step Skin Application of Compositions S and T

Compositions S and T are applied according to the one-step skin application protocol described previously.

The deposits are then evaluated according to the protocols for resistance to olive oil, to sebum and to water, for resistance to adhesive tape, and for a fragmentation test of the formulations on Bioskin.

The evaluation results are summarized in the table below:

TABLE 14

| | Adhesive | Fragmen- | Resistance | Resistance | Resistance |
|---|---|---|---|---|---|
| Properties | tape test | tation | to olive oil | to water | to sebum |
| Formula S | 0 | 0 | 0 | 0 | 0 |
| (compar- | | | | | |
| ative) | | | | | |
| Formula T | ++ | +++ | ++ | ++ | ++ |
| (invention) | | | | | |

It is seen that the application of composition T according to the process of the invention allows persistence to olive oil, to water and to sebum to be obtained which is improved compared to the application of composition S, and the adhesive tape and fragmentation tests are also very impressive, thus showing that composition T is cohesive and adheres to the support.

Owing to the application of composition T according to the process of the invention, deposits which are resistant to everyday chemical attacks (water/sebum/olive oil) and very resistant to mechanical stresses (fragmentation) are thus obtained.

### Example 22: Preparation of Compositions U and V

Composition U comprising a compound of formula (I), and composition V comprising a compound of formula (I) and a crosslinking agent, are prepared using the contents indicated in the table below. The contents are expressed as weight percentages relative to the total weight of the composition.

TABLE 15

| | Formula U | Formula V |
|---|---|---|
| Compounds | (comparative) | (invention) |
| Oil bearing acetoacetate functions | 20 | 20 |
| according to Example 7 | | |
| D&C Red 28 Lake - Phloxine B Di- | 5 | 5 |
| Sodium Salt Aluminium Lake On | | |
| Alumina, Aluminium Benzoate | | |
| (CI 45410:2 + 77002) | | |
| 4,7,10-Trioxa-1,13-tridecanediamine | — | 5 |
| (CAS number: 4246-51-9, sold by | | |
| Sigma Aldrich) | | |
| Ethanol/Isododecane (70/30) | 75 | 70 |

Compositions U and V are prepared by simply mixing the ingredients detailed in Table 15 using a Speed Mixer (mixing equipment which uses centrifugal force) for 2 minutes, at 3500 rpm and at room temperature.

### Example 23: One-Step Skin Application of Compositions U and V

Compositions U and V are applied according to the one-step skin application protocol described previously.

The deposits are then evaluated according to the protocols for resistance to olive oil, to sebum and to water, and for a fragmentation test of the formulations on Bioskin.

The evaluation results are summarized in the table below:

TABLE 16

| | | Resistance | Resistance | Resistance |
|---|---|---|---|---|
| Properties | Fragmentation | to olive oil | to water | to sebum |
| Formula U | 0 | 0 | 0 | 0 |
| (comparative) | | | | |
| Formula V | ++ | +++ | ++ | ++ |
| (invention) | | | | |

It is seen that the application of composition V according to the process of the invention allows persistence to olive oil, to water and to sebum to be obtained which is improved compared to the application of composition U, and the fragmentation tests are also very impressive.

Owing to the application of composition V according to the process of the invention, deposits which are resistant to everyday chemical attacks (water/sebum/olive oil) and very resistant to mechanical stresses (fragmentation) are thus obtained.

1-24. (canceled)

25. A method for treating keratin materials, comprising applying to the keratin materials a composition comprising:
i) one or more compounds of formula (I), optical or geometrical isomers thereof, solvates thereof, or mixtures thereof: 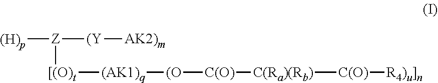
wherein in formula (I):
Z represents a linear or branched, saturated or unsaturated, conjugated or non-conjugated, acyclic or cyclic, aromatic or non-aromatic multivalent C₂ to C₄₀, hydrocarbon-based radical, Z possibly being interrupted with one or more groups or heteroatoms chosen from —O—, —N(Rₐ)—, —S—, —C(O)—, (hetero)cycles, or combinations thereof;
AK1 represents a divalent saturated or unsaturated, linear or branched C₁-C₁₂, hydrocarbon-based chain;
AK2 represents a linear or branched, saturated or unsaturated, C₁-C₄₀ monovalent hydrocarbon-based chain, optionally substituted with one or more radicals independently chosen from —OR; —X-AK3-(O—C(O)—C(Rₐ)(R_{b})—C(O)—R₄)ᵥ; or said chain being optionally interrupted with one or more heteroatoms or groups chosen from —O—, —S—, —C(O)—, (hetero)cycle(s), or combinations thereof;
AK3 represents a divalent linear or branched, saturated or unsaturated C₁-C₁₂, hydrocarbon-based chain;
R represents a hydrogen atom, a linear or branched, saturated or unsaturated C₁-C₆, alkyl radical, or a radical —C(O)—C(Rₐ)(R_{b})—C(O)—R₄;
X represents —O—, —S—, —O—C(O)— or —C(O)—O—;
Y represents —O—C(O)— or —C(O)—O—;
R₄ represents a linear or branched, saturated or unsaturated C₁-C₆ monovalent hydrocarbon-based radical;
Rₐ and R_{b} are independently chosen from a hydrogen atom or a (C₁-C₄)alkyl group;
p denotes an integer equal to 0 or 1;
m denotes an integer equal to 0, 1, 2, 3 or 4; wherein when m is greater than 1, then the radicals —Y-AK₂ are identical or different;
n denotes an integer equal to 0, 1, 2, 3 or 4; wherein when n is greater than 1, then the radicals —(O)ₜ-(AK₁)_{q}-(O—C(O)—C(Rₐ)(R_{b})—C(O)—R₄)ᵤ are identical or different;
t denotes an integer equal to 0 or 1;
q denotes an integer equal to 0 or 1;
u denotes an integer equal to 1, 2 or 3;
v denotes an integer equal to 1, 2 or 3;
with the proviso that if q is equal to 0 then t is equal to 0, that (m+n) is greater than or equal to 1, and that the compounds of formula (I) comprise at least two groups —C(O)—C(Rₐ)(R_{b})—C(O)—R₄; and
ii) one or more crosslinking agents.

26. The method of claim 25, wherein the (a) one or more compounds of formula (I), optical or geometrical isomers thereof, solvates thereof, or mixtures thereof is chosen from compound(s) s of formula (I), wherein in formula (I):
Z represents the trivalent radical —CH₂—CH—CH₂—;
p is equal to 0;
n is equal to 0;
m is equal to 3;
AK2 represents a linear or branched, saturated or unsaturated, C₁₂-C₂₄ monovalent hydrocarbon-based chain, optionally substituted with one or more identical or different radicals chosen from —OR or —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, epoxides, or combinations thereof;
wherein said compound comprises at least two radicals —C(O)—C(Rₐ)(R_{b})—C(O)—R₄.

27. The method of claim 25, wherein the (a) one or more compounds of formula (I), optical or geometrical isomers thereof, solvates thereof, or mixtures thereof is chosen from compounds of formula (I), wherein in formula (I):
p is equal to 0;
Z represents a linear or branched, saturated or unsaturated, acyclic, multivalent C₄ to C₂₄, hydrocarbon-based radical, Z being optionally substituted with one or more radicals —[(O)ₜ-(AK1)_{q}-(O—C(O)—C(Rₐ)(R_{b})—C(O)—R₄)ᵤ]ₙ;
n denotes an integer equal to 0, 1, 2, 3 or 4; with the proviso that when n is greater than 1, then the radicals —(O)ₜ-(AK₁)_{q}-(O—C(O)—C(Rₐ)(R_{b})—C(O)—R₄)ᵤ are identical or different;
m is equal to 1;
AK2 represents a linear or branched, saturated or unsaturated, C₃-C₈, monovalent hydrocarbon-based chain, substituted with one or more identical or different radicals chosen from —OR, —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), or combinations thereof;
wherein said compound comprises at least two radicals —C(O)—C(Rₐ)(R_{b})—C(O)—R₄.

28. The method of claim 25, wherein in the (i) one or more compounds of formula (I):
p is equal to 0;
n+m is equal to 4, m being non-zero;
Z represents an unsubstituted and uninterrupted, linear or branched, saturated or unsaturated, and acyclic, C₂ to C₂₄, tetravalent hydrocarbon-based radical;
AK2 independently represent a linear or branched, saturated or unsaturated, C12-C24, monovalent hydrocarbon-based chain, optionally substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), or combinations thereof;
AK3 represents a linear or branched, saturated or unsaturated C1-C12divalent hydrocarbon-based chain;
wherein said compound comprises at least two radicals —C(O)—C(Rₐ)(R_{b})—C(O)—R₄.

29. The method of claim 25, wherein the (a) one or more compounds of formula (I), optical or geometrical isomers thereof, solvates thereof, or mixtures thereof is chosen from compounds of formula (I), wherein in formula (I):
p is equal to 1;
Z represents a linear or branched, saturated or unsaturated, preferably saturated and acyclic, multivalent C₁₄ to C₂₄ hydrocarbon-based radical, Z being optionally substituted with one or more radicals —[(O)ₜ-(AK1)_{q}-(O—C(O)—C(Rₐ)(R_{b})—C(O)—R₄)ᵤ]ₙ;
AK2 represents a linear or branched, saturated or unsaturated monovalent hydrocarbon-based chain, substituted with one or more identical or different radicals chosen from —OR, —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), and combinations thereof;
wherein said compound contains at least two radicals —C(O)—C(Rₐ)(R_{b})—C(O)—R₄.

30. The method of claim 25, wherein the (a) one or more compounds of formula (I), optical or geometrical isomers thereof, solvates thereof, or mixtures thereof is chosen from compounds of formula (I), wherein:
p is equal to 0;
Z represents a linear or branched, saturated or unsaturated, acyclic, C₂ to C₂₄, tetravalent hydrocarbon-based radical, Z being substituted with one or more radicals —[(O)ₜ-(AK1)_{q}-(O—C(O)—C(Rₐ)(R_{b})—C(O)—R₄)ᵤ]ₙ;
n+m is equal to 4;
AK2 independently represents a linear or branched, saturated or unsaturated, C₁₄ to C₃₀, hydrocarbon-based chain, optionally substituted with one or more identical or different radicals chosen from —OR, —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), or combinations thereof;
wherein said compound comprises at least two radicals —C(O)—C(Rₐ)(R_{b})—C(O)—R₄.

31. The method of claim 25, wherein the (a) one or more compounds of formula (I), optical or geometrical isomers thereof, solvates thereof, or mixtures thereof is chosen from compounds of formula (I), wherein in formula (I):
p is equal to 0;
m is non-zero;
Z represents a linear or branched, saturated or unsaturated, conjugated or non-conjugated, acyclic or cyclic, aromatic or non-aromatic, divalent C₂ to C₂₄, hydrocarbon-based radical, said radical Z also being interrupted with one or more heteroatoms or groups chosen from —O—, —N(Rₐ)—, —S—, —C(O)—, 5- or 6-membered heterocycloalkyl(s), or combinations thereof;
AK2 represents a linear or branched, saturated or unsaturated, C₁₄-C₃₀, monovalent hydrocarbon-based chain, optionally substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycles, or combinations thereof;
wherein said compound comprises at least two radicals —C(O)—C(Rₐ)(R_{b})—C(O)—R₄.

32. The method of claim 25, in wherein the (a) one or more compounds of formula (I), optical or geometrical isomers thereof, solvates thereof, or mixtures thereof is chosen from compounds of formula (I), wherein in formula (I):
p is equal to 0;
Z represents a linear or branched, saturated or unsaturated, and acyclic, C₂ to C₂₄, tetravalent hydrocarbon-based radical, Z being substituted with one or more radicals —[(O)t-(AK1)q-(O—C(O)—C(Ra)(Rb)—C(O)—R4)u]n;
n+m is equal to 6;
AK2 represents a linear or branched, saturated or unsaturated, C₃-C₈, monovalent hydrocarbon-based chain;
wherein said compound comprises at least two radicals —C(O)—C(Rₐ)(R_{b})—C(O)—R₄.

33. The method of claim 25, wherein the (a) one or more compounds of formula (I), optical or geometrical isomers thereof, solvates thereof, or mixtures thereof is chosen from compounds of formula (Ia) optical or geometrical isomers thereof, solvates thereof, or mixtures thereof: 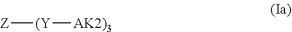
wherein in formula (Ia):
Z represents —CH₂—CH—CH₂—;
AK2 independently denotes a linear or branched, saturated or unsaturated, C12-C24 monovalent hydrocarbon-based chain, optionally substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, epoxides, or combinations thereof;
wherein said compound comprises at least two radicals —C(O)—C(Rₐ)(R_{b})—C(O)—R₄.

34. The method of claim 25, wherein the (a) one or more compounds of formula (I), optical or geometrical isomers thereof, solvates thereof, or mixtures thereof is chosen from compounds of formula (Ib), optical or geometrical isomers thereof, solvates thereof, or mixtures thereof: 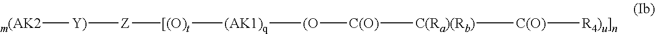
wherein in formula (Ib):
n+m is equal to 4, m being non-zero;
Z represents an unsubstituted and uninterrupted, linear or branched, saturated or unsaturated, and acyclic, C₂ to C₂₄, tetravalent hydrocarbon-based radical;
AK2 independently represent a linear or branched, saturated or unsaturated, C₁₂-C₂₄, monovalent hydrocarbon-based chain, optionally substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), or combinations thereof;
AK3 represents a linear or branched, saturated or unsaturated C₁-C₁₂ divalent hydrocarbon-based chain;
wherein said compound comprises at least two radicals —C(O)—C(Rₐ)(R_{b})—C(O)—R₄.

35. The method of claim 25, wherein the (a) one or more compounds of formula (I), optical or geometrical isomers thereof, solvates thereof, or mixtures thereof is chosen from compounds of formula (Ic), optical or geometrical isomers thereof, solvates thereof, or mixtures thereof: 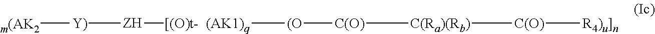
wherein in formula (Ic):
Z represents a linear or branched, saturated or unsaturated, and acyclic, multivalent C₁₄ to C₂₄ hydrocarbon-based radical, Z being optionally substituted with one or more radicals —[(O)ₜ-(AK1)_{q}-(O—C(O)—C(Rₐ)(R_{b})—C(O)—R₄)ᵤ]ₙ;
AK2 represents a linear or branched, saturated or unsaturated, C₁₄-C₃₀, monovalent hydrocarbon-based chain, substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), or combinations thereof;
wherein said compound comprises at least two radicals —C(O)—C(Rₐ)(R_{b})—C(O)—R₄.

36. The method of claim 25, wherein the (a) one or more compounds of formula (I), optical or geometrical isomers thereof, solvates thereof, or mixtures thereof is chosen from compounds of formula (Id), optical or geometrical isomers thereof, solvates thereof, or mixtures thereof: 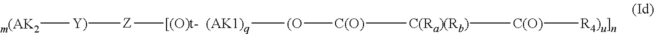
wherein in formula (Id):
n+m is equal to 4 3;
Z represents a linear or branched, saturated or unsaturated, or acyclic, C₂ to C₂₄, tetravalent hydrocarbon-based radical, Z also being substituted with one or more radicals —[(O)ₜ-(AK1)_{q}-(O—C(O)—C(Rₐ)(R_{b})—C(O)—R₄)ᵤ]ₙ;
AK2 independently represents a linear or branched, saturated or unsaturated, C₁₄-C₃₀ monovalent hydrocarbon-based chain, optionally substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycle(s), or combinations thereof;
wherein said compound comprises at least two radicals —C(O)—C(Rₐ)(R_{b})—C(O)—R₄.

37. The method of claim 25, wherein the (a) one or more compounds of formula (I), optical or geometrical isomers thereof, solvates thereof, or mixtures thereof is chosen from compounds of formula (Ie), optical or geometrical isomers thereof, solvates thereof, or mixtures thereof: 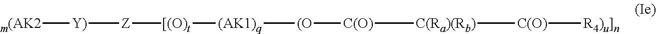
wherein in formula (Ie):
n+m is equal to 2, m being non-zero;
Z represents a linear or branched, saturated or unsaturated, conjugated or non-conjugated, acyclic or cyclic, aromatic or non-conjugated, C₂ to C₂₄ divalent hydrocarbon-based radical, said radical Z being interrupted with one or more heteroatoms or groups chosen from —O—, —N(Rₐ)—, —S—, —C(O)—, (hetero)cycle(s), or combinations thereof; said heterocycle being optionally substituted with one or more radicals chosen from —OR;
AK2 represents a linear or branched, saturated or unsaturated, C₁₄-C₃₀, monovalent hydrocarbon-based chain, optionally substituted with one or more identical or different radicals chosen from —OR and —X-AK3-(O—C(O)—C(Rₐ)(R_{b})—C(O)—R₄)ᵥ; and/or said chain being optionally interrupted with one or more heteroatoms or groups chosen from —O—, —S—, —C(O)—, (hetero)cycles, or combinations thereof;
wherein said compound comprises at least two radicals —C(O)—C(Rₐ)(R_{b})—C(O)—R₄.

38. The method of claim 25, wherein the (a) one or more compounds of formula (I), optical or geometrical isomers thereof, solvates thereof, or mixtures thereof is chosen from compounds of (1) through (15): 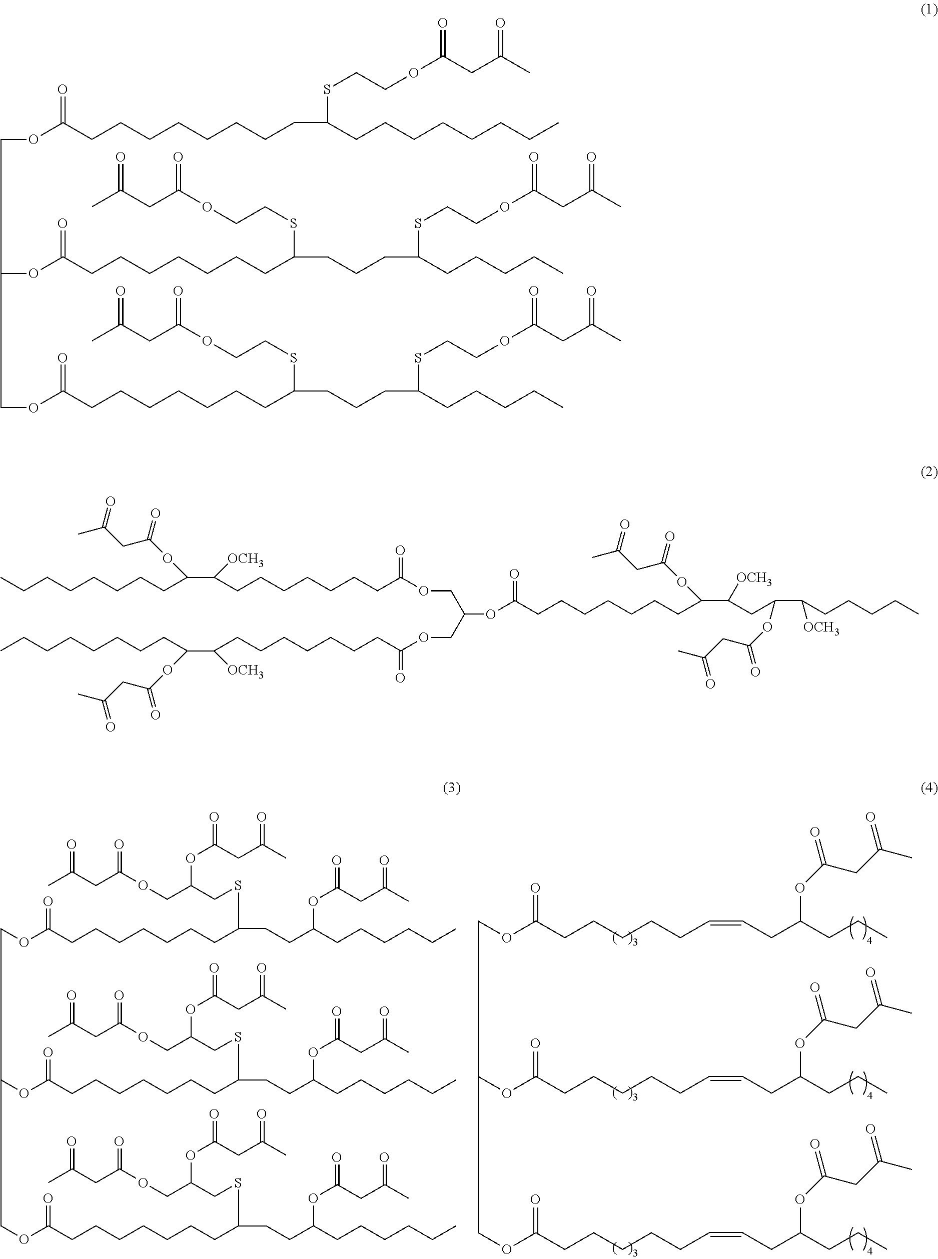 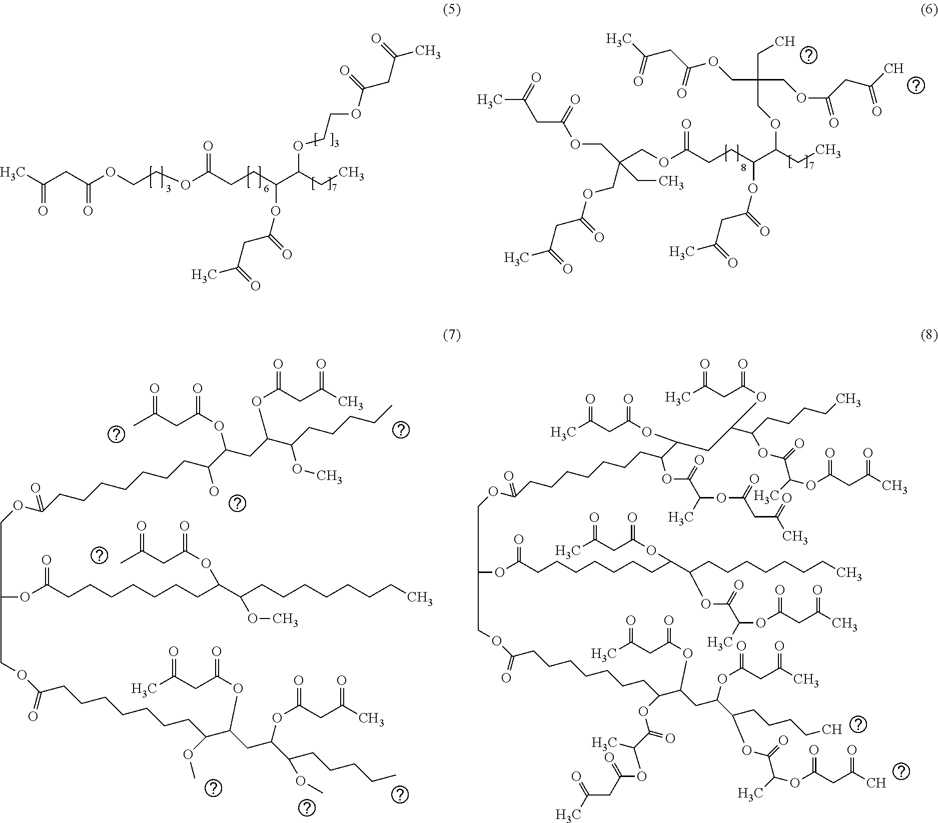 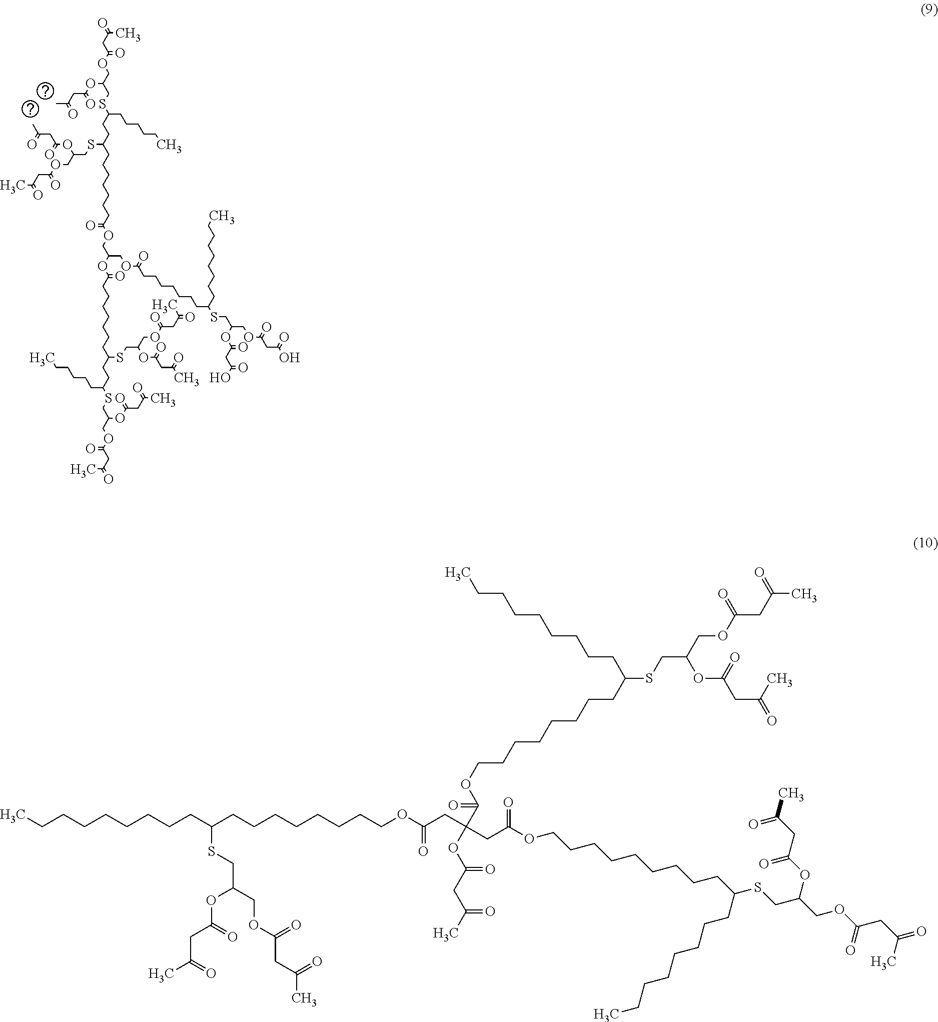 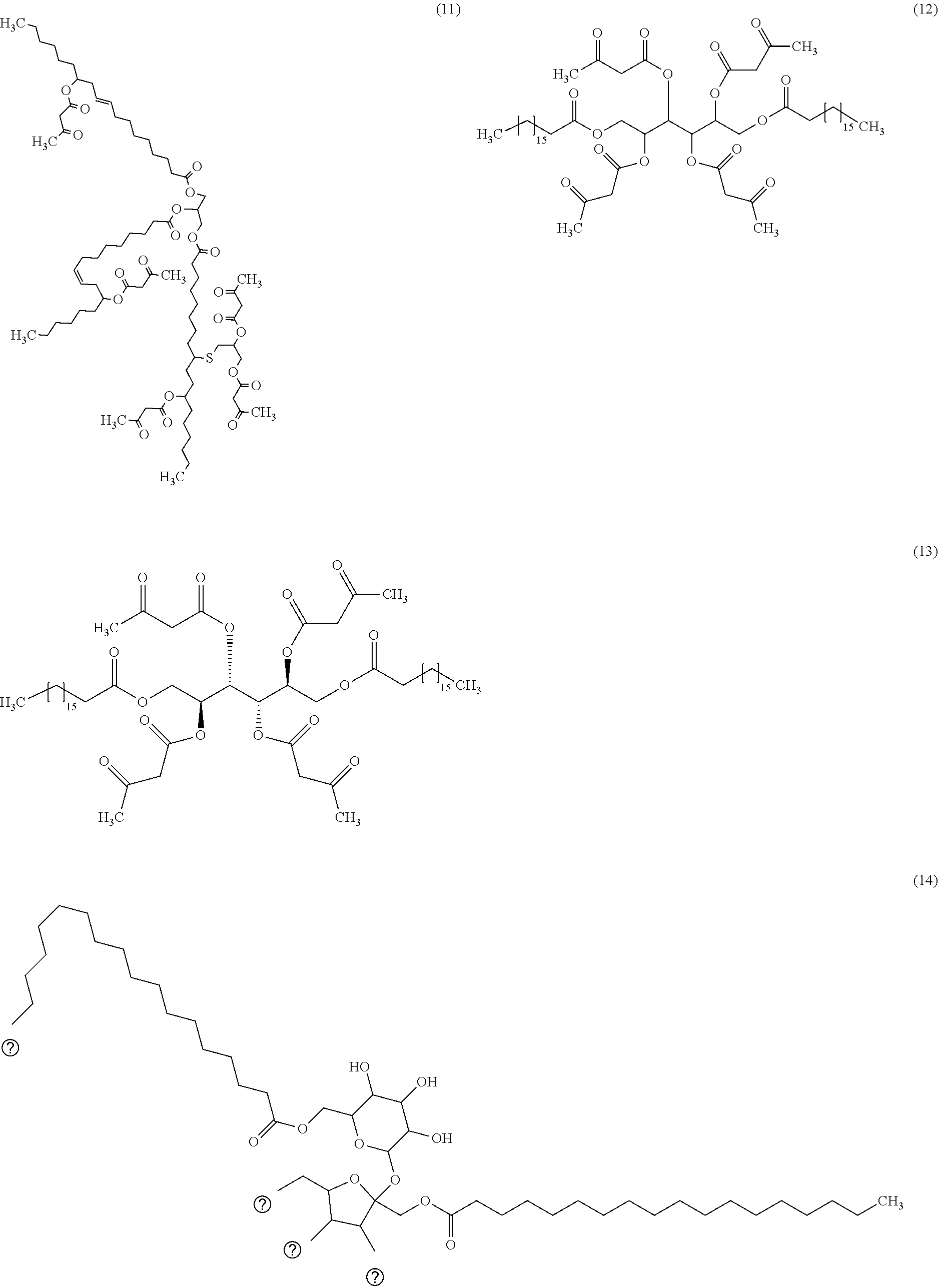 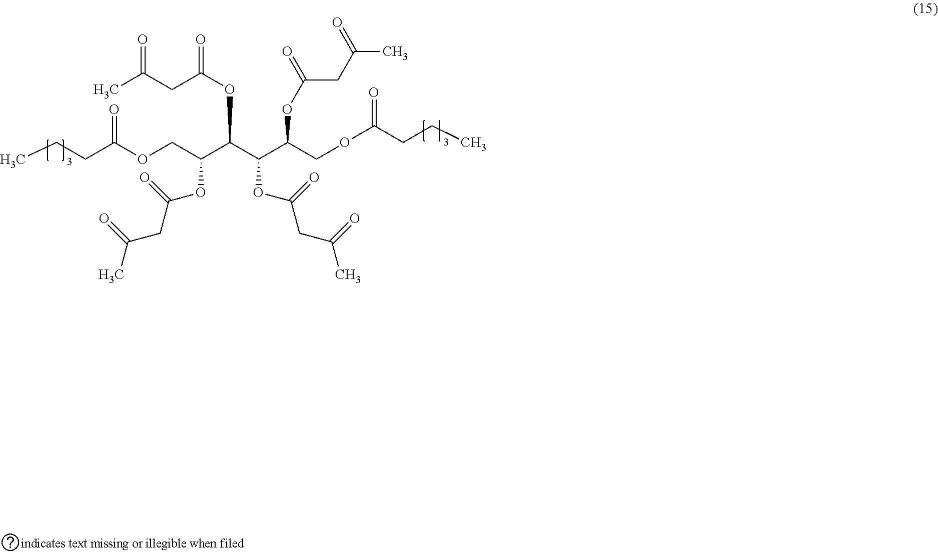
with W being a hydrogen atom or —C(O)—C(Rₐ)(R_{b})—C(O)—R₄, wherein said compound comprises at least two groups —C(O)—C(Rₐ)(R_{b})—C(O)—R₄.

39. The method of claim 25, wherein the ii) one or more crosslinking agents is chosen from (poly)amine, (poly)thiol, (poly)carbonyl, (poly)acrylate and metal alkoxide compounds, or mixtures thereof.

40. The method of claim 25, wherein the ii) one or more crosslinking agents is chosen from:
A) (poly)amine compounds chosen from:
ia) chitosans,
ib) polyether diamines,
ic) polyether triamines,
id) aminoalkoxysilanes,
ie) 4,7,10-trioxa-1,13-tridecanediamine, or
if) polydialkylsiloxanes comprising primary amine groups at the end of the chain or on side chains, or
mixtures thereof;
B) (poly)thiol compounds chosen from:
iia) polydialkylsiloxanes bearing thiol functions,
iib) alkoxysilanes bearing thiol functions,
ALK₁ represents a linear or branched, optionally cyclic, saturated or unsaturated divalent hydrocarbon-based chain comprising from 1 to 100 carbon atoms, optionally interrupted with one or more heteroatoms such as oxygen, sulfur or nitrogen,
n and m are independently chosen from an integer greater than 2, or mixtures thereof;
C) (poly)acrylate compounds of formula (XIV): 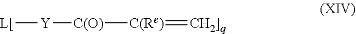
wherein in formula (XIV):
q represents an integer greater than or equal to 2,
L denotes a linear or branched, saturated or unsaturated, or (hetero) cyclic, saturated or unsaturated, multivalent (at least divalent) group; L being optionally interrupted and/or terminated with one or more heteroatoms or groups chosen from O, S, N, Si, C(X), or combinations thereof; and/or L being optionally substituted with one or more groups chosen from: —N(Rₐ) R_{b} and —(X′)ₐ—C(X)—(X″)_{b}—Rₐ; with X, X′ and X″, which may be identical or different, representing an oxygen or sulfur atom, or a group N(R_{b}); a and b being 0 or 1; Rₐ and R_{b} are independently chosen from a hydrogen atom, a (C₁-C₆)alkyl group, or an aryl(C₁-C₄)alkyl group,
Rₑ represents a hydrogen atom or a (C₁-C₄)alkyl group; and
Y represents an oxygen atom or an amino group —N(H)—,
D) metal alkoxides chosen from: zirconium ethoxide (Zr(OC₂H₅)₄), zirconium propoxide (Zr(OCH₂CH₂CH₃)₄), zirconium isopropoxide (Zr(OCH(CH₃)₂)₄), zirconium butoxide Zr(OCH₂CH₂CH₂CH₃)₄, zirconium tert-butoxide (Zr(OC(CH₃)₃)₄), titanium ethoxide (Ti(OC₂H₅)₄), titanium propoxide (Ti(OCH₂CH₂CH₃)₄), titanium isopropoxide (Ti(OCH(CH₃)₂)₄), titanium butoxide (Ti(OCH₂CH₂CH₂CH₃)₄), titanium tert-butoxide (Ti(OC(CH₃)₃)₄), titanium 2-ethylhexyloxide (Ti(OCH₂CH(C₂H₅)(CH₂)₃CH₃)₄), or mixtures thereof;
E) (poly)carbonyl compounds chosen from (poly)carbonyl compounds comprising a saturated or unsaturated, aromatic C₅-C₇ carbocycle; or
mixtures thereof.

41. The method of claim 25, wherein the composition further comprises iii) one or more cosmetic active agents chosen from:
a) dyestuffs,
b) active agents for caring for keratin materials,
c) UV-screening agents, or
mixtures thereof.

42. The method of claim 41, wherein the a) dyestuffs is chosen from pigments, direct dyes or mixtures thereof.

43. The method of claim 25, wherein the composition further comprises iv) one or more fatty substances.

44. A multi-compartment kit comprising:
a first compartment comprising i) one or more compounds of formula (I), optical or geometrical isomers thereof, solvates thereof, or mixtures thereof: 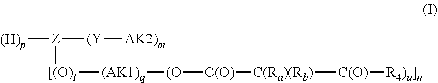
wherein in formula (I):
Z represents a linear or branched, saturated or unsaturated, conjugated or non-conjugated, acyclic or cyclic, aromatic or non-aromatic multivalent C2 to C40, hydrocarbon-based radical, Z possibly being interrupted with one or more groups or heteroatoms chosen from —O—, —N(Rₐ)—, —S—, —C(O)—, (hetero)cycles, or combinations thereof;
AK1 represents a divalent saturated or unsaturated, linear or branched C₁-C₁₂, hydrocarbon-based chain;
AK2 represents a linear or branched, saturated or unsaturated, C₁-C₄₀ monovalent hydrocarbon-based chain, optionally substituted with one or more radicals independently chosen from —OR; —X-AK3-(O—C(O)—C(Rₐ)(R_{b})—C(O)—R₄)ᵥ; or said chain being optionally interrupted with one or more heteroatoms or groups chosen from —O—, —S—, —C(O)—, (hetero)cycle(s), or combinations thereof;
AK3 represents a divalent linear or branched, saturated or unsaturated C₁-C₁₂, hydrocarbon-based chain;
R represents a hydrogen atom, a linear or branched, saturated or unsaturated C₁-C₆, alkyl radical, or a radical —C(O)—C(Rₐ)(R_{b})—C(O)—R₄;
X represents —O—, —S—, —O—C(O)— or —C(O)—O—;
Y represents —O—C(O)— or —C(O)—O—;
R₄ represents a linear or branched, saturated or unsaturated C₁-C₆ monovalent hydrocarbon-based radical;
Rₐ and R_{b} are independently chosen from a hydrogen atom or a (C₁-C₄)alkyl group;
p denotes an integer equal to 0 or 1;
m denotes an integer equal to 0, 1, 2, 3 or 4; wherein when m is greater than 1, then the radicals —Y-AK₂ are identical or different;
n denotes an integer equal to 0, 1, 2, 3 or 4; wherein when n is greater than 1, then the radicals —(O)ₜ-(AK₁)_{q}-(O—C(O)—C(Rₐ)(R_{b})—C(O)—R₄)ᵤ are identical or different;
t denotes an integer equal to 0 or 1;
q denotes an integer equal to 0 or 1;
u denotes an integer equal to 1, 2 or 3;
v denotes an integer equal to 1, 2 or 3;
with the proviso that if q is equal to 0 then t is equal to 0, that (m+n) is greater than or equal to 1, and that the compounds of formula (I) comprises at least two groups —C(O)—C(Rₐ)(R_{b})—C(O)—R₄, and optionally iii) one or more cosmetic active agents chosen from:
a) dyestuffs,
b) active agents for caring for keratin materials,
c) UV-screening agents, or
mixtures thereof;
a second compartment different from the first compartment, the second compartment comprising ii) one or more crosslinking agents chosen from (poly)amine, (poly)thiol, (poly)carbonyl, (poly)acrylate and metal alkoxide compounds, or mixtures thereof and optionally the iii) one or more cosmetic active agents as defined above; and
optionally, a third compartment different from the first compartment and the second compartment, the third compartment comprising iii) one or more cosmetic active agents chosen from:
a) dyestuffs,
b) active agents for caring for keratin materials,
c) UV-screening agents, or
mixtures thereof.
